(19) United States
(12) Patent Application Publication
(10) Pub. No.: US 2022/0387312 A1
(43) Pub. Date: Dec. 8, 2022

(54) GASTRIC RESIDENCE SYSTEMS HAVING ARMS WITH CONTROLLED STIFFNESS FOR IMPROVED GASTRIC RESIDENCE

(71) Applicant: Lyndra Therapeutics, Inc., Watertown, MA (US)

(72) Inventors: Rosemary KANASTY, Cambridge, MA (US); Tyler GRANT, Arlington, MA (US); Juan Jaramillo MONTEZCO, Cambridge, MA (US); David C. DUFOUR, Watertown, MA (US); David ALTREUTER, Wayland, MA (US); Nupura BHISE, Watertown, MA (US); Jung Hoon YANG, Watertown, MA (US)

(21) Appl. No.: 17/774,445

(22) PCT Filed: Nov. 6, 2020
(86) PCT No.: PCT/US2020/059534

§ 371 Date: May 4, 2022

Related U.S. Application Data
(60) Provisional application No. 62/933,210, filed on Nov. 8, 2019.

(51) Int. Cl. A61K 9/00; A61K 47/10; A61K 47/38; A61K 47/12

(52) U.S. Cl. CPC A61K 9/0065; A61K 47/10; A61K 47/38; A61K 47/12

(57) ABSTRACT

Provided are gastric residence dosage forms comprising flexible arms that can help prevent premature passage of the gastric residence system through the pylorus of a patient. In particular, described herein are gastric residence systems comprising one or more arms extending radially, the one or more arms comprising a first segment comprising a first polymer composition and a second segment comprising a second polymer composition, wherein the first segment has a stiffness of greater than a stiffness of the second segment, as measured using a 3-point bending test per ASTM D790. The second segments of the arms of the gastric residence systems help prevent premature passage of the gastric residence system through the pylorus of a patient.

### CROSS-REFERENCE TO RELATED APPLICATIONS

This application claims priority benefit of U.S. Provisional Patent Application No. 62/933,210 filed Nov. 8, 2019. The entire contents of that application are hereby incorporated by reference herein.

### FIELD OF THE INVENTION

This relates to gastric residence systems, and more particularly, to gastric residence dosage forms having flexible arms for improved gastric residence.

### BACKGROUND OF THE INVENTION

Gastric residence systems are delivery systems for agents which remain in the stomach for days to weeks, or even over longer periods, during which time drugs or other agents can elute from the systems for absorption in the gastrointestinal tract. Examples of such systems are described in International Patent Application Nos. WO 2015/191920, WO 2015/191925, WO 2017/070612, WO 2017/100367, and PCT/US2017/034856.

Gastric residence systems are designed to be administered to the stomach of a patient, typically in a capsule which is swallowed or introduced into the stomach by an alternate method of administration (for example, feeding tube or gastric tube). Upon dissolution of the capsule in the stomach, the systems expand or unfold to a size which remains in the stomach and resists passage through the pyloric sphincter over the desired residence period (such as three days, seven days, two weeks, etc.). This requires mechanical stability over the desired residence period. Over the period of residence, the system releases an agent or agents, such as one or more drugs, preferably with minimal burst release, which requires careful selection of the carrier material for the agent in order to provide the desired release profile. While resident in the stomach, the system should not interfere with the normal passage of food or other gastric contents. The system should pass out of the stomach at the end of the desired residence time, and be readily eliminated from the patient. If the system prematurely passes from the stomach into the small intestine, it should not cause intestinal obstruction, and again should be readily eliminated from the patient. These characteristics require careful selection of the materials from which the system is constructed, and the dimensions and arrangement of the system.

### SUMMARY OF THE INVENTION

Provided are gastric residence systems having optimized arm stiffness and methods of preparing gastric residence dosage forms having optimized arm stiffness. In particular, gastric residence dosage forms having optimally stiff arms described herein may help improve the gastric residence of the gastric residence forms. By controlling the stiffness of the arms of a gastric residence system, the gastric residence of the gastric residence system may be better controlled. Specifically, flexible arms can help provide a more consistent gastric residence time and/or a longer gastric residence time. Thus, gastric residence systems including arms having a controlled stiffness provided herein may provide more predictable and/or controllable gastric residence times. Gastric residence systems having predictable and/or controllable gastric residence times can increase the reliability and efficacy of the gastric residence system to help ensure that the therapeutic agent is delivered to the patient as intended.

Conversely, gastric residence systems having relatively stiff arms and a relatively flexible core have been shown to bend into configurations (due to gastric waves/forces) small enough to prematurely pass through the pylorus. When the relatively stiff arms are subjected to compression forces, the compression forces are transferred to the relatively flexible core. Thus, configuring gastric residence systems with relatively stiff arms and relatively flexible cores may contribute to variability in gastric residence.

Accordingly, provided herein are gastric residence systems having controlled stiffness that can resist premature passage through a pylorus. The flexible arms may comprise a relatively stiff, or first, portion at a proximal end and a relatively flexible, or second, portion at a distal end, wherein the arms of a gastric residence system extend radially outwards from a proximal end. When subjected to compression forces, the second segment of the arms absorbs some of the force. This allows the second segment to bend, but the first segment can maintain its shape without bending (depending on the magnitude of the force), allowing the gastric residence system to maintain a configuration too large to prematurely mass through a patient's pylorus. Accordingly, gastric residence systems comprising flexible arms disclosed herein may be more able to provide consistent and accurate residence times, improving the reliability and efficacy of the gastric residence system.

In some embodiments, a gastric residence system is provided, the gastric residence system comprising: one or more arms extending radially, wherein the one or more arms comprises a first segment comprising a first polymer composition and a second segment comprising a second polymer composition, wherein the first segment has a stiffness that is greater than a stiffness of the second segment, as measured using a 3-point bending test per ASTM D790.

In some embodiments of the gastric residence system, the gastric residence system comprises a core.

In some embodiments of the gastric residence system, the gastric residence system comprises a plurality of arms connected to the core and extending radially from the core.

In some embodiments of the gastric residence system, each arm of the plurality of arms comprises a first segment comprising a first polymer composition and a second segment comprising a second polymer composition.

In some embodiments of the gastric residence system, the force required to compress the gastric residence system into a configuration small enough to pass through an opening having a diameter of 20 mm is at least 1.2 times greater than the force required to compress a gastric residence system having only a first polymer composition into a configuration small enough to pass through the opening, as measured using an iris testing mechanism.

In some embodiments of the gastric residence system, the force required to compress the gastric residence system into a configuration small enough to pass through an opening having a diameter of 20 mm is 5-10 N, as measured using an iris testing mechanism.

In some embodiments of the gastric residence system, the first polymer composition comprises one or more of PCL, PLA, PLGA, HPMCAS, and TPU.

In some embodiments of the gastric residence system, the second polymer composition comprises one or more of a polyurethane, a polyether-polyamide copolymer, a thermoplastic elastomer, a thermoplastic polyurethane, polycaprolactone polylactic acid copolymer, a poly(trimethylene carbonate), a polyglycerol sebacate, and a silicone.

In some embodiments of the gastric residence system, the second polymer composition comprises at least a polycaprolactone and a soluble material to form a material that softens upon exposure to an aqueous environment.

In some embodiments of the gastric residence system, the first segment is directly connected to the second segment of the at least one arm.

In some embodiments of the gastric residence system, the first segment is connected to the second segment via a linker.

In some embodiments of the gastric residence system, the first segment comprises 20-50% of a length of the at least one arm, the length being measured from a proximal end of the at least one arm, the proximal end being proximate to the core or a linker connecting the at least one arm to the core, to a distal end of the at least one arm.

In some embodiments of the gastric residence system, the second segment comprises 50-80% of a length of the at least one arm, the length being measured from a proximal end of the at least one arm, the proximal end being proximate to the core or a linker connecting the at least one arm to the core, to a distal end of the at least one arm.

In some embodiments of the gastric residence system, a number of fatigue cycles required to break the gastric residence system is at least 25% greater than a number of fatigue cycles required to break a gastric residence system with stiff arms, as measured using a double funnel test.

In some embodiments of the gastric residence system, a filament circumferentially connecting the distal end of each arm of the plurality of arms.

In some embodiments of the gastric residence system, the force required to compress the gastric residence system into a configuration small enough to pass through an opening having a diameter of 20 mm is at least one and a half times greater than the force required to compress a gastric residence system without a filament into a configuration small enough to pass through the opening, as measured using an iris testing mechanism.

In some embodiments of the gastric residence system, the distal end of each arm of the plurality of arms comprises an enteric polymer composition.

In some embodiments of the gastric residence system, the filament comprises one or more of an elastic polymer, a biosorbable polymer, and a plasticizer.

In some embodiments of the gastric residence system, the enteric polymer composition comprises a biodegradable polymer, an enteric polymer, a plasticizer, and an acid.

In some embodiments of the gastric residence system, the biodegradable polymer comprises polycaprolactone.

In some embodiments of the gastric residence system, the enteric polymer comprises hydroxypropylmethylcellulose acetate succinate.

In some embodiments of the gastric residence system, the plasticizer comprises propylene glycol.

In some embodiments of the gastric residence system, the acid comprises stearic acid.

In some embodiments of the gastric residence system, the pullout force required to separate the filament from the distal end of a first arm of the plurality of arms is greater than 1N when measured after incubating the gastric residence system in an environment of pH 1.6 for 3 days.

In some embodiments of the gastric residence system, the pullout force required to separate the filament from the distal end of the first arm of the plurality of arms is less than 2N when measured after incubating the gastric residence system in an environment of pH 6.5 for 3 days.

In some embodiments of the gastric residence system, the distal end of each arm comprises a notch and the filament is positioned within the notch of each distal end.

In some embodiments of the gastric residence system, the filament is secured by overlapping a first end of the filament and a second end of the filament within a first notch, and the first end and the second end are secured by one of knotting or heat flaring.

In some embodiments of the gastric residence system, the gastric residence system is used to treat a patient.

In some embodiments of the gastric residence system, the patient is a human or a dog.

In some embodiments, a method of manufacturing a gastric residence system is provided, the method comprising: connecting a first material comprising a first polymer composition to a second material comprising a second polymer composition to form a first arm comprising a first segment at a first end of the arm and a second segment at a second end of the first arm; connecting a third material comprising a third polymer composition to a fourth material comprising a fourth polymer composition to form a second arm comprising a third segment at a third end of the arm and a fourth portion at a fourth end of the second arm; connecting the first end of the first arm and the third end of the second arm to a core to form a gastric residence system comprising a core, a first arm, and a second arm, the first arm and the second arm extending radially from the core, wherein the first segment of the first arm comprises a first proximal end and the third segment of the second arm comprises a second proximal end.

In some embodiments of the method, the first material and the third material are the same and the first polymer composition and the third polymer composition are the same.

In some embodiments of the method, the second material and the fourth material are the same and the second polymer composition and the fourth polymer composition are the same.

In some embodiments of the method, the first segment and the third segment have a stiffness of greater a stiffness of the second segment and the fourth portion, as measured using a 3-point bending test per ASTM D790.

In some embodiments of the method, the gastric residence system comprises more than two arms connected to the core and extending radially from the core.

In some embodiments of the method, each arm of more than two arms comprises a first segment comprising a first polymer composition and a second segment comprising a second polymer composition or a third segment comprising a third polymer composition and a fourth portion comprising a fourth polymer composition.

In some embodiments of the method, the force required to compress the gastric residence system into a configuration small enough to pass through an opening having a diameter of 20 mm is at least 1.2 times greater than the force required to compress a gastric residence system having arms comprising only a first polymer composition or a third polymer composition into a configuration small enough to pass through the opening, as measured using an iris testing mechanism.

In some embodiments of the method, the force required to compress the gastric residence system into a configuration small enough to pass through an opening having a diameter of 20 mm is 5-10 N, as measured using an iris testing mechanism.

In some embodiments of the method, the first polymer composition and the third polymer composition comprise one or more of PCL, PLA, PLGA, HPMCAS, and TPU.

In some embodiments of the method, the second polymer composition and the fourth polymer composition comprise one or more of a polyurethane, a polyether-polyamide copolymer, a thermoplastic elastomer, a thermoplastic polyurethane, polycaprolactone polylactic acid copolymer, a poly(trimethylene carbonate), a polyglycerol sebacate, and a silicone.

In some embodiments of the method, the second polymer composition and the fourth polymer composition comprise at least a polycaprolactone and a soluble material to form a material that softens upon exposure to an aqueous environment.

In some embodiments of the method, the first segment is directly connected to the second segment of the first arm and the third segment is directly connected to the fourth portion of the second arm.

In some embodiments of the method, the first segment is connected to the second segment via a linker and the third segment is connected to the fourth portion via a linker.

In some embodiments of the method, the first segment comprises 20-50% of a length of the first arm, the length being measured from a proximal end of the first arm, the proximal end being proximate to the core, to a distal end of the first arm.

In some embodiments of the method, the third segment comprises 20-50% of a length of the second arm, the length being measured from a proximal end of the second arm, the proximal end being proximate to the core, to a distal end of the second arm.

In some embodiments of the method, the second segment comprises 50-80% of a length of the first arm, the length being measured from a proximal end of the first arm, the proximal end being proximate to the core, to a distal end of the first arm.

In some embodiments of the method, the fourth portion comprises 50-80% of a length of the second arm, the length being measured from a proximal end of the second arm, the proximal end being proximate to the core, to a distal end of the second arm.

In some embodiments of the method, a number of fatigue cycles required to break the gastric residence system is at least 25% greater than a number of fatigue cycles required to break a gastric residence system with arms comprising only a first segment or a third segment, as measured using a double funnel test.

In some embodiments of the method, the method comprises wrapping a filament circumferentially connecting the distal end of the first arm and the second arm.

In some embodiments of the method, the method comprises wrapping a filament circumferentially connecting the distal end of each of the more than two arms.

In some embodiments of the method, the force required to compress the gastric residence system into a configuration small enough to pass through an opening having a diameter of 20 mm is at least 1.2 times greater than the force required to compress a gastric residence system comprising arms having only a first polymer composition or a third polymer composition and without a filament into a configuration small enough to pass through the opening, as measured using an iris testing mechanism.

In some embodiments of the method, the distal end of each arm of the first arm and the second arm comprises an enteric polymer composition.

In some embodiments of the method, the distal end of each arm of the two or more arms comprises an enteric polymer composition.

In some embodiments of the method, the filament comprises one or more of an elastic polymer, a biosorbable polymer, and a plasticizer.

In some embodiments of the method, the enteric polymer composition comprises a biodegradable polymer, an enteric polymer, a plasticizer, and an acid.

In some embodiments of the method, the biodegradable polymer comprises polycaprolactone.

In some embodiments of the method, the enteric polymer comprises hydroxypropylmethylcellulose acetate succinate.

In some embodiments of the method, the plasticizer comprises propylene glycol.

In some embodiments of the method, the acid comprises stearic acid.

In some embodiments of the method, the pullout force required to separate the filament from the distal end of the first arm or the second arm is greater than 1N when measured after incubating the gastric residence system in an environment of pH 1.6 for 3 days.

In some embodiments of the method, the pullout force required to separate the filament from the distal end of the first arm or the second arm is less than 2N when measured after incubating the gastric residence system in an environment of pH 6.5 for 3 days.

In some embodiments of the method, the distal end of the first arm and the distal end of the second arm comprises a notch, and the filament is positioned within the notch of each distal end.

In some embodiments of the method, the filament is secured by overlapping a first end of the filament and a second end of the filament within a first notch, and the first end and the second end are secured by one of knotting or heat flaring.

In some embodiments of the method, the gastric residence system is used to treat a patient.

In some embodiments of the method, the patient is a human or a dog.

### BRIEF DESCRIPTION OF THE DRAWINGS

The invention will now be described, by way of example only, with reference to the accompanying drawings, in which:

FIGS. 1A, 1B, and 1C show various gastric residence system configurations, according to some embodiments;

FIG. 2 shows a gastric residence system in an open configuration, according to some embodiments;

FIGS. 3A, 3B, and 3C show various methods by which a gastric residence system may pass through a pylorus prior to dissolving, according to some embodiments;

FIGS. 4A and 4B show how the bending profile of a gastric residence system can be altered by modifying the stiffness of the arms of a gastric residence system, according to some embodiments;

FIGS. 5A, 5B, and 5C show various gastric residence system bending profiles, according to some embodiments;

FIG. 6 shows a method of measuring stiffness of a gastric residence system using a 3-point being test, according to some embodiments;

FIG. 7 shows an iris mechanism measuring radial force of a gastric residence system, according to some embodiments;

FIG. 8 shows a method of measuring the durability of a gastric residence system using cyclic loading in a double funnel, according to some embodiments;

FIG. 9 shows a method of measuring the durability of a gastric residence system using cyclic loading of a planar circumferential bend, according to some embodiments;

FIG. 10 shows material stiffness data of different gastric residence systems, according to some embodiments;

FIG. 11 shows the radial force of various iris diameters for a gastric residence system having relatively stiff arms and a gastric residence system having relatively flexible arms (i.e., a first segment and a second segment), according to some embodiments;

FIG. 12 shows failure mode analysis data of gastric residence systems having relatively stiff arms and gastric residence systems having relatively flexible arms, according to some embodiments;

FIG. 13 shows the number of cycles to failure for gastric residence systems having relatively stiff arms and gastric residence systems having relatively flexible arms, according to some embodiments;

FIG. 14 shows the release profile of dapagliflozin for gastric residence systems having a PCL coating, according to some embodiments;

FIG. 15 shows the amount of dapagliflozin per day for uncoated gastric residence systems and coated gastric residence systems, according to some embodiments;

FIG. 16 shows the linearity of dapagliflozin release per day for coated and uncoated gastric residence systems, according to some embodiments;

FIG. 17 shows the release profile of ivermectin from gastric residence systems having elastic TPU-based matrices, according to some embodiments;

FIG. 18 shows the ivermectin release profile for gastric residence systems made with TPU of different durometers, according to some embodiments; and

FIG. 19 shows the ivermectin release profile for arms/gastric residence systems comprising TPU or PCL polymers, according to some embodiments.

### DETAILED DESCRIPTION OF THE INVENTION

Described herein are gastric residence systems and methods of preparing gastric residence systems having arms of a controlled stiffness to better control gastric residence. As described above, gastric residence systems are designed to reside in the gastrointestinal tract for a predetermined amount of time. After a period of time (e.g., the predetermined residence time), the gastric residence system breaks down into several pieces small enough to pass through the pylorus. However, if the stomach can compress a gastric residence system to a size small enough, the entire gastric residence system may pass through the pylorus prematurely. For a given core, a gastric residence system with arms that are relatively stiff throughout their length can be compressed to pyloric size more easily than a gastric residence system with arms that have a relatively flexible segment (e.g., at a distal end of the arm), due to the difference in effective lever arm length.

Additionally, gastric residence systems comprising arms of controlled stiffness have been shown to increase the durability of the gastric residence system. In particular, having arms of controlled stiffness can help minimize failure of a gastric residence system due to repeated flexing and/or compression in the stomach, for example. Both the incidence of core tearing and weld breaks are decreased in gastric residence systems having arms of controlled stiffness.

Accordingly, gastric residence systems provided herein comprise arms having a controlled stiffness that can help prevent a gastric residence system from passing through the pylorus before the predetermined residence time has expired and to improve the durability of the gastric residence system.

Gastric residence systems are typically administered in a folded, closed, or collapsed configuration. When the gastric residence system enters the patient's stomach, it unfolds to assume an open configuration. The physical opening or unfolding of the gastric residence system results in a dosage form with effective size (i.e., a gastric residence system in an open configuration) that is too large to pass through the patient's pyloric valve (i.e., opening between the stomach and the large intestine). The deployed, or expanded, gastric residence system can stay in the patient's stomach for a predetermined period of time (e.g., 24 hours, 48 hours, 7 days, 10 days, etc.).

However, one challenge in particular with gastric residence systems is ensuring a consistent and accurate residence time. A gastric residence system that passes through the pylorus too early fails to administer the intended amount of therapeutic agent, comprising the efficacy and reliability of the gastric residence system.

Accordingly, gastric residence systems provided herein are designed for more consistent and accurate residence times in a patient's stomach. In particular, gastric residence systems comprising arms of a controlled stiffness provided herein are more likely to resist premature passage through the pylorus. Thus, gastric residence systems provided herein are more likely to deliver consistent and accurate residence times, improving the efficacy and reliability of the gastric residence system.

### Definitions

As used herein, “gastric residence system” is a dosage form comprising a therapeutic agent and is configured to be administered to a patient in a folded configuration. A “gastric residence dosage form” comprises a folded gastric residence system and is configured to hold the gastric residence system in a folded configuration until deployment. For example, a gastric residence dosage form may comprise a capsule and/or a capsule coating according to those described in U.S. Appln. No. 62/821,352 titled “Capsules and Capsule Coatings for Gastric Residence Dosage Forms” and/or U.S. Appln. No. 62/821,361 titled “Coatings for Gastric Residence Forms.”

A “carrier polymer” is a polymer suitable for blending with an agent, such as a drug, for use in the invention.

An “agent” is any substance intended for therapeutic, diagnostic, or nutritional use in a patient, individual, or subject. Agents include, but are not limited to, drugs, nutrients, vitamins, and minerals.

A “dispersant” is defined as a substance which aids in the minimization of particle size of agent and the dispersal of agent particles in the carrier polymer matrix. That is, the dispersant helps minimize or prevent aggregation or flocculation of particles during fabrication of the systems. Thus, the dispersant has anti-aggregant activity and anti-flocculant activity, and helps maintain an even distribution of agent particles in the carrier polymer matrix.

An “excipient” is any substance added to a formulation of an agent that is not the agent itself. Excipients include, but are not limited to, binders, coatings, diluents, disintegrants, emulsifiers, flavorings, glidants, lubricants, and preservatives. The specific category of dispersant falls within the more general category of excipient.

An “elastic polymer” or “elastomer” (also referred to as a “tensile polymer”) is a polymer that is capable of being deformed by an applied force from its original shape for a period of time, and which then substantially returns to its original shape once the applied force is removed.

A “coupling polymer” is a polymer suitable for coupling any other polymers together, such as coupling a first carrier polymer-agent component to a second carrier polymer-agent component. Coupling polymers typically form the linker regions between other components.

A “time-dependent polymer” or “time-dependent coupling polymer” is a polymer that degrades in a time-dependent manner when a gastric residence system is deployed in the stomach. A time-dependent polymer is typically not affected by the normal pH variations in the stomach.

“Approximately constant plasma level” refers to a plasma level that remains within a factor of two of the average plasma level (that is, between 50% and 200% of the average plasma level) measured over the period that the gastric residence system is resident in the stomach.

“Substantially constant plasma level” refers to a plasma level that remains within plus-or-minus 25% of the average plasma level measured over the period that the gastric residence system is resident in the stomach.

A “hydrophilic therapeutic agent,” “hydrophilic agent,” or “hydrophilic drug” is an agent which readily dissolves in water. A hydrophilic agent is defined as an agent which has a solubility in water of 1 mg/ml or greater. Alternatively, a hydrophilic agent can be defined as an agent which has a log Poct (log partition coefficient Poct, where Poct=(concentration in 1-octanol)/(concentration in H2O)) in a 1-octanol/water system of less than 0.5. The pH at which solubility or log Poct is measured is 1.6, approximating the gastric environment.

A “hydrophobic therapeutic agent,” “hydrophobic agent,” or “hydrophobic drug” is an agent which does not readily dissolve in water. A hydrophobic agent is defined as an agent which has a solubility in water of less than 1 mg/ml. Alternatively, a hydrophobic agent can be defined as an agent which has a log Poct (log partition coefficient) in a 1-octanol/water system of greater than 1. Alternatively, a hydrophobic therapeutic agent can be defined as an agent which has a higher solubility in ethanol than in water. Alternatively, a hydrophobic therapeutic agent can be defined as an agent which has a higher solubility in 40% ethanol/60% simulated gastric fluid than in 100% simulated gastric fluid.

“Biocompatible,” when used to describe a material or system, indicates that the material or system does not provoke an adverse reaction, or causes only minimal, tolerable adverse reactions, when in contact with an organism, such as a human. In the context of the gastric residence systems, biocompatibility is assessed in the environment of the gastrointestinal tract.

A “patient,” “individual,” or “subject” refers to a mammal, preferably a human or a domestic animal such as a dog or cat. In a most preferred embodiment, a patient, individual, or subject is a human.

The “diameter” of a particle as used herein refers to the longest dimension of a particle.

“Treating” a disease or disorder with the systems and methods disclosed herein is defined as administering one or more of the systems disclosed herein to a patient in need thereof, with or without additional agents, in order to reduce or eliminate either the disease or disorder, or one or more symptoms of the disease or disorder, or to retard the progression of the disease or disorder or of one or more symptoms of the disease or disorder, or to reduce the severity of the disease or disorder or of one or more symptoms of the disease or disorder. “Suppression” of a disease or disorder with the systems and methods disclosed herein is defined as administering one or more of the systems disclosed herein to a patient in need thereof, with or without additional agents, in order to inhibit the clinical manifestation of the disease or disorder, or to inhibit the manifestation of adverse symptoms of the disease or disorder. The distinction between treatment and suppression is that treatment occurs after adverse symptoms of the disease or disorder are manifest in a patient, while suppression occurs before adverse symptoms of the disease or disorder are manifest in a patient. Suppression may be partial, substantially total, or total. Because some diseases or disorders are inherited, genetic screening can be used to identify patients at risk of the disease or disorder. The systems and methods of the invention can then be used to treat asymptomatic patients at risk of developing the clinical symptoms of the disease or disorder, in order to suppress the appearance of any adverse symptoms.

“Therapeutic use” of the systems disclosed herein is defined as using one or more of the systems disclosed herein to treat a disease or disorder, as defined above. A “therapeutically effective amount” of a therapeutic agent, such as a drug, is an amount of the agent, which, when administered to a patient, is sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder. A therapeutically effective amount can be administered to a patient as a single dose, or can be divided and administered as multiple doses.

“Prophylactic use” of the systems disclosed herein is defined as using one or more of the systems disclosed herein to suppress a disease or disorder, as defined above. A “prophylactically effective amount” of an agent is an amount of the agent, which, when administered to a patient, is sufficient to suppress the clinical manifestation of a disease or disorder, or to suppress the manifestation of adverse symptoms of a disease or disorder. A prophylactically effective amount can be administered to a patient as a single dose, or can be divided and administered as multiple doses.

A “flexural modulus” of a material is an intrinsic property of a material computed as the ratio of stress to strain in flexural deformation of the material as measured by a 3-point bending test. Although the linkers are described herein as being components of the gastric residence system, the flexural modulus of the material of the polymeric material may be measured in isolation. For example, the polymeric linker in the gastric residence system may be too short to measure the flexural modulus, but a longer sample of the same material may be used to accurately determine the flexural modulus. The longer sample used to measure the flexural modulus should have the same cross-sectional dimensions (shape and size) as the polymeric linker used in the gastric residence system. The flexural modulus is measured using a 3-point bending test in accordance with the ASTM standard 3-point bending test (ASTM D790) using a 10 mm distance between supports and further modified to accommodate materials with non-rectangular cross-sections. The longest line of symmetry for the cross section of the polymeric linker should be positioned vertically, and the flexural modulus should be measured by applying force downward. If the longest line of symmetry for the cross section of the polymeric linker is perpendicular to a single flat edge, the single flat edge should be positioned upward. If the cross-section of the polymeric linker is triangular, the apex of the triangle should be faced downward. As force is applied downward, force and displacement are measured, and the slope at the linear region is obtained to calculate the flexural modulus.

As used herein, the singular forms “a”, “an”, and “the” include plural references unless indicated otherwise or the context clearly dictates otherwise.

When numerical values are expressed herein using the term “about” or the term “approximately,” it is understood that both the value specified, as well as values reasonably close to the value specified, are included. For example, the description “about 50° C.” or “approximately 50° C.” includes both the disclosure of 50° C. itself, as well as values close to 50° C. Thus, the phrases “about X” or “approximately X” include a description of the value X itself. If a range is indicated, such as “approximately 50° C. to 60° C.” or “about 50° C. to 60° C.,” it is understood that both the values specified by the endpoints are included, and that values close to each endpoint or both endpoints are included for each endpoint or both endpoints; that is, “approximately 50° C. to 60° C.” (or “about 50° C. to 60° C.”) is equivalent to reciting both “50° C. to 60° C.” and “approximately 50° C. to approximately 60° C.” (or “about 50° C. to 60° C.”).

With respect to numerical ranges disclosed in the present description, any disclosed upper limit for a component may be combined with any disclosed lower limit for that component to provide a range (provided that the upper limit is greater than the lower limit with which it is to be combined). Each of these combinations of disclosed upper and lower limits are explicitly envisaged herein. For example, if ranges for the amount of a particular component are given as 10% to 30%, 10% to 12%, and 15% to 20%, the ranges 10% to 20% and 15% to 30% are also envisaged, whereas the combination of a 15% lower limit and a 12% upper limit is not possible and hence is not envisaged.

Unless otherwise specified, percentages of ingredients in compositions are expressed as weight percent, or weight/weight percent. It is understood that reference to relative weight percentages in a composition assumes that the combined total weight percentages of all components in the composition add up to 100. It is further understood that relative weight percentages of one or more components may be adjusted upwards or downwards such that the weight percent of the components in the composition combine to a total of 100, provided that the weight percent of any particular component does not fall outside the limits of the range specified for that component.

Partitioning behavior of an agent can be measured between a polycaprolactone phase (PCL phase) and a simulated gastric fluid phase (SGF phase), to give the partition coefficient PPCL-SGF between the two phases for the agent. Log PPCL-SGF can also be calculated. A 5:1 mixture of polycaprolactone diol (MW 530):ethyl acetate can be used as the PCL phase, and fasted-state simulated gastric fluid (FaSSGF) can be used as the SGF phase, such that PPCL-SGF=(concentration in polycaprolactone diol)/(concentration in FaSSGF)).

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition or system is described as “consisting essentially of” the listed elements, the composition or system contains the elements expressly listed, and may contain other elements which do not materially affect the condition being treated (for compositions for treating conditions), or the properties of the described system (for compositions comprising a system). However, the composition or system either does not contain any other elements which do materially affect the condition being treated other than those elements expressly listed (for compositions for treating systems) or does not contain any other elements which do materially affect the properties of the system (for compositions comprising a system); or, if the composition or system does contain extra elements other than those listed which may materially affect the condition being treated or the properties of the system, the composition or system does not contain a sufficient concentration or amount of those extra elements to materially affect the condition being treated or the properties of the system. When a method is described as “consisting essentially of” the listed steps, the method contains the steps listed, and may contain other steps that do not materially affect the condition being treated by the method or the properties of the system produced by the method, but the method does not contain any other steps which materially affect the condition being treated or the system produced other than those steps expressly listed.

This disclosure provides several embodiments. It is contemplated that any features from any embodiment can be combined with any features from any other embodiment where possible. In this fashion, hybrid configurations of the disclosed features are within the scope of the present invention.

In addition to the embodiments and methods disclosed here, additional embodiments of gastric residence systems, and methods of making and using such systems, are disclosed in International Patent Application Nos. WO 2015/191920, WO 2015/191925, WO 2017/070612, WO 2017/100367, and PCT/US2017/034856, which are incorporated by reference herein in their entirety.

### Gastric Residence Systems

Provided herein are arms of gastric residence systems and segments for use in gastric residence systems, in which the arms and segments of the arms have controlled stiffness to help prevent early passage of the gastric residence system through the pylorus. Following is a description of an overall gastric residence system configuration and a detailed description of each of the three main components of a gastric residence system: an elastomer (i.e., central elastomer or core), arms (i.e., elongate members, carrier polymers, or carrier polymer-agent components), and coupling polymers (i.e., linker, linker region, or linker component). More specifically, described herein is: Overall system configuration; system dimensions; Residence time; Evaluation of release characteristics; Gastric Delivery Pharmacokinetics for Gastric Residence Systems; Dissolution Profile, Bioavailability, and Pharmacokinetics for Gastric Residence System; Elastomers; Carrier polymers for segments and arms (carrier polymer-agent component); Controlling Stiffness for Arms of a Gastric Residence System; Carrier polymer-agent/agent salt combinations with excipients and other additives; Agents for use in gastric residence systems; High agent loading of arms and segments; Dispersants for modulation of agent release and stability of polymer blend; Stabilizers for use in gastric residence systems; Coupling polymers; Gastric Residence Arms comprising a Filament; and System polymeric composition.

In some embodiments, a gastric residence system may comprise arms that have both a first segment and a second segment. For example, the first segment may be located at a proximal end of an arm (i.e., proximate to the core of the gastric residence system) and the second segment may be located at a distal end of an arm. In some embodiments, the first segment may have a stiffness that is greater than a stiffness of a second segment. In some embodiments, an entire arm may comprise a single material of different durometers. For example, the arm material at the proximal end may comprise a higher durometer than the arm material at the distal end of the arm. In some embodiments, the stiff portion of an arm may comprise a first material, and the flexible portion of the arm may comprise a second material, wherein the first material has a higher durometer than the second material. In some embodiments, an arm of a gastric residence dosage form may comprise a single material having a constant stiffness throughout the length of the arm. In some embodiments, the thickness of an arm, or the cross-sectional area of the arm, may be less towards a distal end of the arm as compared to the proximal end of the arm.

Gastric residence dosage forms can be designed to be administered to the stomach of a patient by swallowing, by feeding tube, by gastric tube, etc. Once a gastric residence dosage form is in place in the stomach, it can remain in the stomach for a desired residence time (e.g., three days, seven days, two weeks, etc.). A gastric residence dosage form that is properly in place in a stomach will resist passage through the pyloric valve, which separates the stomach from the small intestine. Gastric residence dosage forms can release a therapeutic agent (i.e., API or drug) over the period of residence with controlled release. While residing in the stomach, the dosage form may not interfere with the normal passage of food or other gastric contents. Once the desired residence time has expired, the dosage form passes out of the stomach (i.e., through the pyloric valve) and is readily eliminated from the patient.

To administer a gastric residence system to a patient, the gastric residence system can be folded into a form small enough to be swallowed or otherwise administered. In some embodiments, the folded gastric residence system is retained in a capsule or other container which can be swallowed by the patient. In some cases, the gastric residence system may be delivered to a patient via gastrostomy tube, feeding tube, gastric tube, or other route of administration to the stomach. Specific examples of gastric residence systems may be found in PCT/US2018/051816, WO 2015/191920, WO 2017/070612, WO 2017/100367, WO 2018/064630, WO 2017/205844, WO 2018/227147, each of which is incorporated herein in its entirety.

Once the gastric residence system reaches the stomach of a patient, it may assume an open configuration. The dimensions of an open gastric residence system are, when left unaltered, suitable to prevent passage of the device through the pyloric valve for the period of time during which the device is intended to reside in the stomach. In some embodiments, the folded gastric residence system can also be secured by a dissolvable retaining band or sleeve that can prevent premature deployment of the gastric residence system in case of a failure of the capsule.

While in the stomach, the gastric residence system is compatible with digestion and other normal functioning of the stomach or gastrointestinal tract. The gastric residence system does not interfere with or impede the passage of chyme (partially digested food) or other gastric contents which exit the stomach through the pyloric valve into the duodenum.

Once released from the capsule into the stomach, the therapeutic agent of the gastric residence system begins to take effect. In some embodiments, the gastric residence system comprises a plurality of carrier polymer-agent components. The carrier polymer-agent components may comprise a carrier polymer, a pore former, and a therapeutic agent (or a salt thereof). The plurality of carrier polymer-agent components are linked together by one or more coupling polymer components. The therapeutic agent may be eluted from the carrier polymer-agent components into the gastric fluid of the patient over the desired residence time of the system. Release of the therapeutic agent is controlled by appropriate formulation of the carrier polymer-agent components, including by the use of the dispersant in formulation of the carrier polymer-agent components, and by milling of the therapeutic agent to particles of desired size prior to blending the agent with the carrier polymer and excipient/pore former.

Additionally, coatings can be applied to outer surfaces of the gastric residence system. The coatings can include additional therapeutic agents or agents that can affect the release of therapeutic agents or the residence duration of the gastric residence system.

Once the desired residence time has expired, the gastric residence system passes out of the stomach. To do so, various components of the gastric delivery system are designed to weaken and degrade. The specific dimensions of the system are also taken into consideration. In its intact, open configuration, the gastric residence system is designed to resist passage through the pyloric valve. However, coupling polymer components of the gastric residence system are chosen such that they gradually degrade over the specified residence period in the stomach. When the coupling polymer components are sufficiently weakened by degradation, the gastric residence system loses critical resilience to compression or size reduction and can break apart into smaller pieces. The reduced-size dosage form and any smaller pieces are designed to pass through the pyloric valve. The system then passes through the intestines and is eliminated from the patient. In some embodiments, a gastric residence system may be designed to weaken at specific locations such that the gastric residence system can pass through a pyloric valve intact once the residence time expires without degrading into numerous smaller pieces.

### Overall System Configuration

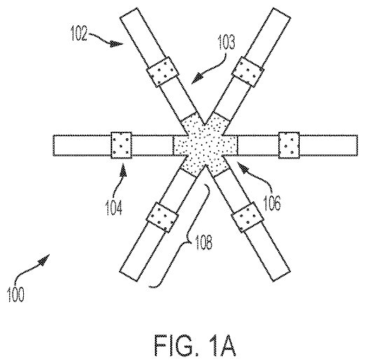

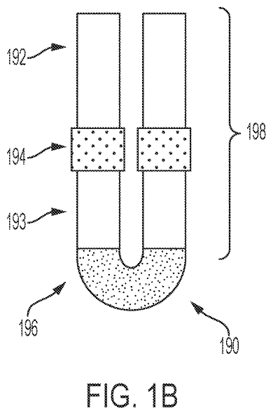

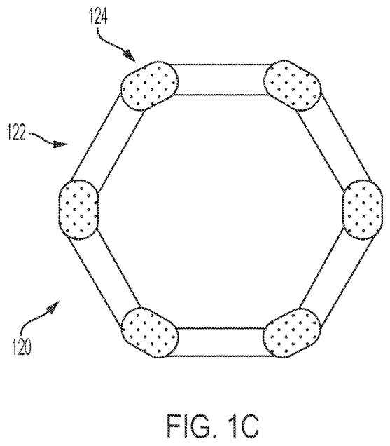

Gastric residence systems can be prepared in different configurations. The “stellate” configuration of a gastric residence system is also known as a “star” (or “asterisk”) configuration. An example of a stellate system 100 is shown schematically in FIG. 1A. Multiple arms (only one such arm, 108, is labeled for clarity), are affixed to disk-shaped central elastomer 106. The arms or arms depicted in FIG. 1A are comprised of segments 102 and 103, joined by a coupling polymer or linker region 104 (again, the components are only labeled in one arm for clarity) which serves as a linker region. This configuration permits the system to be folded or compacted at the central elastomer. FIG. 1B shows a folded configuration 190 of the gastric residence system of FIG. 1A (for clarity, only two arms are illustrated in FIG. 1B). Segments 192 and 193, linker region 194, elastomer 196, and arm 198 of FIG. 1B correspond to segments 102 and 103, linker region 104, elastomer 106, and arm 108 of FIG. 1A, respectively. When folded, the overall length of the system is reduced by approximately a factor of two, and the system can be conveniently placed in a container such as a capsule or other container suitable for oral administration. When the capsule reaches the stomach, the capsule dissolves, releasing the gastric residence system. The gastric residence system then unfolds into its uncompacted state, which is retained in the stomach for the desired residence period.

While the linker regions 104 are shown as slightly larger in diameter than the segments 102 and 103 in FIG. 1A, they can be the same diameter as the segments, so that the entire arm 102-104-103 has a smooth outer surface.

In some embodiments, the stellate system may have an arm composed of only one segment, which is attached to the central elastomer by a linker region. This corresponds to FIG. 1A with the segments 103 omitted. The single-segment arms comprising segments 102 are then directly attached to central elastomer 106 via the linkers 104. The linkers can comprise a coupling polymer or a disintegrating matrix.

A stellate system can be described as a gastric residence system for administration to the stomach of a patient, comprising an elastomer component, and a plurality of at least three carrier polymer-agent components comprising a carrier polymer and an agent or a salt thereof, attached to the elastomer component, wherein each of the plurality of carrier polymer-agent components is an arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end; wherein each arm independently comprises one or more segments, each segment comprising a proximal end, a distal end, and an outer surface therebetween; and wherein, when two or more segments are present in an arm, each segment is attached to an adjacent segment via a linker region. The linker region can be a coupling polymer or a disintegrating matrix. The arms can be attached to the central elastomer via a coupling polymer or a disintegrating matrix, and can have intervening portions of interfacing polymers. For the plurality of at least three arms, or for a plurality of arms, a preferred number of arms is six, but three, four, five, seven, eight, nine, or ten arms can be used. The arms should be equally spaced around the central elastomer; if there are N arms, there will be an angle of about 360/N degrees between neighboring arms.

FIG. 1C shows another possible overall configuration 120 for a gastric residence system, which is a ring configuration. Segments 122 are joined by coupling polymer or linker region 124 (only one segment and one coupling linkage are labeled for clarity). The coupling polymer/linker region in this design must also function as an elastomer, to enable the ring to be twisted into a compacted state for placement in a container, such as a capsule.

In one embodiment of the stellate configuration, the segments 102 and 103 comprise a carrier polymer blended with an agent or drug. In one embodiment of the ring configuration, the segments 122 comprise a carrier polymer blended with an agent or drug.

The coupling polymers of the gastric residence system, which serve as linker regions, are designed to break down gradually in a controlled manner during the residence period of the system in the stomach. If the gastric residence system passes prematurely into the small intestine in an intact form, the system is designed to break down much more rapidly to avoid intestinal obstruction. This is readily accomplished by using enteric polymers as coupling polymers. Enteric polymers are relatively resistant to the acidic pH levels encountered in the stomach, but dissolve rapidly at the higher pH levels found in the duodenum. Use of enteric coupling polymers as safety elements protects against undesired passage of the intact gastric residence system into the small intestine. The use of enteric coupling polymers also provides a manner of removing the gastric residence system prior to its designed residence time; should the system need to be removed, the patient can drink a mildly alkaline solution, such as a sodium bicarbonate solution, or take an antacid preparation such as hydrated magnesium hydroxide (milk of magnesia) or calcium carbonate, which will raise the pH level in the stomach and cause rapid degradation of the enteric coupling polymers. The gastric residence system will then break apart and be eliminated from the patient. In the system shown in FIG. 1A, at least the coupling polymer used for the couplings 104 are made from such enteric polymers.

In additional embodiments, a time-dependent coupling polymer or linker can be used. Such a time-dependent coupling polymer or linker degrades in a predictable, time-dependent manner. In some embodiments, the degradation of the time-dependent coupling polymer or linker may not be affected by the varying pH of the gastrointestinal system.

In additional embodiments, different types of linkers can be used in the gastric residence systems. That is, both enteric linkers (or enteric coupling polymers) and time-dependent linkers (or time-dependent coupling polymers) can be used. In some embodiments, a single multi-segment arm of a stellate system can use both an enteric linker at some linker regions between segments, and a time-dependent linker at other linker regions between segments.

Linker regions are typically about 100 microns to about 1 millimeter in width, such as about 200 um to about 1000 um, about 300 um to about 1000 um, about 400 um to about 1000 um, about 500 um to about 1000 um, about 600 um to about 1000 um, about 700 um to about 1000 um, about 800 um to about 1000 um, or about 900 um to about 1000 um; or about 100 um to about 900 um about 100 um to about 800 um, about 100 um to about 700 um, about 100 um to about 600 um, about 100 um to about 500 um, about 100 um to about 400 um, about 100 um to about 300 um, or about 100 um to about 200 um. Linker regions can be about 100 um, about 200 um, about 300 um, about 400 um, about 500 um, about 600 um, about 700 um, about 800 um, about 900 um, or about 1000 um in width, where each value can be plus or minus 50 um (±50 um).

The central elastomeric polymer of a stellate system is typically not an enteric polymer; however, the central elastomeric polymer can also be made from such an enteric polymer where desirable and practical.

The central elastomer should have a specific durometer and compression set. The durometer is important because it determines the folding force of the dosage form and whether it will remain in the stomach; a preferred range is from about 60 to about 90A. The compression set should be as low as possible to avoid having permanent deformation of the gastric residence system when stored in the capsule in its compacted configuration. A preferred range is about 10% to about 20% range. Materials that fit these requirements are the QP1 range of liquid silicone rubbers from Dow Corning. In any embodiment with a central elastomer, the QP1-270 (70A durometer) liquid silicone rubber can be used.

Segments and arms of the gastric residence systems can have cross-sections in the shape of a circle (in which case the segments are cylindrical), a polygon (such as segments with a triangular cross-section, rectangular cross-section, or square cross-section), or a pie-shaped cross-section (in which case the segments are cylindrical sections). Segments with polygon-shaped or pie-shaped cross-sections, and ends of cylindrically-shaped sections which will come into contact with gastric tissue, can have their sharp edges rounded off to provide rounded corners and edges, for enhanced safety in vivo. That is, instead of having a sharp transition between intersecting edges or planes, an arc is used to transition from one edge or plane to another edge or plane. Thus, a “triangular cross-section” includes cross-sections with an approximately triangular shape, such as a triangle with rounded corners. An arm with a triangular cross-section includes an arm where the edges are rounded, and the corners at the end of the arm are rounded. Rounded corners and edges are also referred to as fillet corners, filleted corners, fillet edges, or filleted edges.

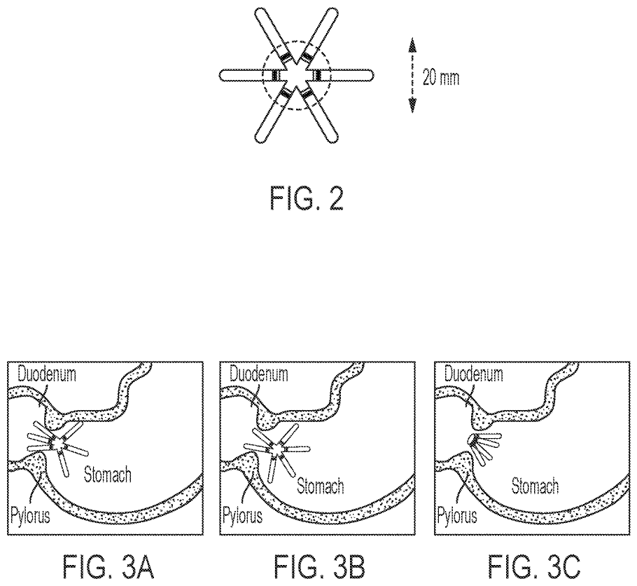

However, it has been determined that some gastric residence systems having relatively stiff arms can bend into configurations that allow for premature passage through the pylorus of a patient. Gastric residence systems that prematurely pass through the pylorus fail to deliver the therapeutic agent of the gastric residence system to the patient as intended. Further, premature passage can cause inconsistency, causes unreliability, and compromises the efficacy of the gastric residence system. FIGS. 2 and 3A-3C, described below, illustrate the issues posed by gastric residence systems having relatively stiff arms.

FIG. 2 shows gastric residence system 200 having relatively stiff arms. As shown, gastric residence system 200 comprises a central core and a plurality of arms extending radially from the central core. The dashed circle shown encircling the central core represents the approximate maximum opening size of the pylorus in an adult human (i.e., 20 mm). Gastric residence system 200 is designed to prevent premature passage through the pylorus when in an open configuration. As shown, the width (or diameter) of gastric residence system 200, as measured from the distal end of one arm, passing through the central core, and to the distal end of another arm, is at least twice that of the diameter of the pyloric opening.

Though the gastric residence system of FIG. 2 is sufficiently larger than the pyloric opening, it has been shown that it can bend into configurations small enough to prematurely pass through the pylorus. FIGS. 3A-3C show three different configurations that a gastric residence system may assume that can allow for premature passage through the pylorus. As shown in each Figure, the stiff arms of the gastric residence system remain straight. However, because the core of each of the gastric residence systems has a greater flexibility/elasticity than the stiff arms, the core can bend. The length of the stiff arm provides a lever arm that transfers forces of stomach contractions to the core. Longer stiff arms provide greater mechanical advantage and allow the core to bend under less force. The bending of the core can allow gastric residence systems having stiff arms to prematurely pass through the pylorus of a patient.

As shown in FIG. 3A, gastric residence system 302a is shown in a bended configuration having three stiff arms leading through the pyloric opening. FIG. 3B shows gastric residence system 302b in a bending configuration having two stiff arms leading through the pyloric opening. FIG. 3C shows gastric residence system 302c in a bended configuration analogous to the shape of a shuttlecock and having the core leading through the pyloric opening.

When a force is applied to an arm of a gastric residence system comprising relatively stiff material throughout the length of each arm, the force is transferred to the core of the gastric residence system. Because the core of the gastric residence system has a greater flexibility than the relatively stiff arms, the core bends or contorts under the force.

Accordingly, described herein are gastric residence systems comprising segments of arms having controlled stiffness. A gastric residence system comprising a first segment that is relatively stiffer than a second segment may help prevent gastric forces from being able to compress the gastric residence system into a configuration that may allow for premature passage through the pylorus. Arms, segments of arms, and gastric residence systems comprising arms and segments of arms are described in more detail with respect to the arms and coupling polymers of a gastric residence system.

### System Dimensions

The system must be able to adopt a compacted state with dimensions that enable the patient to swallow the system (or for the system to be introduced into the stomach by alternate means, such as a feeding tube or gastrostomy tube). Typically, the system is held in the compacted state by a container such as a capsule. Upon entry into the stomach, the system is then released from the container and adopts an uncompacted state, that is, an expanded conformation, with dimensions that prevent passage of the system through the pyloric sphincter, thus permitting retention of the system in the stomach.

Accordingly, the system should be capable of being placed inside a standard-sized capsule of the type commonly used in pharmacy. Standard capsule sizes in use in the United States are provided below in the Capsule Table below (see “Draft Guidance for Industry on Size, Shape, and Other Physical Attributes of Generic Tablets and Capsules” at URL www.regulations.gov/#!documentDetail; D=FDA-2013-N-1434-0002). As these are the outer dimensions of the capsule, and as dimensions will vary slightly between capsule manufacturers, the system should be capable of adopting a configuration which is about 0.5 to 1 mm smaller than the outer diameter shown, and about 1 to 2 mm shorter than the length shown in the Capsule Table.

Capsule Table

| Capsule Size | Outer Diameter (mm) | Length (mm) |
|---|---|---|
| 000 | 9.9 | 26.1 |
| 00 | 8.5 | 23.3 |
| 0 | 7.6 | 21.7 |
| 1 | 6.9 | 19.4 |
| 2 | 6.3 | 18.0 |
| 3 | 5.8 | 15.9 |
| 4 | 5.3 | 14.3 |
| 5 | 4.9 | 11.1 |

Capsules can be made of materials well-known in the art, such as gelatin or hydroxypropyl methylcellulose. In one embodiment, the capsule is made of a material that dissolves in the gastric environment, but not in the oral or esophageal environment, which prevents premature release of the system prior to reaching the stomach.

In one embodiment, the system will be folded or compressed into a compacted state in order to fit into the capsule, for example, in a manner such as that shown in FIG. 1B. Once the capsule dissolves in the stomach, the system will adopt a configuration suitable for gastric retention, for example, in a manner such as that shown in FIG. 1A. Preferred capsule sizes are 00 and 00el (a 00el-size capsule has the approximate length of a 000 capsule and the approximate width of a 00 capsule), which then places constraints on the length and diameter of the folded system.

Once released from the container, the system adopts an uncompacted state with dimensions suitable to prevent passage of the gastric residence system through the pyloric sphincter. In one embodiment, the system has at least two perpendicular dimensions, each of at least 2 cm in length; that is, the gastric residence system measures at least about 2 cm in length over at least two perpendicular directions. In another embodiment, the perimeter of the system in its uncompacted state, when projected onto a plane, has two perpendicular dimensions, each of at least 2 cm in length. The two perpendicular dimensions can independently have lengths of from about 2 cm to about 7 cm, about 2 cm to about 6 cm, about 2 cm to about 5 cm, about 2 cm to about 4 cm, about 2 cm to about 3 cm, about 3 cm to about 7 cm, about 3 cm to about 6 cm, about 3 cm to about 5 cm, about 3 cm to about 4 cm, about 4 cm to about 7 cm, about 4 cm to about 6 cm, about 4 cm to about 5 cm, or about 4 cm to about 4 cm. These dimensions prevent passage of the gastric residence system through the pyloric sphincter. For star-shaped polymers with N arms (where N is greater than or equal to three, such as N=6), the arms can have dimensions such that the system has at least two perpendicular dimensions, each of length as noted above. These two perpendicular dimensions are chosen as noted above in order to promote retention of the gastric residence system.

The system is designed to eventually break apart in the stomach at the end of the desired residence time (residence period), at which point the remaining components of the system are of dimensions that permit passage of the system through the pyloric sphincter, small intestine, and large intestine. Finally, the system is eliminated from the body by defecation, or by eventual complete dissolution of the system in the small and large intestines. Thus, coupling polymers or disintegrating matrices are placed in the gastric residence systems of the invention in a configuration such that, at the end of the desired residence period when the coupling polymers or disintegrating matrices break or dissolve, the uncoupled components of the gastric residence system have dimensions suitable for passage through the pyloric sphincter and elimination from the digestive tract.

### Residence Time

The residence time of the gastric residence system is defined as the time between administration of the system to the stomach and exit of the system from the stomach. In one embodiment, the gastric residence system has a residence time of about 24 hours, or up to about 24 hours. In one embodiment, the gastric residence system has a residence time of about 48 hours, or up to about 48 hours. In one embodiment, the gastric residence system has a residence time of about 72 hours, or up to about 72 hours. In one embodiment, the gastric residence system has a residence time of about 96 hours, or up to about 96 hours. In one embodiment, the gastric residence system has a residence time of about 5 days, or up to about 5 days. In one embodiment, the gastric residence system has a residence time of about 6 days, or up to about 6 days. In one embodiment, the gastric residence system has a residence time of about 7 days (about one week), or up to about 7 days (about one week). In one embodiment, the gastric residence system has a residence time of about 10 days, or up to about 10 days. In one embodiment, the gastric residence system has a residence time of about 14 days (about two weeks), or up to about 14 days (about two weeks).

In one embodiment, the gastric residence system has a residence time between about 24 hours and about 7 days. In one embodiment, the gastric residence system has a residence time between about 48 hours and about 7 days. In one embodiment, the gastric residence system has a residence time between about 72 hours and about 7 days. In one embodiment, the gastric residence system has a residence time between about 96 hours and about 7 days. In one embodiment, the gastric residence system has a residence time between about 5 days and about 7 days. In one embodiment, the gastric residence system has a residence time between about 6 days and about 7 days.

In one embodiment, the gastric residence system has a residence time between about 24 hours and about 10 days. In one embodiment, the gastric residence system has a residence time between about 48 hours and about 10 days. In one embodiment, the gastric residence system has a residence time between about 72 hours and about 10 days. In one embodiment, the gastric residence system has a residence time between about 96 hours and about 10 days. In one embodiment, the gastric residence system has a residence time between about 5 days and about 10 days. In one embodiment, the gastric residence system has a residence time between about 6 days and about 10 days. In one embodiment, the gastric residence system has a residence time between about 7 days and about 10 days.

In one embodiment, the gastric residence system has a residence time between about 24 hours and about 14 days. In one embodiment, the gastric residence system has a residence time between about 48 hours and about 14 days. In one embodiment, the gastric residence system has a residence time between about 72 hours and about 14 days. In one embodiment, the gastric residence system has a residence time between about 96 hours and about 14 days. In one embodiment, the gastric residence system has a residence time between about 5 days and about 14 days. In one embodiment, the gastric residence system has a residence time between about 6 days and about 14 days. In one embodiment, the gastric residence system has a residence time between about 7 days and about 14 days. In one embodiment, the gastric residence system has a residence time between about 10 days and about 14 days. The gastric residence system releases a therapeutically effective amount of agent (or salt thereof) during at least a portion of the residence time or residence period during which the system resides in the stomach. In one embodiment, the system releases a therapeutically effective amount of agent (or salt thereof) during at least about 25% of the residence time. In one embodiment, the system releases a therapeutically effective amount of agent (or salt thereof) during at least about 50% of the residence time. In one embodiment, the system releases a therapeutically effective amount of agent (or salt thereof) during at least about 60% of the residence time. In one embodiment, the system releases a therapeutically effective amount of agent (or salt thereof) during at least about 70% of the residence time. In one embodiment, the system releases a therapeutically effective amount of agent (or salt thereof) during at least about 75% of the residence time. In one embodiment, the system releases a therapeutically effective amount of agent (or salt thereof) during at least about 80% of the residence time. In one embodiment, the system releases a therapeutically effective amount of agent (or salt thereof) during at least about 85% of the residence time. In one embodiment, the system releases a therapeutically effective amount of agent (or salt thereof) during at least about 90% of the residence time. In one embodiment, the system releases a therapeutically effective amount of agent (or salt thereof) during at least about 95% of the residence time. In one embodiment, the system releases a therapeutically effective amount of agent (or salt thereof) during at least about 98% of the residence time. In one embodiment, the system releases a therapeutically effective amount of agent (or salt thereof) during at least about 99% of the residence time.

### Evaluation of Release Characteristics

The release characteristics of agent from segments, arms, and gastric residence systems can be evaluated by various assays. Assays for agent release are described in detail in the examples. Release of agent in vitro from segments, arms, and gastric residence systems can be measured by immersing a segment, arm, or gastric residence system in a liquid, such as water, 0.1N HCl, fasted state simulated gastric fluid (FaSSGF), or fed state simulated gastric fluid (FeSSGF). Fasted state simulated gastric fluid (FaSSGF) is preferred for release assays. Simulated gastric fluid indicates either fasted state simulated gastric fluid (FaSSGF) or fed state simulated gastric fluid (FeSSGF); when a limitation is specified as being measured in simulated gastric fluid (SGF), the limitation is met if the limitation holds in either fasted state simulated gastric fluid (FaSSGF) or fed state simulated gastric fluid (FeSSGF). For example, if a segment is indicated as releasing at least 10% of an agent over the first 24 hours in simulated gastric fluid, the limitation is met if the segment releases at least 10% of the agent over the first 24 hours in fasted state simulated gastric fluid, or if the segment releases at least 10% of the agent over the first 24 hours in fed state simulated gastric fluid.

Ethanol burst release is typically measured by immersing a segment, arm, or gastric residence system in a solution of 40% ethanol and 60% fasted state simulated gastric fluid for one hour, followed by immersing the same segment, arm, or gastric residence system in 100% fasted state simulated gastric fluid for the remainder of the test period, and measuring release of agent at appropriate time points. This test is designed to simulate the effects of consumption of alcoholic beverages by a patient having a gastric residence system of the invention deployed in the patient's stomach.

While in vitro tests can be performed using segments, arms, or gastric residence systems, use of segments for in vitro tests is most convenient for rapid evaluation of the release characteristics. When in vitro tests are done to compare release rates under different conditions (such as release in 100% FaSSGF versus release in 40% ethanol/60% FaSSGF), the comparison solutions are kept at the same temperature, such as room temperature, 25° C., or 37° C. Room temperature (ambient temperature) is a preferred temperature for comparisons; in one embodiment, the ambient temperature does not drop below 20° C. or exceed 25° C. (although it may fluctuate between 20° C. and 25° C.).

In vivo tests can be performed in animals such as dogs (for example, beagle dogs or hound dogs) and swine. For in vivo tests, a gastric residence system is used, since an individual segment or arm would not be retained in the stomach of the animal. Blood samples can be obtained at appropriate time points, and, if desired, gastric contents can be sampled by cannula or other technique.

Clinical trials in humans, conducted in accordance with appropriate laws, regulations, and institutional guidelines, also provide in vivo data.

### Gastric Delivery Pharmacokinetics for Gastric Residence Systems

The gastric residence systems of the invention provide for high bioavailability of the agent as measured by AUC_{inf} after administration of the systems, relative to the bioavailability of a conventional oral formulation of the agent. The systems also provide for maintenance of an approximately constant plasma level or a substantially constant plasma level of the agent.

Relative bioavailability, FREL, of two different formulations, formulation A and formulation B, is defined as:

F_{REL}=100×(AUC_{A}×Dose_{B})/(AUC_{B}×Dose_{A})

where AUC_{A} is the area under the curve for formulation A, AUC_{B} is the area under the curve for formulation B, Dose_{A} is the dosage of formulation A used, and Dose_{B} is the dosage of formulation B used. AUC, the area under the curve for the plot of agent plasma concentration versus time, is usually measured at the same time (t) after administration of each formulation, in order to provide the relative bioavailability of the formulations at the same time point. AUC_{inf} refers to the AUC measured or calculated over “infinite” time, that is, over a period of time starting with initial administration, and ending where the plasma level of the agent has dropped to a negligible amount.

In one embodiment, the substantially constant plasma level of agent provided by the gastric residence systems of the invention can range from at or above the trough level of the plasma level of agent when administered daily in a conventional oral formulation (that is, Cmin of agent administered daily in immediate-release formulation) to at or below the peak plasma level of agent when administered daily in a conventional oral formulation (that is, Cmax of agent administered daily in immediate-release formulation). In some embodiments, the substantially constant plasma level of agent provided by the gastric residence systems of the invention can be about 50% to about 90% of the peak plasma level of agent when administered daily in a conventional oral formulation (that is, Cmax of agent administered daily in immediate-release formulation). The substantially constant plasma level of agent provided by the gastric residence systems of the invention can be about 75% to about 125% of the average plasma level of agent when administered daily in a conventional oral formulation (that is, Cave of agent administered daily in immediate-release formulation). The substantially constant plasma level of agent provided by the gastric residence systems of the invention can be at or above the trough level of plasma level of agent when administered daily in a conventional oral formulation (that is, Cmin of agent administered daily in immediate-release formulation), such as about 100% to about 150% of Cmin.

The gastric residence systems of the invention can provide bioavailability of agent released from the system of at least about 50%, at least about 60%, at least about 70%, or at least about 80% of that provided by an immediate release form comprising the same amount of agent. As indicated above, the bioavailability is measured by the area under the plasma concentration-time curve (AUCinf).

### Dissolution Profile, Bioavailability and Pharmacokinetics for Gastric Residence Systems

Dissolution: The gastric residence systems described herein provide a steady release of an agent or a pharmaceutically acceptable salt thereof over an extended period of time. The systems are designed to release a therapeutically effective amount of an agent or salt thereof over the period of residence in the stomach. The release of agent (or salt thereof) can be measured in vitro or in vivo to establish the dissolution profile (elution profile, release rate) of the agent (or salt thereof) from a given residence system in a specific environment. The dissolution profile can be specified as a percentage of the original amount of agent (or salt thereof) present in the system which elutes from the system over a given time period.

Thus, in some embodiments, the agent (or salt thereof) contained in a gastric residence system can have a dissolution profile of 10-20% release between zero hours and 24 hours in a given environment. That is, over the 24-hour period after initial introduction of the gastric residence system into the environment of interest, 10-20% of the initial agent (or salt thereof) contained in the system elutes from the system.

The environment of interest can be 1) the stomach of a patient (that is, an in vivo environment), or 2) simulated gastric fluid (that is, an in vitro environment).

The gastric residence systems of the invention provide for high bioavailability of the agent (or salt thereof) as measured by AUCinf after administration of the systems, relative to the bioavailability of a conventional oral formulation of the agent (or salt thereof). The systems also provide for maintenance of a substantially constant plasma level of the agent (or salt thereof).

Parameters of interest for release include the linearity of release over the residence period of the gastric residence systems, the standard deviation of release over the residence period (which is related to linearity of release; a standard deviation of zero indicates that release is linear over the entire residence period), the release over the initial six hours of residence (that is, burst release upon initial administration), and total release of agent (or salt thereof) over the residence period. A preferable residence period is seven days, although other periods, such as two, three, four, five, six, eight, nine, ten, 11, 12, 13, or 14 days can be useful.

Linearity of agent (or salt thereof) release over the residence period refers to the amount released during each 24-hour period of residence. For a seven-day period of residence, it is desirable that about the amount of agent (or salt thereof) is released each day, i.e., that linearity of agent (or salt thereof) release is maximized. This will minimize the standard deviation of daily agent or agent salt release over the residence period. In some embodiments, the gastric release systems have a variation (or a standard deviation) for daily agent (or salt thereof) release of less than about 100%, less than about 90%, less than about 80%, less than about 70%, less than about 60%, less than about 50%, less than about 40%, less than about 30%, less than about 25%, less than about 20%, less than about 15%, less than about 10%, or less than about 5%, over the period of residence. In some embodiments, the period of residence can be about three days, about seven days, about ten days, or about two weeks.

Minimization of burst release, that is, release over the initial period of residence (such as six hours, twelve hours, or 24 hours after administration of a gastric residence system) is desirable in order to maintain a predictable and steady release profile. If T is the total agent (or salt thereof) release over the residence period (in units of mass), and D is the number of days of the residence period, then completely linear release would mean that about T/D mass of agent (or salt thereof) is released per day. If the period over which burst release is measured is the first six hours, then a linear release profile will result in 0.25×T/D mass of agent (or salt thereof) released during the first six hours. In percentage terms of the total amount of agent (or salt thereof) released over the residence period of D days, linear release would be about 100/D % of agent (or salt thereof) per day, and a linear release over the first six hours would be 25/D %. (Note that 100% in this context indicates the total amount of agent (or salt thereof) released, regardless of how much agent (or salt thereof) is contained in the initial formulation.) Thus, for a seven day residence period, linear release over the first six hours would be about 3.6% of the total amount of agent (or salt thereof) released over the seven-day period.

In some embodiments, during the initial six hours of residence after administration the gastric residence systems release about 0.2 to about 2 times T/D of the total mass of agent (or salt thereof) T released over the residence period of D days, or about 0.2 to about 1.75 times T/D of the total mass of agent (or salt thereof) T released over the residence period of D days, or about 0.2 to about 1.5 times T/D of the total mass of agent (or salt thereof) T released over the residence period of D days, or about 0.2 to about 1.25 times T/D of the total mass of agent (or salt thereof) T released over the residence period of D days, or about 0.2 to about 1 times T/D of the total mass of agent (or salt thereof) T released over the residence period of D days, or about 0.2 to about 0.8 times T/D of the total mass of agent (or salt thereof) T released over the residence period of D days, or about 0.2 to about 0.75 times T/D, or about 0.2 to about 0.7 times T/D, or about 0.2 to about 0.6 times T/D, or about 0.2 to about 0.5 times T/D, or about 0.2 to about 0.4 times T/D, or about 0.2 to about 0.3 times T/D, or about 0.25 to about 2 times T/D, or about 0.3 to about 2 times T/D, or about 0.4 to about 2 times T/D, or about 0.5 to about 2 times T/D, or about 0.6 to about 2 times T/D, or about 0.7 to about 2 times T/D, or about 0.25 to about 1.5 times T/D, or about 0.3 to about 1.5 times T/D, or about 0.4 to about 1.5 times T/D, or about 0.5 to about 1.5 times T/D, or about 0.6 to about 1.5 times T/D, or about 0.7 to about 1.5 times T/D, or about 0.25 to about 1.25 times T/D, or about 0.3 to about 1.25 times T/D, or about 0.4 to about 1.25 times T/D, or about 0.5 to about 1.25 times T/D, or about 0.6 to about 1.25 times T/D, or about 0.7 to about 1.25 times T/D, or about 0.25 to about 1 times T/D, or about 0.3 to about 1 times T/D, or about 0.4 to about 1 times T/D, or about 0.5 to about 1 times T/D, or about 0.6 to about 1 times T/D, or about 0.7 to about 1 times T/D, or about 0.25 times T/D, or about 0.25 to about 0.8 times T/D, or about 0.3 to about 0.8 times T/D, or about 0.4 to about 0.8 times T/D, or about 0.5 to about 0.8 times T/D, or about 0.6 to about 0.8 times T/D, or about 0.7 to about 0.8 times T/D, or about 0.8 times T/D, about 1 times T/D, about 1.25 times T/D, about 1.5 times T/D, or about 2 times T/D.

In some embodiment of the gastric residence systems, during the initial six hours of residence after administration the gastric residence systems release about 2% to about 10% of the total mass of agent (or salt thereof) released over the residence period, or about 3% to about 10%, or about 4% to about 10%, or about 5% to about 10%, or about 6% to about 10%, or about 7% to about 10%, or about 8% to about 10%, or about 9% to about 10%, or about 2% to about 9%, or about 2% to about 8%, or about 2% to about 7%, or about 2% to about 6%, or about 2% to about 5%, or about 2% to about 4%, or about 2% to about 3%.

In some embodiments of the gastric residence systems, where the gastric residence systems have a residence period of about seven days, during the initial six hours of residence after administration the gastric residence systems release about 2% to about 10% of the total mass of agent (or salt thereof) released over the residence period of seven days, or about 3% to about 10%, or about 4% to about 10%, or about 5% to about 10%, or about 6% to about 10%, or about 7% to about 10%, or about 8% to about 10%, or about 9% to about 10%, or about 2% to about 9%, or about 2% to about 8%, or about 2% to about 7%, or about 2% to about 6%, or about 2% to about 5%, or about 2% to about 4%, or about 2% to about 3%.

In some embodiments, during the initial 24 hours of residence after administration, the gastric residence systems release about 10% to about 35% of the total mass of agent (or salt thereof) released over the residence period, or about 10% to about 30%, or about 10% to about 25%, or about 10% to about 20%, or about 10% to about 15%, or about 15% to about 35%, or about 15% to about 35%, or about 15% to about 30%, or about 20% to about 30%, or about 25% to about 35%, or about 25% to about 30%, or about 30% to about 35%.

In some embodiments, where the gastric residence systems have a residence period of about seven days, during the initial 24 hours of residence after administration the gastric residence systems release about 10% to about 35% of the total mass of agent (or salt thereof) released over the residence period of seven days, or about 10% to about 30%, or about 10% to about 25%, or about 10% to about 20%, or about 10% to about 15%, or about 15% to about 35%, or about 15% to about 35%, or about 15% to about 30%, or about 20% to about 30%, or about 25% to about 35%, or about 25% to about 30%, or about 30% to about 35%.

### Elastomers

Elastomers (also referred to as elastic polymers or tensile polymers) enable the gastric residence system to be compacted, such as by being folded or compressed, into a form suitable for administration to the stomach by swallowing a container or capsule containing the compacted system. Upon dissolution of the capsule in the stomach, the gastric residence system expands into a shape which prevents passage of the system through the pyloric sphincter of the patient for the desired residence time of the system. Thus, the elastomer must be capable of being stored in a compacted configuration in a capsule for a reasonable shelf life, and of expanding to its original shape, or approximately its original shape, upon release from the capsule. In one embodiment, the elastomer is a silicone elastomer. In one embodiment, the elastomer is formed from a liquid silicone rubber (LSR), such as sold in the Dow Corning QP-1 liquid silicone rubber kit. In one embodiment, the elastomer is crosslinked polycaprolactone. In one embodiment, the elastomer is an enteric polymer, such as those listed in the Enteric Polymer Table. In some embodiments, the coupling polymer(s) used in the system are also elastomers. Elastomers are preferred for use as the central polymer in the star-shaped or stellate design of the gastric residence systems.

In one embodiment, both the coupling polymer and elastomer are enteric polymers, which provides for more complete breakage of the system into the carrier polymer-agent pieces if the system enters the intestine, or if the patient drinks a mildly basic solution in order to induce passage of the system.

Examples of elastomers which can be used include silicones, such as those formed using Dow Corning QP-1 kits; urethane-cross-linked polycaprolactones; poly(acryloyl 6-aminocaproic acid) (PA6ACA); poly(methacrylic acid-co-ethyl acrylate) (EUDRAGIT L 100-55); and mixtures of poly(acryloyl 6-aminocaproic acid) (PA6ACA) and poly(methacrylic acid-co-ethyl acrylate) (EUDRAGIT L 100-55).

Flexible coupling polymers, i.e., elastomeric coupling polymers or elastomers, are used as the central polymer in the star-shaped or stellate design of the gastric residence systems. A particularly preferred elastomer for use as the central elastomer of the stellate or star configuration is silicone rubber. Liquid silicone rubber (LSR) can be molded easily and cured into a desired shape. The Dow Corning QP-1 series, comprising cross-linked dimethyl and methyl-vinyl siloxane copolymers and reinforcing silica, are examples of such silicone rubber polymers (see, for example, the Web site www.dowcoming.com/DataFiles/090276fe8018ed07.pdf). Non-segmented arms or arms comprising segments of carrier polymer-agent components can then be attached to the central silicone rubber elastomer. Another elastomer which can be used as the central elastomer in the stellate design is crosslinked polycaprolactone.

Specific configurations of gastric residence systems are disclosed in International Patent Application No. WO 2017/100367, and any of those configurations can be used for the gastric residence systems disclosed herein.

### Carrier Polymers for Segments and Arms (Carrier Polymer-Agent Component)

The segments and arms of the gastric residence system comprise a carrier polymer-agent component, which comprises the agent (or a pharmaceutically acceptable salt of an agent) to be eluted from the gastric residence system in the gastric environment. The agent is blended into the carrier polymer to form a carrier polymer-agent mixture. This mixture can be formed into the desired shape or shapes for use as carrier polymer-agent components in the systems. After the drug or drug salt is blended into the carrier polymer to form the carrier polymer-drug mixture, the drug or drug salt is distributed or dispersed throughout the blended mixture. If excipients, anti-oxidants, or other ingredients are included in the carrier polymer-drug blend, they will also be distributed or dispersed throughout the blended mixture.

Selection of the carrier material for the agent or pharmaceutically acceptable salt thereof in a gastric residence system influences the release profile of drug during the period of gastric residence. Carrier polymers may be thermoplastic, to allow extrusion using hot melt extrusion or 3D printing techniques. They may also have a high enough melt strength and viscosity to enable extrusion into the required geometry. They may have low melting temperatures (for example, less than about 120° C.), to avoid exposing agents or drugs to high temperatures during manufacture. They may have a sufficient mechanical strength (Young's modulus, compression strength, tensile strength) to avoid breaking in the stomach during the desired residence period. Further, they should be capable of forming stable blends with agents, therapeutic agents, drugs, excipients, dispersants, and other additives.

Exemplary carrier polymers suitable for use in this invention include, but are not limited to, hydrophilic cellulose derivatives (such as hydroxypropylmethyl cellulose, hydroxypropyl cellulose, hydroxymethyl cellulose, hydroxyethyl cellulose, carboxymethylcellulose, sodium-carboxymethylcellulose), cellulose acetate phthalate, poly(vinyl pyrrolidone), ethylene/vinyl alcohol copolymer, poly(vinyl alcohol), carboxyvinyl polymer (Carbomer), Carbopol® acidic carboxy polymer, polycarbophil, poly(ethyleneoxide) (Polyox WSR), polysaccharides and their derivatives, polyalkylene oxides, polyethylene glycols, chitosan, alginates, pectins, acacia, tragacanth, guar gum, locust bean gum, vinylpyrrolidonevinyl acetate copolymer, dextrans, natural gum, agar, agarose, sodium alginate, carrageenan, fucoidan, furcellaran, laminaran, hypnea, eucheuma, gum arabic, gum ghatti, gum karaya, arbinoglactan, amylopectin, gelatin, gellan, hyaluronic acid, pullulan, scleroglucan, xanthan, xyloglucan, maleic anhydride copolymers, ethylenemaleic anhydride copolymer, poly(hydroxyethyl methacrylate), ammoniomethacrylate copolymers (such as Eudragit RL or Eudragit RS), poly(ethylacrylate-methylmethacrylate) (Eudragit NE), Eudragit E (cationic copolymer based on dimethylamino ethyl methylacrylate and neutral methylacrylic acid esters), poly(acrylic acid), polymethacrylates/polyethacrylates such as poly(methacrylic acid), methylmethacrylates, and ethyl acrylates, polylactones such as poly(caprolactone), polyanhydrides such as poly[bis-(p-carboxyphenoxy)-propane anhydride], poly(terephthalic acid anhydride), polypeptides such as polylysine, polyglutamic acid, poly(ortho esters) such as copolymers of DETOSU with diols such as hexane diol, decane diol, cyclohexanedimethanol, ethylene glycol, polyethylene glycol and incorporated herein by reference those poly(ortho) esters described and disclosed in U.S. Pat. No. 4,304,767, starch, in particular pregelatinized starch, and starch-based polymers, carbomer, maltodextrins, amylomaltodextrins, dextrans, poly(2-ethyl-2-oxazoline), poly(ethyleneimine), polyurethane, poly(lactic acid), poly(glycolic acid), poly(lactic-co-glycolic acid) (PLGA), polyhydroxyalkanoates, polyhydroxybutyrate, and copolymers, mixtures, blends and combinations thereof. Polycaprolactone (PCL) is a preferred carrier polymer. In some embodiments, polydioxanone is used as the carrier polymer. In any of the embodiments of the gastric residence system, the carrier polymer used in the gastric residence system can comprise polycaprolactone, such as linear polycaprolactone with a number-average molecular weight (Mn) range between about 60 kiloDalton (kDa) to about 100 kDa; 75 kDa to 85 kDa; or about 80 kDa; or between about 45 kDa to about 55 kDa; or between about 50 kDa to about 110,000 kDa, or between about 80 kDa to about 110,000 kDa.

Further, release of drug can be modulated by a wide variety of excipients included in the carrier polymer-agent component. Soluble excipients include P407, Eudragit E, PEG, Polyvinylpyrrolidone (PVP), and Polyvinyl alcohol (PVA). Insoluble, wicking excipients include Eudragit RS and Eudragit RL. Degradable excipients include PLA, PLGA, PLA-PCL, polydioxanone, and linear copolymers of caprolactone and glycolide; polyaxial block copolymers of glycolide, caprolactone, and trimethylene carbonate; polyaxial block copolymers of glycolide, trimethylene carbonate, and lactide; polyaxial block copolymers of glycolide, trimethylene carbonate and polypropylene succinate; polyaxial block copolymers of caprolactone, lactide, glycolide, and trimethylene carbonate; polyaxial block copolymers of glycolide, trimethylene carbonate, and caprolactone; and linear block copolymers of lactide, caprolactone, and trimethylene carbonate; such as linear copolymers of caprolactone (95%) and glycolide (5%); polyaxial block copolymers of glycolide (68%), caprolactone (29%), and trimethylene carbonate (3%); polyaxial block copolymers of glycolide (86%), trimethylene carbonate (9%), and lactide (5%); polyaxial block copolymers of glycolide (70%), trimethylene carbonate (27%) and polypropylene succinate (2%); polyaxial block copolymers of caprolactone (35%), lactide (34%), glycolide (17%), and trimethylene carbonate (14%); polyaxial block copolymers of glycolide (55%), trimethylene carbonate (25%), and caprolactone (20%); and linear block copolymers of lactide (39%), caprolactone (33%), and trimethylene carbonate (28%). Insoluble, swellable excipients include Polyvinyl acetate (PVAc), Crospovidone, Croscarmellose, HPMCAS, and linear block copolymers of dioxanone and ethylene glycol; linear block copolymers of lactide and ethylene glycol; linear block copolymers of lactide, ethylene glycol, trimethyl carbonate, and caprolactone; linear block copolymers of lactide, glycolide, and ethylene glycol; linear block copolymers of glycolide, polyethylene glycol, and ethylene glycol; such as linear block copolymers of dioxanone (80%) and ethylene glycol (20%); linear block copolymers of lactide (60%) and ethylene glycol (40%); linear block copolymers of lactide (68%), ethylene glycol (20%), trimethyl carbonate (10%), and caprolactone (2%); linear block copolymers of lactide (88%), glycolide (8%), and ethylene glycol (4%); linear block copolymers of glycolide (67%), polyethylene glycol (28%), and ethylene glycol (5%). Surfactants include Lecithin, Taurocholate, SDS, Soluplus, Fatty acids, and Kolliphor RH40.

Other excipients can be added to the carrier polymers to modulate the release of agent. Such excipients can be added in amounts from about 1% to 15%, preferably from about 5% to 10%, more preferably about 5% or about 10%. Examples of such excipients include Poloxamer 407 (available as Kolliphor P407, Sigma Cat #62035), poly(ethylene glycol)-block-poly(propylene glycol)-block-poly(ethylene glycol), CAS No. 9003-11-6; H—(OCH2CH2)x-(O—CH(CH3)CH2)y-(OCH2CH2)z-OH where x and z are about 101 and y is about 56); Pluronic P407; Eudragit E, Eudragit EPO (available from Evonik); hypromellose (available from Sigma, Cat #H3785), Kolliphor RH40 (available from Sigma, Cat #07076), polyvinyl caprolactam, polyvinyl acetate (PVAc), polyvinylpyrrolidone (PVP), polyvinyl alcohol (PVA), polyethylene glycol (PEG), and Soluplus (available from BASF; a copolymer of polyvinyl caprolactam, polyvinyl acetate, and polyethylene glycol). Preferred soluble excipients include Eudragit E, polyethylene glycol (PEG), polyvinylpyrrolidone (PVP), polyvinyl acetate (PVAc), and polyvinyl alcohol (PVA). Preferred insoluble excipients include Eudragit RS and Eudragit RL. Preferred insoluble, swellable excipients include crospovidone, croscarmellose, hypromellose acetate succinate (HPMCAS), and carbopol. EUDRAGIT RS and EUDRAGIT RL are registered trademarks of Evonik (Darmstadt, Germany) for copolymers of ethyl acrylate, methyl methacrylate and methacrylic acid ester with quaternary ammonium groups (trimethylammonioethyl methacrylate chloride), having a molar ratio of ethyl acrylate, methyl methacrylate and trimethylammonioethyl methacrylate of about 1:2:0.2 in Eudragit® RL and about 1:2:0.1 in Eudragit® RS. Preferred insoluble, swellable excipients include crospovidone, croscarmellose, hypromellose acetate succinate (HPMCAS), carbopol, and linear block copolymers of dioxanone and ethylene glycol; linear block copolymers of lactide and ethylene glycol; linear block copolymers of lactide, ethylene glycol, trimethyl carbonate, and caprolactone; linear block copolymers of lactide, glycolide, and ethylene glycol; linear block copolymers of glycolide, polyethylene glycol, and ethylene glycol; such as linear block copolymers of dioxanone (80%) and ethylene glycol (20%); linear block copolymers of lactide (60%) and ethylene glycol (40%); linear block copolymers of lactide (68%), ethylene glycol (20%), trimethyl carbonate (10%), and caprolactone (2%); linear block copolymers of lactide (88%), glycolide (8%), and ethylene glycol (4%); linear block copolymers of glycolide (67%), polyethylene glycol (28%), and ethylene glycol (5%).

Further examples of excipients that can be used in the segments of the gastric residence system are listed in the Excipient Table below.

Excipient Table

| Function | General examples | Specific examples |
|---|---|---|
| Polymeric and non-polymeric | Polyalkylene oxides | Kolliphor RH, Kolliphor P407, |
| solubilizers | Polyethoxylated castor oil | Soluplus, Cremophor, SDS |
| | Detergents | |
| Release-enhancing excipient | Acrylate polymers | Eudragit RL |
| (porogen or wicking agent) | Acrylate co-polymers | Eudragit RS |
| | Polyvinylpyrrolidone | Eudragit E |
| | | Linear block copolymer of |
| | | dioxanone and ethylene glycol |
| | | (e.g., 80:20 ratio) |
| Dispersant | porous inorganic material | silica, hydrophilic-fumed silica, |
| | polar inorganic material | hydrophobic colloidal silica, |
| | non-toxic metal oxides | magnesium aluminum silicate, |
| | amphiphilic organic molecules | stearate salts, calcium stearate, |
| | polysaccharides, cellulose, | magnesium stearate, |
| | cellulose derivatives | microcrystalline cellulose, |
| | fatty acids | carboxymethylcellulose, |
| | detergents | hypromellose, phospholipids, |
| | | polyoxyethylene stearates, zinc |
| | | acetate, alginic acid, lecithin, |
| | | sodium lauryl sulfate, aluminum |
| | | oxide |
| Stabilizer/Preservative agent | Anti-oxidants | Tocopherols |
| | Anti-microbial agents | Alpha-tocopherol |
| | Buffering substances/pH | Ascorbic acid; ascorbate salts |
| | stabilizers | Carotenes |
| | | Butylated hydroxytoluene (BHT) |
| | | Butylated hydroxyanisole (BHA) |
| | | Fumaric acid |
| | | calcium carbonate |
| | | calcium lactate |
| | | calcium phosphate |
| | | sodium phosphate |
| | | sodium bicarbonate |

### Controlling the Stiffness of Arms of a Gastric Residence System

Described herein are gastric residence systems having controlled stiffness. By controlling the stiffness of an element of a gastric residence system that widens/enlarges the device to its open configuration (such as an arm), the risk of premature passage of the gastric residence system through the pylorus may be minimized. Accordingly, gastric residence systems having arms of controlled stiffness described herein can help improve the efficacy and reliability of gastric residence systems. Additionally, gastric residence systems having arms of controlled stiffness as described herein can help prevent the gastric residence system from bending into configurations that allow for premature passage through the pylorus.

Gastric residence systems having arms of controlled stiffness require more force for the gastric residence system to bend into configurations suitable for premature passage through the pylorus. Described herein are gastric residence systems having controlled stiffness of any member that can widen or enlarge the gastric residence system into its open configuration (such as an arm) to help minimize the risk of the gastric residence system passing through the pylorus of a patient prematurely.

As described herein, a gastric residence system having arms of a controlled stiffness is defined as a system comprising one or more arms having at least a portion of the arm made of a flexible material. In some embodiments, one or more arms may include a first segment comprising a first polymer composition and a second segment comprising a second polymer composition, wherein the second segment is more flexible than the first segment.

In some embodiments, the one or more arms extend radially. A proximal end of the one or more arms may be connected to a core. In some embodiments, a gastric residence system may include a plurality of arms extending radially. In some embodiments, a gastric residence system may include a plurality of arms connected to a core at the proximal end of each arm, the plurality of arms extending radially from the core. In some embodiments, a gastric residence system may comprise a plurality of arms, each arm comprising a first segment and a second segment.

The first polymer composition of a flexible arm of a gastric residence system disclosed herein may comprise a relatively stiff polymer. For example, suitable polymers may include polycaprolactone, polylactic acid, poly(lactic-co-glycolic acid), HPMCAS, high durometer TPU, and/or combinations thereof. Other examples may include hydrophilic cellulose derivatives (such as hydroxypropylmethyl cellulose, hydroxypropyl cellulose, hydroxymethyl cellulose, hydroxyethyl cellulose, carboxymethylcellulose, sodium-carboxymethylcellulose), cellulose acetate phthalate, poly(vinyl pyrrolidone), ethylene/vinyl alcohol copolymer, poly(vinyl alcohol), carboxyvinyl polymer (Carbomer), Carbopol® acidic carboxy polymer, polycarbophil, poly(ethyleneoxide) (Polyox WSR), polysaccharides and their derivatives, polyalkylene oxides, polyethylene glycols, chitosan, alginates, pectins, acacia, tragacanth, guar gum, locust bean gum, vinylpyrrolidonevinyl acetate copolymer, dextrans, natural gum, agar, agarose, sodium alginate, carrageenan, fucoidan, furcellaran, laminaran, hypnea, eucheuma, gum arabic, gum ghatti, gum karaya, arbinoglactan, amylopectin, gelatin, gellan, hyaluronic acid, pullulan, scleroglucan, xanthan, xyloglucan, maleic anhydride copolymers, ethylenemaleic anhydride copolymer, poly(hydroxyethyl methacrylate), ammoniomethacrylate copolymers (such as Eudragit RL or Eudragit RS), poly(ethylacrylate-methylmethacrylate) (Eudragit NE), Eudragit E (cationic copolymer based on dimethylamino ethyl methylacrylate and neutral methylacrylic acid esters), poly(acrylic acid), polymethacrylates/polyethacrylates such as poly(methacrylic acid), methylmethacrylates, and ethyl acrylates, polylactones such as poly(caprolactone), polyanhydrides such as poly[bis-(p-carboxyphenoxy)-propane anhydride], poly(terephthalic acid anhydride), polypeptides such as polylysine, polyglutamic acid, poly(ortho esters) such as copolymers of DETOSU with diols such as hexane diol, decane diol, cyclohexanedimethanol, ethylene glycol, polyethylene glycol and incorporated herein by reference those poly(ortho) esters described and disclosed in U.S. Pat. No. 4,304,767, starch, in particular pregelatinized starch, and starch-based polymers, carbomer, maltodextrins, amylomaltodextrins, dextrans, poly(2-ethyl-2-oxazoline), poly(ethyleneimine), polyurethane, poly(lactic acid), poly(glycolic acid), poly(lactic-co-glycolic acid) (PLGA), polyhydroxyalkanoates, polyhydroxybutyrate, and copolymers, mixtures, blends and combinations thereof. In some embodiments, the first segment may also comprise one or more therapeutic agent or active pharmaceutical ingredients (APIs).

In some embodiments, the first polymer composition may comprise 10-90 wt. % or 50-70 wt. % polycaprolactone. In some embodiments, the first polymer composition may comprise less than 90 wt. %, less than 80 wt. %, less than 70 wt. %, less than 60 wt. %, less than 50 wt. %, less than 40 wt. %, less than 30 wt. %, or less than 20 wt. % polycaprolactone. In some embodiments, the first polymer composition may include more than 20 wt. %, more than 30 wt. %, more than 40 wt. %, more than 50 wt. %, more than 60 wt. %, more than 70 wt. %, or more than 80 wt. % polycaprolactone.

In some embodiments, the first polymer composition may comprise 10-90 wt. % or 30-70 wt. % therapeutic agent or API. In some embodiments, the first polymer composition may comprise less than 90 wt. %, less than 80 wt. %, less than 70 wt. %, less than 60 wt. %, less than 50 wt. %, less than 40 wt. %, less than 30 wt. %, or less than 20 wt. % therapeutic agent or API. In some embodiments, the first polymer composition may include more than 20 wt. %, more than 30 wt. %, more than 40 wt. %, more than 50 wt. %, more than 60 wt. %, more than 70 wt. %, or more than 80 wt. % therapeutic agent or API.

The second polymer composition of an arm of a gastric residence system disclosed herein may comprise a primary polymer that is flexible relative to the polymer of the first polymer composition. For example, suitable relatively “flexible” polymers may include one or more of a polyurethane, a polyether-polyamide copolymer, a thermoplastic elastomer, a thermoplastic polyurethane, polycaprolactone polylactic acid copolymer, a poly(trimethylene carbonate), a polyglycerol sebacate, a polyethylene-co-vinyl acetate, and a silicone. In some embodiments, the second polymer composition of an arm may actually comprise the same primary polymer as the first polymer composition. For example, the second polymer composition may comprise polycaprolactone. However, unlike the first polymer composition, the second polymer composition may additionally comprise a soluble material (e.g., copovidone, poloxamers). Thus, upon hydration (e.g., within the stomach), the second polymer composition will soften such that the stiffness of the second polymer composition of the second segment is less than the first polymer composition of the first segment. Suitable commercially-available polymers may include Pathway™ TPU polymers (The Lubrizol Corporation), Tecoflex™ (The Lubrizol Corporation), Tecophilic™ (The Lubrizol Corporation), Carbothane™ (The Lubrizol Corporation), Isoplast® (The Lubrizol Corporation), Pebax® (Arkema), Texin® (Covestro), Chronoflex (AdvanSource Biomaterials), NEUSoft™ (PolyOne), and Medalist® TPEs (Teknor Apex). Additional polymers include thermoplastic polyurethanes, polyether polyamides, vinyl acetates, styrenics, thermoplastic silicone copolymers, thermoplastic vulcanizates, liquid silicone rubber, poly(ethylene-co-vinyl acetate), and bioresorbable polymers. Bioresorbable polymers include, but are not limited to, polycaprolactone-polygylicolide copolymer, poly(glycerol sebacate), and poly(glycerol sebacate) polyurethane.

In some embodiments, the second polymer composition may comprise 10-90 wt. % or 40-70 wt. % primary polymer. In some embodiments, the second polymer composition may comprise less than 90 wt. %, less than 80 wt. %, less than 70 wt. %, less than 60 wt. %, less than 50 wt. %, less than 40 wt. %, less than 30 wt. %, or less than 20 wt. % primary polymer. In some embodiments, the second polymer composition may include more than 20 wt. %, more than 30 wt. %, more than 40 wt. %, more than 50 wt. %, more than 60 wt. %, more than 70 wt. %, or more than 80 wt. % primary polymer.

In some embodiments, the second polymer composition may additionally include one or more water-soluble excipients (which may include one or more polymers to the primary polymer described previously). For example, suitable water-soluble excipients may include a copovidone, a poloxamer, and/or a polyethylene oxide. Suitable commercially-available water-soluble excipients can include Kolliphor P407 (poloxamer 407, poly(ethylene glycol)-block-poly(propylene glycol)-block-poly(ethylene glycol)), PEG-PCL, SIF (FaSSIF/FaSSGF powder from BioRelevant), EPO (dimethylaminoethyl methacrylate-butyl methacrylate-methyl methacrylate copolymer), Kollidon VA64 (vinylpyrrolidone-vinyl acetate copolymer in a ratio of 6:4 by mass), polyvinyl pyrrolidine.

The second polymer composition may comprise 5-70 wt. % or 10-40 wt. % water-soluble excipients. In some embodiments, the second polymer composition may comprise less than 70 wt. %, less than 60 wt. %, less than 50 wt. %, less than 40 wt. %, less than 30 wt. %, less than 20 wt. %, or less than 10 wt. % water-soluble excipients. In some embodiments, the second polymer composition may comprise more than 5 wt. %, more than 10 wt. %, more than 20 wt. %, more than 30 wt. %, more than 40 wt. %, more than 50 wt. %, or more than 60 wt. % water-soluble excipients.

In some embodiments, the second polymer composition may comprise additional excipients. For example, the second polymer composition may comprise bismuth subcarbonate, silica, vitamin E succinate, iron oxide, a polyethylene glycol, polyvinyl acetate and polyvinylcaprolactame-based graft copolymer (Soluplus®), sodium starch glycolate, and/or hydroxypropyl cellulose. In some embodiments, the second polymer composition may comprise 10-70 wt. % or 20-50 wt. % excipients. In some embodiments, the second polymer composition may comprise less than 70 wt. %, less than 60 wt. %, less than 50 wt. %, less than 40 wt. %, less than 30 wt. %, or less than 20 wt. % excipients. In some embodiments, the second polymer composition may comprise more than 10 wt. %, more than 20 wt. %, more than 30 wt. %, more than 40 wt. %, more than 50 wt. %, or more than 60 wt. % excipients.

In some embodiments, the second polymer composition may additionally comprise a therapeutic agent or API. The second polymer composition may comprise 20-80 wt. % or 40-60 wt. % Therapeutic agent or API. In some embodiments, the second polymer composition may comprise less than 80 wt. %, less than 70 wt. %, less than 60 wt. %, less than 50 wt. %, less than 40 wt. %, or less than 30 wt. % therapeutic agent or API. In some embodiments, the second polymer composition may comprise more than 20 wt. %, more than 30 wt. %, more than 40 wt. %, more than 50 wt. %, more than 60 wt. %, or more than 70 wt. % therapeutic agent or API.

Some polymer materials that are useful for creating arms of controlled stiffness may have an added advantage in thermal stability. For example, gastric residence systems may experience temperature variation during shipping and distribution. Shipping data suggest that cargo temperature extremes may approach 60° C. in some climates (Singh et al, Packag. Technol. Sci. 2012; 25: 149-160). The polymers that comprise gastric residence systems should be physically stable at this temperature if they are to be shipped without cold chain packaging and storage.

Polycaprolactone is a preferred polymer for relatively stiff arms (or stiff/first segments), and thermoplastic polyurethane is a preferred polymer for creating arms of controlled stiffness (i.e., second segments). Polycaprolactone-based arms are physically stable when exposed to temperatures as high as 55° C., but melt if they reach 60° C. When stored in a capsule, arms that begin to melt can adhere to one another and prevent the gastric residence system from unfolding in the stomach. Thermoplastic polyurethanes such as Pathway PY-PT72AE provide improved thermal stability. Pathway PY-PT72AE is an amorphous material that does not undergo a clear melt transition but does soften at elevated temperatures.

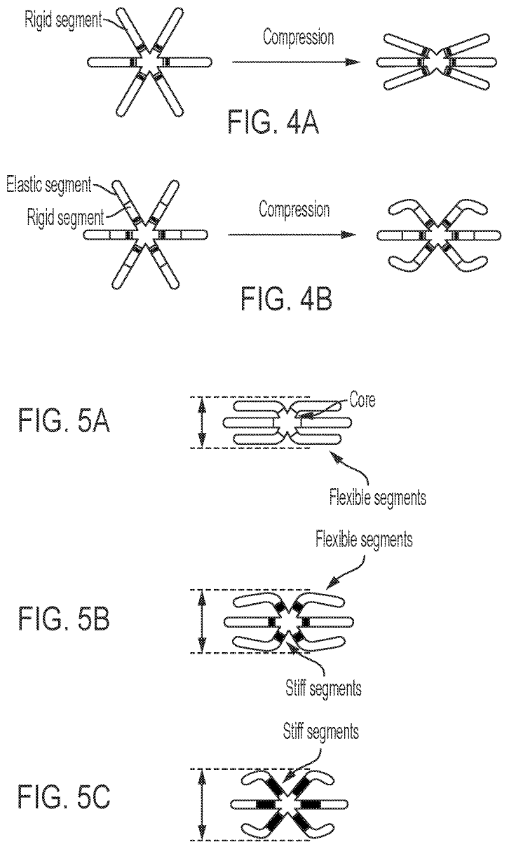

FIGS. 4A and 4B show the response of a gastric residence system comprising relatively stiff arms compared to a gastric residence system comprising relatively flexible arms (as disclosed herein) when subjected to a radial force compression test. Gastric residence system 402a comprises relatively stiff arms. When compressed, the compression force is transferred to the more flexible core of gastric residence system 402a, resulting in a gastric residence system in a bended configuration that is capable of passing through the pylorus of a patient (i.e., an opening having a diameter of 20 mm).

Conversely, FIG. 4B shows the behavior of gastric residence system 402b (having relatively flexible arms) when subjected to a radial force compression test. First segment 404, at a proximal end, is stiffer than second segment 406, at a distal end, of each arm. As shown in the figure, when compressed, second segment 406 absorbs some of the compression force. Thus, the compression forces are not transferred to the core of gastric residence system 402b as is the case with gastric residence system 402a of FIG. 4A. To compress the stiff inner segments of the arms to pyloric size, greater force is required due to the shorter lever arm attached to the flexible core. This can mean that gastric residence system 402b requires a greater compression force to bend it into a configuration small enough to pass through the pylorus of a patient (i.e., an opening having a diameter of 20 mm). Accordingly, gastric residence system 402b can more easily resist premature passage through the pylorus of a patient than gastric residence system 402a.

FIGS. 5A-5C show various configurations of gastric residence systems described herein. In particular, the relative sizes of the first segment compared to the second segment of a flexible arm may be varied. As shown in the figures, as the second segment increases, so too does the compression force required to compress the gastric residence system into a bended configuration small enough to pass through a pylorus (i.e., an opening having a diameter of 20 mm). (As long as the size of the stiff inner portion and the core is still larger than the diameter of the pylorus.) It may be assumed that the compression force applied to each gastric residence system of FIGS. 5A, 5B, and 5C is the same.

FIG. 5A shows gastric residence system 502a having arms comprising relatively flexible material the full length of each arm (e.g., arms comprising only a second segment, and no first segment). As shown, the compression force applied to gastric residence system 502a compresses the system to the shortest height of the three gastric residence systems depicted in FIGS. 5A-5C. Gastric residence system 502a will more easily pass through the pylorus than a stellate with fully stiff arms (i.e., comprising only a first segment). Thus, arms having only a second, flexible material are not effective at preventing premature passage through the pylorus.

The benefit of the second, relatively flexible, portion comes in when the innermost sections of the arms are relatively stiff. The second segment of the arms bends relatively easily, but more force is required to compress the inner first segments. If the stiff sections are too short, the bending of the second segments will make the gastric residence system small enough to pass through the pylorus.

FIG. 5B shows gastric residence system 502b having a first segment 504b and a second segment 506b. As shown, the second segment is larger than the first segment. The second segment is able to absorb some of the compression force applied to gastric residence system 502a, and more force is required to compress the first portion of the arms, preventing it from bending to the extent gastric residence system 502a bends in FIG. 5A.

FIG. 5C shows gastric residence system 502c having a first segment 504c and a second segment 506c. As shown, second segment 506c is smaller than second segment 506b of FIG. 5B. Thus, first segment 506c is larger than first segment 506b of FIG. 5B. Like gastric residence system 502b of FIG. 5B, second segment 504c absorbs some of the compression forces applied to gastric residence system 502c, preventing it from bending to the extent gastric residence system 502a bends in FIG. 5A. Additionally, gastric residence system 502c is compressed less than gastric residence system 502b of FIG. 5B.

The ratio of the first segment of a relatively flexible arm to the second segment of the arm may vary. If the first segment is too large in comparison to the second segment, the compression forces may transfer to the core of a gastric residence system too early, allowing the compression forces to compress the gastric residence system into a bended configuration small enough to prematurely pass through a pylorus. If the second segment is too large compared to the first segment, the second segment may too easily bend under the compression forces, allowing the forces to compress the gastric residence system into a bended configuration small enough to prematurely pass through a pylorus. Both scenarios result in a gastric residence system that is not as effective at resisting premature passage through the pylorus as desired.

An effective ratio of the first segment to the second segment of a flexible arm of a gastric residence system may vary. In some embodiments, the first segment may comprise from 10-90°/of a length of an arm (as measured from the proximal end to the distal end). In some embodiments, the first segment may comprise less than 90%, less than 80%, less than 70%, less than 60%, less than 50%, less than 40%, less than 30%, or less than 20% of a length of an arm. In some embodiments, the first segment may comprise more than 10%, more than 20%, more than 30%, more than 40%, more than 50%, more than 60%, more than 70%, or more than 80% of a length of an arm. In some embodiments, the second segment may comprise from 10-90% of a length of an arm (as measured from the proximal end to the distal end). In some embodiments, the second segment may comprise less than 90%, less than 80%, less than 70%, less than 60%, less than 50%, less than 40%, less than 30%, or less than 20% of a length of an arm. In some embodiments, the second segment may comprise more than 10%, more than 20%, more than 30%, more than 40%, more than 50%, more than 60%, more than 70%, or more than 80% of a length of an arm.

In some embodiments, one or more elements of a gastric residence system may be coated with a release-rate modulating polymer film. For example, one or more arms of a gastric residence system may be coated with a release-rate modulating polymer film. A release-rate modulating polymer film may help reduce an initial burst release of a therapeutic agent or API from the gastric residence system when administered to a patient. A release-rate modulating polymer film may help improve the overall linearity of the therapeutic agent/API release. A release-rate modulating polymer film may be applied to one or more elements of a gastric residence system using a pan coating process. In particular, the coating components were dissolved in ethyl acetate and pan coating was performed using a Freund-Vector LDCS Hi-Coater Lab Coater.

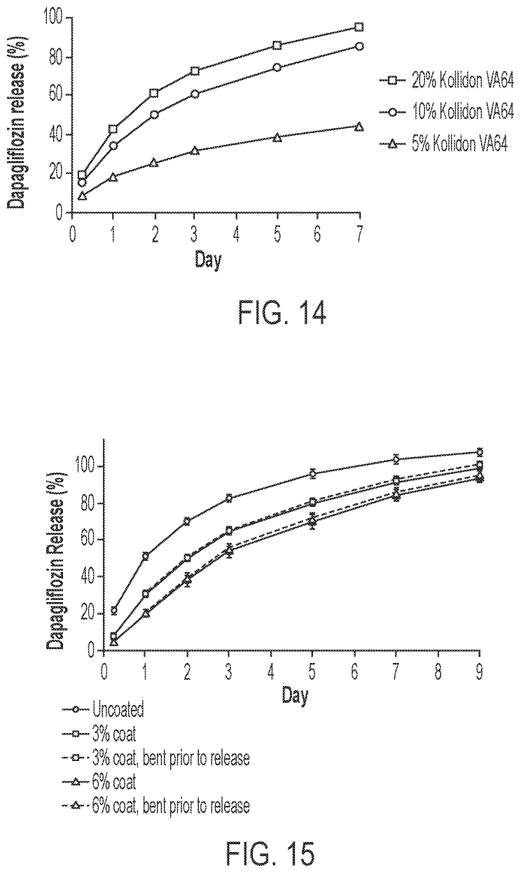

A greater coating weight can provide a thicker diffusion barrier for slower release. The release rate of the therapeutic agent/API may additionally be tuned by varying the coating porosity, which can be achieved by changing the content of a water-soluble excipient (e.g., copovidone) in the coating. A higher porosity may lead to a faster release. FIG. 14 shows data results of gastric residence systems having a release-rate modulating polymer film. These results are described in the “Examples” section.

In some embodiments, the release-rate modulating polymer film may comprise a polymer. For example, suitable polymers may include polyesters that can be used in the invention include polyesters with aliphatic groups as their main chains, including polylactones such as polycaprolactone (PCL); polyglycolic acid (PGA); polylactic acid (PLA); poly(lactic-co-glycolic acid) (PLGA); polyhydroxyalkanoates (PHA) such as polyhydroxybutyrate (PHB), polyhydroxyvalerate (PHV), and poly(3-hydroxybutyrate-co-3-hydroxyvalerate) (PHBV); polyethylene adipate (PEA); polybutylene succinate (PBS); and polyesters with aromatic groups in their main chains, such as polyethylene terephthalate (PET), polybutylene terephthalate (PBT), polytrimethylene terephthalate (PTT), and polyethylene naphthalate (PEN). Heteropolymers, including block or random copolymers, such as block or random copolymers incorporating the monomer constituents of the above polyesters, can also be used, including copolymers of lactide and caprolactone (poly-lactide-co-caprolactone; PLC). Mixtures of two or more polyesters can also be used. In addition to polyesters, cellulose acetate (CA), ethyl cellulose (EC), and copolymers of acrylate and methacrylate esters (e.g., Eudragit RS) can also be used as release rate-modulating polymer films. Release-rate modulating polymer films can comprise polyesters with a repeating unit of the form: —R1-O—C(═O)—, wherein R1 is selected from the group consisting of C1-C12 alkylene groups, such as C1-C8 alkylene groups or C1-C4 alkylene groups, ethers containing between two and twelve carbon atoms, two and eight carbon atoms or two and four carbon atoms, and polyethers containing between three and twelve carbon atoms or between three and eight carbon atoms. The polyesters can terminate with hydroxy groups, hydrogens, —C1-C12 alkyl groups, —C1-C8 alkyl groups, or —C1-C4 alkyl groups, or —C1-C12-OH, —C1-C8-OH, or —C1-C4-OH (alcohol) groups as appropriate. In some embomdiments, the R1 groups can be the same moiety throughout the polymer to form a homopolymer. In some embodiments, the R1 groups can be chosen from two or more different moieties, to form a heteropolymer. The heteropolymer can be a random copolymer, or a block copolymer. The release-rate modulating polymer film can comprise at least two different polyesters, each different polyester with a repeating unit of the form: —Rn—O—C(═O)—, wherein when at least two or more of the different polyesters are homopolymers, the Rn group of any one of the homopolymers is different from the Rn group of any other of the homopolymers; and when at least two or more of the different polyesters are heteropolymers, each heteropolymer has a different varying pattern of Rn groups than the varying pattern of Rn groups of any of the other heteropolymers; and each Rn is selected from the group consisting of C1-C12 alkylene groups, ethers containing between two and twelve carbon atoms, and polyethers containing between three and twelve carbon atoms.

In some embodiments, the release-rate modulating polymer film may comprise 30-90 wt. % polymer. In some embodiments, the release-rate modulating polymer film may comprise less than 90 wt. %, less than 80 wt. %, less than 70 wt. %, less than 60 wt. %, less than 50 wt. %, or less than 40 wt. % polymer. In some embodiments, the release-rate modulating polymer film may comprise more than 30 wt. %, more than 40 wt. %, more than 50 wt. %, more than 60 wt. %, more than 70 wt. %, or more than 80 wt. % polymer.

In some embodiments, the release-rate modulating polymer film may comprise one or more excipients. For example, suitable excipients may include porogens, plasticizers, or both porogens and plasticizers to further tune the release rate of the agent in the carrier polymer-agent segment.

Porogens are soluble additives that dissolve out of the release rate-modulating polymer films, creating pores in the films. In some embodiments, the porogens dissolve out of the films when the gastric residence systems are deployed in the gastric environment. That is, after preparation of the segments, the porogens are left in the segments which are assembled into the gastric residence system, and in the gastric residence system as administered to a patient; the porogens then dissolve out of the release rate-modulating polymer film when the gastric residence system is administered to the patient and contacts the gastric environment. In another embodiment, the porogens are removed from the film-covered carrier polymer-agent segments before the segments are assembled into the gastric residence system, or the porogens are removed from the gastric residence system before deployment of the gastric residence system in the gastric environment.

Porogens can be organic or inorganic materials. Examples of porogens include alkali metal salts such as sodium chloride, sodium bromide, potassium chloride, potassium sulfate, potassium phosphate, sodium benzoate, sodium acetate, sodium citrate, potassium nitrate and the like; alkaline earth metal salts such as calcium chloride, calcium nitrate, and the like; and transition metal salts such as ferric chloride, ferrous sulfate, zinc sulfate, cupric chloride, and the like. Additional examples of porogens include saccharides and sugars, such as sucrose, glucose, fructose, mannose, galactose, aldohexose, altrose, talose, lactose, cellulose, monosaccharides, disaccharides, and water soluble polysaccharides. Additional examples of porogens include sorbitol, mannitol, organic aliphatic and aromatic oils, including diols and polyols, as exemplified by polyhydric alcohols, poly(alkylene glycols), polyglycols, alkylene glycols, poly(a,m)alkylenediol esters or alkylene glycols, poly vinylalcohol, poly vinyl pyrrolidone, and water soluble polymeric materials. Further examples of porogens that can be used include Poloxamer; hypromellose (HPMC); Kolliphor RH40; polyvinyl caprolactam; polyvinyl acetate (PVAc); polyethylene glycol (PEG); Soluplus (available from BASF; a copolymer of polyvinyl caprolactam, polyvinyl acetate, and polyethylene glycol); copovidone; Eudragits (E, RS, RL); poly(methyl vinyl ether-alt-maleic anhydride); polyoxyethylene alkyl ethers; polysorbates; polyoxyethylene stearates; polydextrose; polyacrylic acid; alginates; sodium starch glycolate (SSG); crosslinked polyacrylic acid (carbopol); crosslinked PVP (crospovidone); crosslinked cellulose (croscarmellose); calcium silicate; xanthan gum; and gellan gum. Some particularly useful porogens include povidone, copovidone, and polyoxyl castor oil.

Porogens can be added to make up between about 1% to about 30% by weight of the release rate-modulating polymer film. Porogens can be added to make up about 1% to about 25%, about 1% to about 20%, about 1% to about 15%, about 1% to about 10%, about 1% to about 8%, about 1% to about 5%, about 1% to about 3%, about 5% to about 30%, about 10% to about 30%, about 15% to about 30%, about 20% to about 30%, or about 25% to about 30% by weight of the release rate-modulating polymer film. A preferred range of porogen is about 5% to about 20%, more preferably about 10% to about 20%, by weight of the release rate-modulating polymer film.

Plasticizers can also be added to further tune the properties of the release rate-modulating polymer films. Plasticizers that can be used include the classes of phthalates, phosphates, citrates, tartrates, adipates, sebacates, sulfonamides, succinates, glycolates, glycerolates, benzoates, myristates, and halogenated phenyls. Specific plasticizers that can be used include triacetin, triethyl citrate, PEG, poloxamer, tributyl citrate, and dibutyl sebacate. Triacetin and triethyl citrate (TEC) are particularly useful.

Plasticizers can be added to make up about 1% to about 35%, about 1% to about 30%, about 1% to about 25%, about 1% to about 20%, about 1% to about 15%, about 1% to about 10%, about 1% to about 8%, about 1% to about 5%, about 1% to about 3%, about 5% to about 40%, about 10% to about 40%, about 15% to about 40%, about 20% to about 40%, about 25% to about 40%, about 30% to about 40%, about 10% to about 30%, about 15% to about 30%, about 20% to about 30%, about 25% to about 30%, or about 10%, about 15%, about 20%, about 25%, about 30%, about 35%, or about 40% by weight of the release rate-modulating polymer film. A preferred range of plasticizer is about 5% to about 20%, more preferably about 10% to about 20%, by weight of the release rate-modulating polymer film.

Processing aids can also be added to release rate-modulating polymer films. Anti-tack agents, such as magnesium stearate, talc, or glycerol monostearate can be added to aid in processing of the films. Such anti-tack agents can be added in amounts of about 0.5% to about 5%, about 1% to about 3%, or about 2%.

The release-rate modulating polymer film can further comprise a permeable component which is permeable to the agent or pharmaceutically acceptable salt thereof, permeable to water, or permeable both to the agent or salt thereof and to water. Permeability components can thus function to increase the rate of water influx into the carrier polymer of the gastric residence system, and increase the rate of release of agent or salt thereof out of the gastric residence system. The permeable component can be a polymer or a swellable material. The permeable component can comprise about 1% to about 30% by weight of the film. The permeable component can be selected from the group consisting of SSG (sodium starch glycolate), crospovidone, croscarmellose, and Carbopol (PAA; crosslinked polyacrylic acid). At least one of the rate of passage of water and the rate of passage of agent or salt thereof through the permeable component should be higher, as compared to the rate of passage of water or the rate of passage of agent through the release-rate modulating polymer film lacking permeable agent and lacking pores produced by removal of porogens. In various embodiments, the rate of passage of water, the rate of passage of agent or salt thereof, or both the rate of passage of water and the rate of passage of agent or salt thereof through the permeable component is up to about 1.5 times, up to about 2 times, up to about 3 times, up to about 4 times, up to about 5 times, up to about 6 times, up to about 7 times, up to about 8 times, about to about 10 times, up to about 15 times, up to about 20 times, up to about 25 times, up to about 50 times, or up to about 100 times faster, as compared to the rate of passage of water or the rate of passage of agent or salt thereof, or both the rate of passage of water and the rate of passage of agent or salt thereof through the release-rate modulating polymer film lacking permeable agent and lacking pores produced by removal of porogens.

In some embodiments, the release-rate modulating polymer film may comprise 10-70 wt. % excipients. In some embodiments, the release-rate modulating polymer film may comprise less than 70 wt. %, less than 60 wt. %, less than 50 wt. %, less than 40 wt. %, less than 30 wt. %, or less than 20 wt. % excipients. In some embodiments, the release-rate modulating polymer film may comprise more than 10 wt. %, more than 20 wt. %, more than 30 wt. %, more than 40 wt. %, more than 50 wt. %, or more than 60 wt. % excipients.

Carrier Polymer-Agent/Agent Salt Combinations with Excipients and Other Additives

The blend of carrier polymer-agent or carrier polymer-agent salt can comprise various excipients and other additives. The following Table CPE-1 lists combinations of excipients and other additives that can be used in combination with agent or salt thereof and carrier polymer in the compositions making up the arms or segments of arms of the gastric residence systems. These excipients and other additives can be combined with agent or salt thereof (where the agent or agent salt comprises between about 10% to about 60% by weight of the composition) with the carrier polymer, such as polycaprolactone, making up the remainder of the composition. Excipients include the following, which can be used individually or in any combination, in amounts ranging from about 1% to about 30%, such as about 5% to about 20%, by weight of the composition: Kolliphor P407 (poloxamer 407, poly(ethylene glycol)-block-poly(propylene glycol)-block-poly(ethylene glycol)), Eudragit RS (Poly[Ethyl acrylate, methyl methacrylate, trimethylammonioethyl methacrylate chloride] 1:2:0.1), Eudragit RL (Poly[Ethyl acrylate, methyl methacrylate, trimethylammonioethyl methacrylate chloride] 1:2:0.2), PDO (polydioxanone), PEG-PCL, SIF (FaSSIF/FaSSGF powder from BioRelevant), EPO (dimethylaminoethyl methacrylate-butyl methacrylate-methyl methacrylate copolymer), Kollidon VA64 (vinylpyrrolidone-vinyl acetate copolymer in a ratio of 6:4 by mass), polyvinyl acetate, polyvinyl pyrrolidine.

Other additives include silicon dioxide (comprising, for example, about 0.1% to about 5% by weight of the composition, such as about 0.1% to 1% or about 0.5%) and an anti-oxidant, such as alpha-tocopherol (comprising, for example, about 0.1% to about 5% by weight of the composition, such as about 0.1% to 1% or about 0.5%). Each row of the table below represents a formulation of excipients and other additives for use with the carrier polymer and agent or salt thereof.

TABLE CPE-1
Excipients and additives, in combination with
agent or salt thereof and carrier polymer

EPO, P407, Silica, α-tocopherol
EPO, Silica, α-tocopherol
Eudragit RL, Eudragit RS, Kolliphor P407, Silica, α-tocopherol
Eudragit RL, Kolliphor P407, Silica, α-tocopherol
Eudragit RL, Eudragit RS, Kolliphor P407, Silica, α-tocopherol
Eudragit RL, Kolliphor P407, Silica, α-tocopherol
Eudragit RL, Kolliphor P407, Silica, α-tocopherol
Eudragit RS, P407, Silica, α-tocopherol
Eudragit RS, Silica, α-tocopherol
Kollidon VA64, Silica, α-tocopherol
Kolliphor P407, Silica, α-tocopherol
Kolliphor RH40, Silica, α-tocopherol
PDO, Silica, α-tocopherol
PEG-PCL, Silica, α-tocopherol
Poly Vinyl Acetate, Silica, α-tocopherol
PVP, Silica, α-tocopherol
SIF, Silica, α-tocopherol
Silica, P188, P407, α-tocopherol
Silica, α-tocopherol

Table CPE-2 lists specific amounts of excipients and other additives that can be used in combination with agent or salt thereof and carrier polymer in the compositions making up the arms or segments of arms of the gastric residence systems.

The amounts listed in Table CPE-2 can be varied by plus-or-minus 20% of each ingredient (for example, 0.5% silica can vary between 0.4% and 0.6% silica, as 20% of 0.5% is 0.1%). Each row of the table below represents a formulation of excipients and other additives for use with the carrier polymer and agent or salt thereof.

TABLE CPE-2
Excipients and additives, in combination with
agent or salt thereof and carrier polymer

0.5% Silica, 0.5% α-tocopherol
0.5% Silica, 2% P407, 0.5% α-tocopherol
0.5% Silica, 2% P188, 2% P407, 0.5% α-tocopherol
0.5% Silica, 3% Eudragit RS, 2% P407, 0.5% α-tocopherol
1% Kolliphor P407, 0.5% Silica, 0.5% α-tocopherol
10% Eudragit RS, 2.5% P407, 2% Silica, 0.5% α-tocopherol
10% Eudragit RS, 5% P407, 0.5% Silica, 0.5% α-tocopherol
10% Eudragit RS, 5% P407, 2% Silica, 0.5% α-tocopherol
12% Eudragit RL, 3% Kolliphor P407, 0.5% Silica, 0.5% α-tocopherol
12% Eudragit RL, 5% Kolliphor P407, 0.5% Silica, 0.5% α-tocopherol
14.78% Eudragit RS, 0.226% P407, 0.5% Silica, 0.5% α-tocopherol
17.5% Eudragit RS, 5% P407, 0.5% Silica, 0.5% α-tocopherol
19.8% Eudragit RS, 0.5% Silica, 0.5% α-tocopherol
2% Kolliphor P407, 0.5% Silica, 0.5% α-tocopherol
2% P407, 0.5% Silica, 0.5% α-tocopherol
20% Eudragit RS, 2% P407, 0.5% Silica, 0.5% α-tocopherol
21.25% Eudragit RS, 2.5% P407, 0.5% Silica, 0.5% α-tocopherol
25% Eudragit RL, 5% P407, 0.5% Silica, 0.5% α-tocopherol
25% Eudragit RS, 0.5% Silica, 0.5% α-tocopherol
25% Eudragit RS, 5% P407, 0.5% Silica, 0.5% α-tocopherol
3% Eudragit RL, 9% Eudragit RS, 5% Kolliphor P407, 0.5% Silica, 0.5%
α-tocopherol
3.5% Eudragit RS, 2.5% P407, 2% Silica, 0.5% α-tocopherol
3.5% Eudragit RS, 5% P407, 2% Silica, 0.5% α-tocopherol
30% PDO, 0.5% Silica, 0.5% α-tocopherol
39.5% PEG-PCL, 0.36% Silica, 0.36% α-tocopherol
4.5% EPO, 4.5% P407, 0.5% Silica, 0.5% α-tocopherol
5% Kolliphor P407, 0.5% Silica, 0.5% α-tocopherol
5% Kolliphor RH40, 0.5% Silica, 0.5% α-tocopherol
5% SIF, 0.5% Silica, 0.5% α-tocopherol
6% Eudragit RL, 5% Kolliphor P407, 0.5% Silica, 0.5% α-tocopherol
6% Eudragit RL, 6% Eudragit RS, 5% Kolliphor P407, 0.5% Silica, 0.5%
α-tocopherol
6.75% Eudragit RS, 3.75% P407, 2% Silica, 0.5% α-tocopherol
7% EPO, 2% P407, 0.5% Silica, 0.5% α-tocopherol
9% EPO, 0.5% Silica, 0.5% α-tocopherol
9% Eudragit RL, 3% Eudragit RS, 5% Kolliphor P407, 0.5% Silica, 0.5%
α-tocopherol
9% Kollidon VA64, 0.5% Silica, 0.5% α-tocopherol
9% Poly Vinyl Acetate, 0.5% Silica, 0.5% α-tocopherol
9% PVP, 0.5% Silica, 0.5% α-tocopherol
9% SIF, 0.5% Silica, 0.5% α-tocopherol

Exemplary formulations of a carrier polymer-agent arm segment for use in the invention are provided in the table below (provided as approximate weight percentages, with the understanding that the sum of all components equals 100%). These formulations can be used with any of the agents disclosed herein, such as dapagliflozin.

| Component | Formulation 1 | Formulation 2 | Formulation 3 |
|---|---|---|---|
| Agent | 10-30 | 15-25 | 20 |
| TPU | 40-60 | 45-55 | 49 |
| Copovidone | 10-30 | 15-25 | 20 |
| Poly-D,L-lactide | 5-15 | 7.5-12.5 | 10 |
| Vitamin E succinate | 0.1-2 | 0.3-0.7 | 0.5 |
| Colloidal SiO₂ | 0.1-2 | 0.3-0.7 | 0.5 |

### Agents for Use in Gastric Residence Systems

Agents (e.g., active pharmaceutical ingredient, therapeutic agent) which can be administered to or via the gastrointestinal tract can be used in the gastric residence systems of the invention. The agent is blended with the carrier polymer, and any other excipients or other additives to the carrier polymer, and formed into a segment for use in a gastric residence system. Agents include, but are not limited to, drugs, pro-drugs, biologics, and any other substance which can be administered to produce a beneficial effect on an illness or injury.

Agents that can be used in the gastric residence systems of the invention include statins, such as rosuvastatin; nonsteroidal anti-inflammatory drugs (NSAIDs) such as meloxicam; selective serotonin reuptake inhibitors (SSRIs) such as escitalopram and citalopram; blood thinners, such as clopidogrel; steroids, such as prednisone; antipsychotics, such as aripiprazole and risperidone; analgesics, such as buprenorphine; opioid antagonists, such as naloxone; anti-asthmatics such as montelukast; anti-dementia drugs, such as memantine; cardiac glycosides such as digoxin; alpha blockers such as tamsulosin; cholesterol absorption inhibitors such as ezetimibe; anti-gout treatments, such as colchicine; antihistamines, such as loratadine and cetirizine, opioids, such as loperamide; proton-pump inhibitors, such as omeprazole; antiviral agents, such as entecavir; antibiotics, such as doxycycline, ciprofloxacin, and azithromycin; anti-malarial agents; levothyroxine; substance abuse treatments, such as methadone and varenicline; contraceptives; stimulants, such as caffeine; and nutrients such as folic acid, calcium, iodine, iron, zinc, thiamine, niacin, vitamin C, vitamin D, biotin, plant extracts, phytohormones, and other vitamins or minerals. Biologics that can be used as agents in the gastric residence systems of the invention include proteins, polypeptides, polynucleotides, and hormones. Exemplary classes of agents include, but are not limited to, analgesics; anti-analgesics; anti-inflammatory drugs; antipyretics; antidepressants; antiepileptics; antipsychotic agents; neuroprotective agents; anti-proliferatives, such as anti-cancer agents; antihistamines; antimigraine drugs; hormones; prostaglandins; antimicrobials, such as antibiotics, antifungals, antivirals, and antiparasitics; anti-muscarinics; anxiolytics; bacteriostatics; immunosuppressant agents; sedatives; hypnotics; antipsychotics; bronchodilators; anti-asthma drugs; cardiovascular drugs; anesthetics; anti-coagulants; enzyme inhibitors; steroidal agents; steroidal or non-steroidal anti-inflammatory agents; corticosteroids; dopaminergics; electrolytes; gastro-intestinal drugs; muscle relaxants; nutritional agents; vitamins; parasympathomimetics; stimulants; anorectics; anti-narcoleptics; and antimalarial drugs, such as quinine, lumefantrine, chloroquine, amodiaquine, pyrimethamine, proguanil, chlorproguanil-dapsone, sulfonamides (such as sulfadoxine and sulfamethoxypyridazine), mefloquine, atovaquone, primaquine, halofantrine, doxycycline, clindamycin, artemisinin, and artemisinin derivatives (such as artemether, dihydroartemisinin, arteether and artesunate). The term “agent” includes salts, solvates, polymorphs, and co-crystals of the aforementioned substances. In some embodiments, the agent is selected from the group consisting of cetirizine, rosuvastatin, escitalopram, citalopram, risperidone, olanzapine, donepezil, and ivermectin. In some embodiments, the agent is one that is used to treat a neuropsychiatric disorder, such as an anti-psychotic agent or an anti-dementia drug such as memantine.

In some embodiments, the agent can exclude adamantane-class drugs. In some embodiments, the agent can exclude any one or more of memantine; amantadine; adapromine; nitromemantine; rimantadine; bromantane; neramexane; or tromantadine; or a pharmaceutically acceptable salt of memantine, amantadine, adapromine, nitromemantine, rimantadine, bromantane, or tromantadine. In some embodiments, the agent can exclude memantine. In some embodiments, the agent can exclude a salt of memantine or a pharmaceutically acceptable salt of memantine.

Agents can be used in the gastric residence systems of the invention in any suitable crystalline form, or in amorphous form, or in both crystalline form or forms and amorphous forms. That is, agent or drug particles contained in the gastric residence systems can be used in crystalline form, in amorphous form, or in a mixture of crystalline forms (either a single crystalline form, or multiple crystalline forms) and amorphous forms, so as to provide a desired rate of release or desired physical or chemical properties.

Gastric residence systems are well-suited for use in treatment of diseases and disorders which present difficulties with patient compliance, and thus in some embodiments, the gastric residence systems are used to treat a disease or disorder where patient compliance with a medication regimen is problematic. Such diseases and disorders include neuropsychiatric diseases and disorders, dementia and other diseases and disorders which affect memory, Alzheimer's disease, psychoses, schizophrenia, and paranoia. Accordingly, agents which can be used in the gastric residence systems include, but are not limited to, anti-dementia agents, anti-Alzheimer's disease agents, and anti-psychotics.

Exemplary hydrophilic agents which can be used in the systems include risperidone, cetirizine, memantine, and olanzapine. Exemplary hydrophobic agents which can be used in the systems include aripiprazole, ivermectin, rosuvastatin, citalopram, and escitalopram.

In some embodiments, the agent or salt thereof (for example, a drug) makes up about 10% to about 40% by weight of the arm or segment, and thus the carrier polymer and any other components of the arm or segment blended into the carrier polymer together make up the remainder of the weight of the arm or segment. In some embodiments, the agent or salt thereof makes up about 10% to about 35%, about 10% to about 30%, about 10% to about 25%, about 10% to about 20%, about 10% to about 15%, about 15% to about 40%, about 20% to about 40%, about 25% to about 40%, about 30% to about 40%, about 35% to about 40%, about 15% to about 35%, about 20% to about 35%, or about 25% to about 40% by weight of the arm or segment.

Further embodiments of arms or segments, where the agent or salt thereof makes up more than about 40% by weight of the arm or segment, are described below under “high agent loading of arms and segments.”

### High Agent Loading of Arms and Segments

In some embodiments of the invention, the arms, or segments of which the arms are comprised, can have a high loading of agent or pharmaceutically acceptable salt thereof. “High loading” generally refers to arms or segments where the agent or salt thereof (for example, a drug) makes up more than about 40% by weight of the arm or segment, and thus the carrier polymer and any other components of the arm or segment blended into the carrier polymer together make up less than about 60% by weight of the arm or segment. Any components of the arms or segments which are not blended into the carrier polymer are not included in the calculation of the weight percentage; for example, if an arm has one or more disintegrating matrices interspersed between segments of the arm, the weight of such matrices would not be included as part of the weight of the arm in the calculation of the weight percentage of agent in the arm. Once the loading of the agent increases to about 60%, it becomes increasingly difficult to properly blend the agent with the carrier polymer, and phase separation of the agent and polymer tends to occur. Thus, the loading of the agent in an arm or segment should not exceed about 60% of the total weight of the arm.

Thus, in some embodiments, the amount of agent by weight in the arms, or segments of which the arms are comprised, can comprise at least about 40%, at least about 45%, at least about 50%, at least about 55%, or about 60%. In some embodiments, the amount of agent by weight in the arms, or segments of which the arms are comprised, can comprise about 40% to about 60%, about 45% to about 60%, about 50% to about 60%, about 55% to about 60%, about 40% to about 55%, about 40% to about 50%, or about 40% to about 45%. In some embodiments, the amount of agent by weight in the arms, or segments of which the arms are comprised, can comprise about 25% to about 60%, about 30% to about 60%, or about 35% to about 60%. In some embodiments, the amount of agent by weight in the arms, or segments of which the arms are comprised, can comprise about 51% to about 60%, about 52% to about 60%, about 53% to about 60%, about 54% to about 60%, about 55% to about 60%, about 56% to about 60%, or about 57% to about 60%. In some embodiments, the agent or pharmaceutically acceptable salt thereof is present in an amount by weight of between about 67% and about 150% of the weight of the carrier polymer.

The combination of the high agent or agent salt loading with the release rate-controlling polymer film provides gastric residence systems with increased amounts of agent or agent salt, while maintaining good release kinetics over the residence period of the system.

### Dispersants for Modulation of Agent Release and Stability of Polymer Blend

The use of a dispersant in the carrier polymer-agent component provides numerous advantages. The rate of elution of agent from the carrier polymer-agent component is affected by numerous factors as previously noted, including the composition and properties of the carrier polymer (which may itself comprise multiple polymeric and non-polymeric components); the physical and chemical properties of the agent; and the gastric environment. Avoiding burst release of agent, especially hydrophilic agents, and maintaining sustained release of the agent over the effective release period or residence period is an important characteristic of the systems. The use of a dispersant according to the invention enables better control of release rate and suppression of burst release. Burst release and release rate can be tuned by using varied concentrations of dispersant. For example, different dispersants and different excipients, at varying concentrations, can tune burst release of cetirizine in simulated gastric fluid.

Dispersants which can be used in the invention include: silicon dioxide (silica, SiO2) (hydrophilic fumed); stearate salts, such as calcium stearate and magnesium stearate; microcrystalline cellulose; carboxymethylcellulose; hydrophobic colloidal silica; hypromellose; magnesium aluminum silicate; phospholipids; polyoxyethylene stearates; zinc acetate; alginic acid; lecithin; fatty acids; sodium lauryl sulfate; and non-toxic metal oxides such as aluminum oxide. Porous inorganic materials and polar inorganic materials can be used. Hydrophilic-fumed silicon dioxide is a preferred dispersant. One particularly useful silicon dioxide is sold by Cabot Corporation (Boston, Mass., USA) under the registered trademark CAB-O-SIL® M-5P (CAS #112945-52-5), which is hydrophilic-fumed silicon dioxide having a BET surface area of about 200 m2/g±15 m2/g The mesh residue for this product on a 45 micron sieve is less than about 0.02%. The typical primary aggregate size is about 150 to about 300 nm, while individual particle sizes may range from about 5 nm to about 50 nm.

In addition to anti-aggregation/anti-flocculation activity, the dispersant can help prevent phase separation during fabrication and/or storage of the systems. This is particularly useful for manufacture of the systems by hot melt extrusion.

The weight/weight ratio of dispersant to agent substance can be about 0.1% to about 5%, about 0.1% to about 4%, about 0.1% to about 3%, about 0.1% to about 2%, about 0.1% to about 1%, about 1% to about 5%, about 1% to about 4%, about 1% to about 3%, about 1% to about 2%, about 2% to about 4%, about 2% to about 3%, about 3% to about 4%, about 4% to about 5%, or about 0.1%, about 0.5%, about 1%, about 2%, about 3%, about 4% or about 5%.

Dispersants can comprise about 0.1% to about 4% of the carrier polymer-agent components, such as about 0.1% to about 3.5%, about 0.1% to about 3%, about 0.1% to about 2.5%, about 0.1% to about 2%, about 0.1% to about 1.5%, about 0.1% to about 1%, about 0.1% to about 0.5%, or about 0.2% to about 0.8%.

Dispersants can also be used to modulate the amount of burst release of agent or pharmaceutically acceptable salt thereof during the initial period when the gastric residence system is administered. In embodiments of a gastric residence system that is to be administered once weekly, the burst release over the approximately first six hours after initial administration is less than about 8%, preferably less than about 6%, of the total amount of agent (or salt thereof) in the system. In embodiments of a gastric residence system that is to be administered once every three days, the burst release over the approximately first six hours after initial administration is less than about 12%, preferably less than about 10%, of the total amount of agent (or salt thereof) in the system. In embodiments of a gastric residence system that is to be administered once daily, the burst release over the approximately first six hours after initial administration is less than about 40%, preferably less than about 30%, of the total amount of agent (or salt thereof) in the system. In general, if a new gastric residence system is administered every D days, and the total mass of agent (or salt thereof) is M, then the gastric residence system releases less than about [(M divided by D) times 0.5], preferably less than about [(M divided by D) multiplied by 0.4], or less than about [(M divided by D) multiplied by 3/8], more preferably less than about [(M divided by D) multiplied by 0.3], over the approximately first six hours after initial administration. In further embodiments, the gastric residence system releases at least about [(M divided by D) multiplied by 0.25] over the approximately first six hours after initial administration, that is, the system releases at least about one-quarter of the daily dosage over the first one-quarter of the first day of administration.

### Stabilizers for Use in Gastric Residence Systems

Many agents are prone to oxidative degradation when exposed to reactive oxygen species, which can be present in the stomach. An agent contained in the system may thus oxidize due to the prolonged residence in the stomach of the system, and the extended release period of agent from the system. Accordingly, it is desirable to include stabilizers or preservatives in the systems, in order to stabilize the agent to prevent oxidative and other degradation.

Stabilizers, such as anti-oxidants including tocopherols, alpha-tocopherol, ascorbic acid, ascorbyl palmitate, butylated hydroxytoluene, butylated hydroxyanisole, and fumaric acid, can comprise about 0.1% to about 4% of the carrier polymer-agent components, such as about 0.1% to about 3.5%, about 0.1% to about 3%, about 0.1% to about 2.5%, about 0.1% to about 2%, about 0.1% to about 1.5%, about 0.1% to about 1%, about 0.1% to about 0.5%, or about 0.2% to about 0.8%.

Anti-oxidant stabilizers that can be included in the systems to reduce or prevent oxidation of the agent include alpha-tocopherol (about 0.01 to about 0.05% v/v), ascorbic acid (about 0.01 to about 0.1% w/v), ascorbyl palmitate (about 0.01 to about 0.1% w/v), butylated hydroxytoluene (about 0.01 to about 0.1% w/w), butylated hydroxyanisole (about 0.01 to about 0.1% w/w), and fumaric acid (up to 3600 ppm). Vitamin E, a tocopherol, a Vitamin E ester, a tocopherol ester, ascorbic acid, or a carotene, such as alpha-tocopherol, Vitamin E succinate, alpha-tocopherol succinate, Vitamin E acetate, alpha-tocopherol acetate, Vitamin E nicotinate, alpha-tocopherol nicotinate, Vitamin E linoleate, or alpha-tocopherol linoleate can be used as anti-oxidant stabilizers.

Certain agents can be pH-sensitive, especially at the low pH present in the gastric environment. Buffering or pH-stabilizer compounds that can be included in the systems to reduce or prevent degradation of agent at low pH include calcium carbonate, calcium lactate, calcium phosphate, sodium phosphate, and sodium bicarbonate. They are typically used in an amount of up to about 2% w/w. The buffering or pH-stabilizer compounds can comprise about 0.1% to about 4% of the carrier polymer-agent components, such as about 0.1% to about 3.5%, about 0.1% to about 3%, about 0.1% to about 2.5%, about 0.1% to about 2%, about 0.1% to about 1.5%, about 0.1% to about 1%, about 0.1% to about 0.5%, or about 0.2% to about 0.8%.

The anti-oxidant stabilizers, pH stabilizers, and other stabilizer compounds are blended into the polymers containing the agent (or pharmaceutically acceptable salt thereof) by blending the stabilizer(s) into the molten carrier polymer-agent or agent salt mixture. The stabilizer(s) can be blended into molten carrier polymer prior to blending the agent (or salt thereof) into the polymer-stabilizer mixture; or the stabilizer(s) can be blended with agent (or salt thereof) prior to formulation of the blended agent (or salt thereof)-stabilizer mixture in the carrier polymer; or stabilizer(s), agent (or salt thereof), and molten carrier polymer can be blended simultaneously. Agent (or salt thereof) can also be blended with molten carrier polymer prior to blending the stabilizer(s) into the polymer-agent or agent salt mixture.

In one embodiment, less than about 10% of the agent (or salt thereof) remaining in the system is degraded or oxidized after a gastric residence period of about 24 hours. In one embodiment, less than about 10% of the agent (or salt thereof) remaining in the system is degraded or oxidized after a gastric residence period of about 48 hours. In one embodiment, less than about 10% of the agent (or salt thereof) remaining in the system is degraded or oxidized after a gastric residence period of about 72 hours. In one embodiment, less than about 10% of the agent (or salt thereof) remaining in the system is degraded or oxidized after a gastric residence period of about 96 hours. In one embodiment, less than about 10% of the agent (or salt thereof) remaining in the system is degraded or oxidized after a gastric residence period of about five days. In some embodiments, less than about 10% of the agent (or salt thereof) remaining in the system is degraded or oxidized after a gastric residence period of about a week. In some embodiments, less than about 10% of the agent (or salt thereof) remaining in the system is degraded or oxidized after a gastric residence period of about two weeks.

In one embodiment, less than about 5% of the agent (or salt thereof) remaining in the system is degraded or oxidized after a gastric residence period of about 24 hours. In one embodiment, less than about 5% of the agent (or salt thereof) remaining in the system is degraded or oxidized after a gastric residence period of about 48 hours. In one embodiment, less than about 5% of the agent (or salt thereof) remaining in the system is degraded or oxidized after a gastric residence period of about 72 hours. In one embodiment, less than about 5% of the agent (or salt thereof) remaining in the system is degraded or oxidized after a gastric residence period of about 96 hours. In one embodiment, less than about 5% of the agent (or salt thereof) remaining in the system is degraded or oxidized after a gastric residence period of about five days. In some embodiments, less than about 5% of the agent (or salt thereof) remaining in the system is degraded or oxidized after a gastric residence period of about a week. In some embodiments, less than about 5% of the agent (or salt thereof) remaining in the system is degraded or oxidized after a gastric residence period of about two weeks.

### Coupling Polymers

The coupling polymer is used to link one or more carrier polymer-agent components (i.e., arm, or segment of an arm) to one or more carrier polymer-agent components, to link one or more carrier polymer-agent components to one or more elastomer components (i.e., core), or to link one or more elastomer components to one or more elastomer components. Thus, the coupling polymers form linker regions between other components of the system. Enteric polymers and time-dependent polymers are preferred for use as coupling polymers. In some embodiments, enteric polymers are used as coupling polymers. In some embodiments, time-dependent polymers which are pH-resistant, that is, less sensitive to changes in pH than enteric polymers, are used as coupling polymers. In some embodiments, both enteric polymers and time-dependent polymers which are less sensitive to changes in pH than enteric polymers are used as coupling polymers.

Enteric polymers are relatively insoluble under acidic conditions, such as the conditions encountered in the stomach, but are soluble under the less acidic to basic conditions encountered in the small intestine. Enteric polymers which dissolve at about pH 5 or above can be used as coupling polymers, as the pH of the initial segment of the small intestine, the duodenum, ranges from about 5.4 to 6.1. If the gastric residence system passes intact through the pyloric valve, the enteric coupling polymer will dissolve and the components linked by the coupling polymer will break apart, allowing passage of the residence system through the small and large intestines. Thus, the gastric residence systems are designed to uncouple rapidly in the intestinal environment by dissolution of the coupling polymer.

By “time-dependent polymer which are pH-resistant” (or equivalently, “pH-resistant time-dependent polymers”) is meant that, under conditions where an enteric polymer would degrade to the point that it would no longer link the components together, the time-dependent polymer will still have sufficient mechanical strength to link the components together. In some embodiments, the time-dependent polymer retains about the same linking capacity, that is, about 100% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 90% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 75% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 60% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 50% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 25% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer resists breaking under a flexural force of about 0.2 Newtons (N), about 0.3 N, about 0.4 N, about 0.5 N, about 0.75 N, about 1 N, about 1.5 N, about 2 N, about 2.5 N, about 3 N, about 4 N, or about 5 N, after exposure to a solution between about pH 7 to about pH 8, where the exposure is for about an hour, about a day, about three days, or about a week. Linkage strength can be measured by any relevant test that serves to test coupling ability, such as a four-point bending flexural test (ASTM D790).

Exemplary coupling polymers include, but are not limited to, cellulose acetate phthalate, cellulose acetate succinate, methylcellulose phthalate, ethylhydroxycellulose phthalate, polyvinylacetatephthalate, polyvinylbutyrate acetate, vinyl acetate-maleic anhydride copolymer, styrene-maleic mono-ester copolymer, methacrylic acid methylmethacrylate copolymer, methyl acrylate-methacrylic acid copolymer, methacrylate-methacrylic acid-octyl acrylate copolymer, and copolymers, mixtures, blends and combinations thereof. Some of the enteric polymers that can be used in the invention are listed in the Enteric Polymer Table, along with their dissolution pH. (See Mukherji, Gour and Clive G. Wilson, “Enteric Coating for Colonic Delivery,” Chapter 18 of Modified-Release Drug Delivery Technology (editors Michael J. Rathbone, Jonathan Hadgraft, Michael S. Roberts), Drugs and the Pharmaceutical Sciences Volume 126, New York: Marcel Dekker, 2002.) Preferably, enteric polymers that dissolve at a pH of no greater than about 5 or about 5.5 are used. Poly(methacrylic acid-co-ethyl acrylate) (sold under the trade name EUDRAGIT L 100-55; EUDRAGIT is a registered trademark of Evonik Rohm GmbH, Darmstadt, Germany) is a preferred enteric polymer. Another preferred enteric polymer is hydroxypropylmethylcellulose acetate succinate (hypromellose acetate succinate or HPMCAS; Ashland, Inc., Covington, Kentucky, USA), which has a tunable pH cutoff from about 5.5 to about 7.0. Cellulose acetate phthalate, cellulose acetate succinate, and hydroxypropyl methylcellulose phthalate are also suitable enteric polymers.

In one embodiment, the enteric polymers used in the gastric residence system dissolve at a pH above about 4. In some embodiments, the enteric polymers used in the gastric residence system dissolve at a pH above about 5. In some embodiments, the enteric polymers used in the gastric residence system dissolve at a pH above about 6. In some embodiments, the enteric polymers used in the gastric residence system dissolve at a pH above about 7. In some embodiments, the enteric polymers used in the gastric residence system dissolve at a pH above about 7.5. In some embodiments, the enteric polymers used in the gastric residence system dissolve at a pH between about 4 and about 5. In some embodiments, the enteric polymers used in the gastric residence system dissolve at a pH between about 4 and about 6. In some embodiments, the enteric polymers used in the gastric residence system dissolve at a pH between about 4 and about 7. In some embodiments, the enteric polymers used in the gastric residence system dissolve at a pH between about 4 and about 7.5. In some embodiments, the enteric polymers used in the gastric residence system dissolve at a pH between about 5 and about 6. In some embodiments, the enteric polymers used in the gastric residence system dissolve at a pH between about 5 and about 7. In some embodiments, the enteric polymers used in the gastric residence system dissolve at a pH between about 5 and about 7.5. In some embodiments, the enteric polymers used in the gastric residence system dissolve at a pH between about 6 and about 7. In some embodiments, the enteric polymers used in the gastric residence system dissolve at a pH between about 6 and about 7.5.

Enteric Polymer Table

| Polymer | Dissolution pH |
|---|---|
| Cellulose acetate phthalate | 6.0-6.4 |
| Hydroxypropyl methylcellulose phthalate 50 | 4.8 |
| Hydroxypropyl methylcellulose phthalate 55 | 5.2 |
| Polyvinylacetate phthalate | 5.0 |
| Methacrylic acid-methyl methacrylate copolymer (1:1) | 6.0 |
| Methacrylic acid-methyl methacrylate copolymer (2:1) | 6.5-7.5 |
| Methacrylic acid-ethyl acrylate copolymer (2:1) | 5.5 |
| Shellac | 7.0 |
| Hydroxypropyl methylcellulose acetate succinate | 7.0 |
| Poly (methyl vinyl ether/maleic acid) monoethyl ester | 4.5-5.0 |
| Poly (methyl vinyl ether/maleic acid) n-butyl ester | 5.4 |

Additional preferred polymers for use as coupling polymers are time-dependent polymers, that is, polymers that degrade in a time-dependent manner in the gastric environment. For example, the liquid plasticizer triacetin releases from a polymer formulation in a time-dependent manner over seven days in simulated gastric fluid, while Plastoid B retains its strength over a seven-day period in simulated gastric fluid. Thus, a polymer that degrades in a time-dependent manner can be readily prepared by mixing Plastoid B and triacetin; the degradation time of the Plastoid B-triacetin mixture can be extended by increasing the amount of Plastoid B used in the mixture (that is, using less triacetin in the mixture), while the degradation time can be decreased by decreasing the amount of Plastoid B used in the mixture (that is, using more triacetin in the mixture).

A variety of time-dependent mechanisms are available. Water-soluble time-dependent polymers break down as water penetrates through the polymer. Examples of such polymers are hydroxypropyl methylcellulose and poly vinyl acetate. Acid soluble time-dependent polymers break down over time in an acidic environment. Examples include Eudragit EPO. Time-dependent polymers can use water soluble plasticizers; as plasticizer is released, the remaining polymer becomes brittle and breaks under gastric forces. Examples of such polymers include triacetin and triethyl citrate.

In some embodiments, the carrier polymer-agent components are arms comprised of segments attached by enteric polymers. In some embodiments, the carrier polymer-agent components are attached to the elastomer component of the system by enteric polymers. In any of these embodiments, when enteric polymers are used for both segment-to-segment attachments and for attachment of the arms to the elastomeric component, the enteric polymer used for segment-segment attachments can be the same enteric polymer as the enteric polymer used for attachment of the arms to the elastomeric component, or the enteric polymer used for segment-segment attachments can be a different enteric polymer than the enteric polymer used for attachment of the arms to the elastomeric component. The enteric polymers used for the segment-segment attachments can all be the same enteric polymer, or can all be different enteric polymers, or some enteric polymers in the segment-segment attachments can be the same and some enteric polymers in the segment-segment attachments can be different. That is, the enteric polymer(s) used for each segment-segment attachment and the enteric polymer used for attachment of the arms to the elastomeric component can be independently chosen.

In some embodiments, the carrier polymer-drug components are non-segmented arms attached to the elastomer component of the system by enteric polymers, time-dependent linkers, or disintegrating matrices, or by any combination of enteric polymers, time-dependent linkers, and/or disintegrating matrices.

In any of the embodiments of the gastric residence systems described herein, the coupling polymers or linkers can comprise hydroxypropyl methyl cellulose acetate succinate (HPMCAS) and polycaprolactone (PCL). These blends can be used to form disintegrating linkers or disintegrating matrices. The ratio of HPMCAS to polycaprolactone in the disintegrating linker or disintegrating matrix can be between about 80% HPMCAS:20% PCL to about 20% HPMCAS:80% PCL. the ratio of HPMCAS to polycaprolactone can be between about 80% HPMCAS:20% PCL to about 20% HPMCAS:80% PCL; between about 70% HPMCAS:30% PCL to about 30% HPMCAS:70% PCL; between about 60% HPMCAS:40% PCL to about 40% HPMCAS:60% PCL; between about 80% HPMCAS:20% PCL to about 50% HPMCAS:50% PCL; between about 80% HPMCAS:20% PCL to about 60% HPMCAS:40% PCL; between about 70% HPMCAS:30% PCL to about 50% HPMCAS:50% PCL; between about 70% HPMCAS:30% PCL to about 60% HPMCAS:40% PCL; between about 20% HPMCAS:80% PCL to about 40% HPMCAS:60% PCL; between about 20% HPMCAS:80% PCL to about 50% HPMCAS:50% PCL; between about 30% HPMCAS:70% PCL to about 40% HPMCAS:60% PCL; between about 30% HPMCAS:70% PCL to about 50% HPMCAS:50% PCL; or about 80% HPMCAS:20% PCL, about 70% HPMCAS:30% PCL, about 60% HPMCAS:40% PCL, about 50% HPMCAS:50% PCL, about 40% HPMCAS:60% PCL, about 30% HPMCAS:70% PCL, or about 20% HPMCAS:80% PCL. The linker can further comprise a plasticizer selected from the group consisting of triacetin, triethyl citrate, tributyl citrate, poloxamers, polyethylene glycol, polypropylene glycol, diethyl phthalate, dibutyl sebacate, glycerin, castor oil, acetyl triethyl citrate, acetyl tributyl citrate, polyethylene glycol monomethyl ether, sorbitol, sorbitan, a sorbitol-sorbitan mixture, and diacetylated monoglycerides.

The linkers are chosen to weaken sufficiently after a specified period of time in order to allow the gastric residence systems to reach a point where they de-couple and pass through the pylorus and out of the stomach after the desired residence period or weaken sufficiently such that the gastric residence system is no longer retained in the stomach; that is, the linkers weaken to the point of uncoupling (the uncoupling point) or to the point where the gastric residence system can pass through the pylorus (the pyloric passage point, or passage point). Thus, in one embodiment, linkers are used that uncouple after about two days in a human stomach; after about three days in a human stomach; after about four days in a human stomach; after about five days in a human stomach; after about six days in a human stomach; after about seven days in a human stomach; after about eight days in a human stomach; after about nine days in a human stomach; after about ten days in a human stomach; or after about two weeks in a human stomach. In one embodiment, linkers are used that uncouple after about two days in a dog stomach; after about three days in a dog stomach; after about four days in a dog stomach; after about five days in a dog stomach; after about six days in a dog stomach; after about seven days in a dog stomach; after about eight days in a dog stomach; after about nine days in a dog stomach; after about ten days in a dog stomach; or after about two weeks in a dog stomach. In one embodiment, linkers are used that uncouple after about two days in a pig stomach; after about three days in a pig stomach; after about four days in a pig stomach; after about five days in a pig stomach; after about six days in a pig stomach; after about seven days in a pig stomach; after about eight days in a pig stomach; after about nine days in a pig stomach; after about ten days in a pig stomach; or after about two weeks in a pig stomach. In one embodiment, linkers are used that uncouple after about two days in fasted-state simulated gastric fluid; after about three days in fasted-state simulated gastric fluid; after about four days in fasted-state simulated gastric fluid; after about five days in fasted-state simulated gastric fluid; after about six days in fasted-state simulated gastric fluid; after about seven days in fasted-state simulated gastric fluid; after about eight days in fasted-state simulated gastric fluid; after about nine days in fasted-state simulated gastric fluid; after about ten days in fasted-state simulated gastric fluid; or after about two weeks in fasted-state simulated gastric fluid. In one embodiment, linkers are used that uncouple after about two days in fed-state simulated gastric fluid; after about three days in fed-state simulated gastric fluid; after about four days in fed-state simulated gastric fluid; after about five days in fed-state simulated gastric fluid; after about six days in fed-state simulated gastric fluid; after about seven days in fed-state simulated gastric fluid; after about eight days in fed-state simulated gastric fluid; after about nine days in fed-state simulated gastric fluid; after about ten days in fed-state simulated gastric fluid; or after about two weeks in fed-state simulated gastric fluid. In one embodiment, linkers are used that uncouple after about two days in water at pH 2; after about three days in water at pH 2; after about four days in water at pH 2; after about five days in water at pH 2; after about six days in water at pH 2; after about seven days in water at pH 2; after about eight days in water at pH 2; after about nine days in water at pH 2; after about ten days in water at pH 2; or after about two weeks in water at pH 2. In one embodiment, linkers are used that uncouple after about two days in water at pH 1; after about three days in water at pH 1; after about four days in water at pH 1; after about five days in water at pH 1; after about six days in water at pH 1; after about seven days in water at pH 1; after about eight days in water at pH 1; after about nine days in water at pH 1; after about ten days in water at pH 1; or after about two weeks in water at pH 1.

The de-coupling or pyloric passage point in human, dog, or pig occurs when the system passes out of the stomach, that is, when it passes through the pylorus. For the in vitro measurements in simulated gastric fluid or acidic water, the de-coupling or pyloric passage point occurs when the linker weakens to the point where it will break under the normal compressive forces of the stomach, typically about 0.1 Newton to 0.2 Newton. Linkage strength (breaking point) can be measured by any relevant test that serves to test coupling ability, that is, the force required to break the linker, such as the four-point bending flexural test (ASTM D790) described in Example 18 of WO 2017/070612, or Examples 12, 13, 15, 17, or 18 of WO 2017/100367. In one embodiment, the de-coupling or pyloric passage point is reached when the linkers uncouple at about 0.2 N of force. In another embodiment, the de-coupling or pyloric passage point is reached when the linkers uncouple at about 0.1 N of force.

The gastric residence systems can reach the pyloric passage point without any or all of the linkers actually breaking. If the linkers weaken or degrade to the point where they can no longer hold the gastric residence system in the stomach, even if one, some, or all of the linkers do not break, the gastric residence system will pass through the pylorus and into the small intestine (the pyloric passage point or passage point). In some embodiments, linkers are used that weaken to the passage point after about two days in a human stomach; after about three days in a human stomach; after about four days in a human stomach; after about five days in a human stomach; after about six days in a human stomach; after about seven days in a human stomach; after about eight days in a human stomach; after about nine days in a human stomach; after about ten days in a human stomach; or after about two weeks in a human stomach. In some embodiments, linkers are used that weaken to the passage point after about two days in a dog stomach; after about three days in a dog stomach; after about four days in a dog stomach; after about five days in a dog stomach; after about six days in a dog stomach; after about seven days in a dog stomach; after about eight days in a dog stomach; after about nine days in a dog stomach; after about ten days in a dog stomach; or after about two weeks in a dog stomach. In some embodiments, linkers are used that weaken to the passage point after about two days in a pig stomach; after about three days in a pig stomach; after about four days in a pig stomach; after about five days in a pig stomach; after about six days in a pig stomach; after about seven days in a pig stomach; after about eight days in a pig stomach; after about nine days in a pig stomach; after about ten days in a pig stomach; or after about two weeks in a pig stomach. In some embodiments, linkers are used that weaken to the passage point after about two days in fasted-state simulated gastric fluid; after about three days in fasted-state simulated gastric fluid; after about four days in fasted-state simulated gastric fluid; after about five days in fasted-state simulated gastric fluid; after about six days in fasted-state simulated gastric fluid; after about seven days in fasted-state simulated gastric fluid; after about eight days in fasted-state simulated gastric fluid; after about nine days in fasted-state simulated gastric fluid; after about ten days in fasted-state simulated gastric fluid; or after about two weeks in fasted-state simulated gastric fluid. In some embodiments, linkers are used that weaken to the passage point after about two days in fed-state simulated gastric fluid; after about three days in fed-state simulated gastric fluid; after about four days in fed-state simulated gastric fluid; after about five days in fed-state simulated gastric fluid; after about six days in fed-state simulated gastric fluid; after about seven days in fed-state simulated gastric fluid; after about eight days in fed-state simulated gastric fluid; after about nine days in fed-state simulated gastric fluid; after about ten days in fed-state simulated gastric fluid; or after about two weeks in fed-state simulated gastric fluid. In some embodiments, linkers are used that weaken to the passage point after about two days in water at pH 2; after about three days in water at pH 2; after about four days in water at pH 2; after about five days in water at pH 2; after about six days in water at pH 2; after about seven days in water at pH 2; after about eight days in water at pH 2; after about nine days in water at pH 2; after about ten days in water at pH 2; or after about two weeks in water at pH 2. In some embodiments, linkers are used that weaken to the passage point after about two days in water at pH 1; after about three days in water at pH 1; after about four days in water at pH 1; after about five days in water at pH 1; after about six days in water at pH 1; after about seven days in water at pH 1; after about eight days in water at pH 1; after about nine days in water at pH 1; after about ten days in water at pH 1; or after about two weeks in water at pH 1.

### Gastric Residence Arms Comprising a Filament

In some embodiments, gastric residence systems described herein may additionally comprise a filament to help prevent premature passage of the gastric residence system through a patient's pylorus.

Following is a description of gastric residence systems having a filament. As described, the filament of gastric residence systems having a filament may help prevent the gastric residence system from prematurely passing through a patient's pylorus.

A filament may be attached to the distal ends of the arms of a gastric residence system. In particular, a filament wrapped circumferentially around a gastric residence system connecting each arm can prevent one or two arms from prematurely entering the pylorus and pulling the rest of the system through, for example. It also maintains the spacing of the arms, which changes the bending geometry and increases the force required to compress the gastric residence system to a configuration small enough to prematurely pass through the pylorus. Filaments provided herein are flexible and stretchable such that they can maintain integrity despite gastric forces that may bend and contort gastric residence system. In some embodiments, a single filament may wrap circumferentially around a gastric residence system, connecting to each arm at the arm tip. In some embodiments, multiple filaments may connect each arm of gastric residence system 500b.

In some embodiments, a gastric residence system may comprise tips located at a distal end of one or more arms. The tips may comprise an enteric polymer composition. The filament may be connected to each arm by way of the tip at the distal end. The tips may be configured to separate from the rest of the arm when in a gastric environment. In particular, the tips may be configured to separate from the arms, allowing the filament to also separate from the gastric residence system. This separation may be fine-tuned such that the tips and filament separate once a predetermined gastric residence time approaches expiration, such that the gastric residence system separates and passes through a patient's pylorus at the expiration of the predetermined gastric residence time. If the tips and/or filament separate too early, the gastric residence system risks passing through the patient's pylorus prematurely.

In some embodiments, the arm tips may comprise one or more polymers, an enteric material, a plasticizer, and an acid. Suitable polymers may include polycaprolactone and/or thermoplastic polyurethanes (e.g. Pathway™ by Lubrizol). In some embodiments, the composition of an arm tip may be the same as the composition of a linker component. In some embodiments, the composition of an arm tip may be different than the composition of a linker component. In some embodiments, an arm tip may comprise from 10 to 50 wt. % polymer. In some embodiments, an arm tip may comprise less than 50 wt. %, less than 40 wt. %, less than 30 wt. %, or less than 20 wt. % polymer. In some embodiments, an arm tip may comprise more than 10 wt. %, more than 20 wt. %, more than 30 wt. %, or more than 40 wt. % polymer.

In some embodiments, the enteric material of the arm tips may comprise an enteric polymer. For example, suitable enteric polymers include Cellulose acetate phthalate, Hydroxypropyl methylcellulose phthalate 50, Hydroxypropyl methylcellulose phthalate 55, Polyvinylacetate phthalate, Methacrylic acid-methyl methacrylate copolymer (1:1), Methacrylic acid-methyl methacrylate copolymer (2:1), Methacrylic acid-ethyl acrylate copolymer (2:1), Shellac, Hydroxypropyl methylcellulose acetate succinate, Poly (methyl vinyl ether/maleic acid) monoethyl ester, or Poly (methyl vinyl ether/maleic acid)n-butyl ester. In some embodiments, an arm tip may comprise from 20 to 90 wt. % enteric material. In some embodiments, an arm tip may comprise less than 90 wt. %, less than 80 wt. %, less than 70 wt. %, less than 60 wt. %, less than 50 wt. %, less than 40 wt. %, or less than 30 wt. % enteric material. In some embodiments, an arm tip may comprise more than 20 wt. %, more than 30 wt. %, more than 40 wt. %, more than 50 wt. %, more than 60 wt. %, more than 70 wt. %, or more than 90 wt. % enteric material.

Suitable plasticizers may include propylene glycol, P407, triethyl citrate, triacetin, dibutyl sebacate, and/or polyethylene glycol. In some embodiments, an arm tip may comprise from 1 to 20 wt. % plasticizer. In some embodiments, an arm tip may comprise less than 20 wt. %, less than 15 wt. %, less than 10 wt. %, or less than 5 wt. % plasticizer. In some embodiments, an arm tip may comprise more than 1 wt. %, more than 5 wt. %, more than 10 wt. %, or more than 15 wt. % plasticizer.

Suitable acids can include stearic acid or other fatty acids. In some embodiments, an arm tip may comprise from 1 to 20 wt. % or from 1 to 10 wt. % acid. In some embodiments, an arm tip may comprise less than 20 wt. %, less than 15 wt. %, less than 10 wt. %, or less than 5 wt. % acid. In some embodiments, an arm tip may comprise more than 1 wt. %, more than 5 wt. %, more than 10 wt. %, or more than 15 wt. % acid.

Filaments for improved gastric residence may include elastic polymers and/or bioresorbable polymers.

Suitable elastic polymers may include Polyurethanes (Lubrizol Pellethane, Pathways, Tecoflex, carbothane), polyamide-polyether block copolymers (Pebax), poly(ethylene-co-vinyl acetate) (PEVAc), polyvinyl acetate, silicones, and/or combinations thereof. In some embodiments, a filament may comprise 10-90 wt. %, 20-80%, or 30-70 wt. % elastic polymer. In some embodiments, a filament may comprise less than 90 wt. %, less than 80 wt. %, less than 70 wt. %, less than 60 wt. %, less than 50 wt. %, less than 40 wt. %, less than 30 wt. %, or less than 20 wt. % elastic polymer. In some embodiments, a filament my comprise more than 10 wt. %, more than 20 wt. %, more than 30 wt. %, more than 40 wt. %, more than 50 wt. %, more than 60 wt. %, more than 70 wt. %, or more than 80 wt. % elastic polymer.

Suitable bioresorbable polymers can include Poly(lactic-co-glycolic acid) (PLGA), polycaprolactone (PCL), poly(lactic acid) (PLA), PCL-PLA copolymers, polydioxanone, poly(trimethylene carbonate), PCL-poly(glycolic acid) copolymers, Poly(glycerol sebacate), Polyanhydrides, Polyphosphazenes, Poly(alkyl cyanoacrylate)s, poly(amino acids), Poly(propylene fumarate), and/or combinations thereof. In some embodiments, a filament may comprise 10-90 wt. %, 20-80%, or 30-70 wt. % bioresorbable polymer. In some embodiments, a filament may comprise less than 90 wt. %, less than 80 wt. %, less than 70 wt. %, less than 60 wt. %, less than 50 wt. %, less than 40 wt. %, less than 30 wt. %, or less than 20 wt. % bioresorbable polymer. In some embodiments, a filament my comprise more than 10 wt. %, more than 20 wt. %, more than 30 wt. %, more than 40 wt. %, more than 50 wt. %, more than 60 wt. %, more than 70 wt. %, or more than 80 wt. % bioresorbable polymer.

In some embodiments, a filament may include a plasticizer. For example, suitable plasticizers can include propylene glycol, P407, triethyl citrate, triacetin, dibutyl sebacate, and/or polyethylene glycol. In some embodiments, a filament may include 0.1 to 20 wt. % plasticizer or 1 to 10 wt. % plasticizer. In some embodiments, a filament may comprise less than 20 wt. %, less than 15 wt. %, less than 10 wt. %, less than 5 wt. %, or less than 1 wt. % plasticizer. In some embodiments, a filament may comprise more than 0.1 wt. %, more than 1 wt. %, more than 5 wt. %, more than 10 wt. %, or more than 15 wt. % plasticizer.

A length of the filament can be measured as a length between each arm, or, for embodiments comprising a single filament wrapping the circumference of the gastric residence system, the entire length of said circumferentially-wrapped filament. Either way, the length of a filament depends on the size of the gastric residence system and the number of arms. For example, for a stellate-shape gastric residence system comprise six arms, the length of a circumferentially-wrapped single filament may be 100-150 mm, 110-140 mm, or 120-130 mm in length. The length of the filament between any two adjacent arms of the six arms may be 18-24 mm or 20-22 mm.

In some embodiments, filaments made from Pellethane tubes may be stretched between two adjacent arms to create tension in the filament between the arms. For a stellate-shape gastric residence system comprise six arms, the length of a circumferentially-wrapped single filament comprising Pellethane tubes may be 90-130 mm or 100-120 mm in length. The length of the filament between any two adjacent arms of the six arms may be 18-22 mm.

The cross-sectional shape of a filament may be any of a variety of shapes including, but not limited to: a circle, an oval, a rectangle, or an annulus. The thickness or diameter of a filament may be 100-1000 microns, preferably 200 to 400 microns. In some embodiments, the thickness or diameter of a filament may be less than 1000 microns, less than 800 microns, less than 600 microns, less than 400 microns, or less than 200 microns. In some embodiments, the thickness or diameter of a filament may be more than 100 microns, more than 200 microns, more than 400 microns, more than 600 microns, or more than 800 microns.

In embodiments comprising a filament having a rectangular cross-section, the width of the filament (i.e., longer side of the rectangular cross-sectional measurement) may be 1-4 mm. In some embodiments, the width may be less than 4 mm, less than 3 mm, or less than 2 mm. In some embodiments, the width may be more than 2 mm, more than 3 mm, or more than 4 mm.

The force required to compress a gastric residence system having a filament may be quantified using a radial compression test, described in detail in the “Testing Methods” section, below. In some embodiments, the force required to compress a gastric residence system having a filament may be 1.25 to 5 times the force required to compress a gastric residence system without a filament to the same compressed diameter. In some embodiments, the force required to compress a gastric residence system having a filament may be less than 5 times, less than 4 times, less than 3 times, or less than 2 times the force required to compress a gastric residence system without a filament to the same compressed diameter. In some embodiments, the force required to compress a gastric residence system having a filament may be more than 1.25 times, more than 2 times, more than 3 times, or more than 4 times the force required to compress a gastric residence system without a filament to the same compressed diameter.

The force required to separate a filament from an arm tip may be quantified using a pullout force test, described in detail in the “Testing Methods” section, below. In some embodiments, the force required to separate a filament from its corresponding arm tip may be 0.5 to 10N or 2 to 6N. In some embodiments, the force required to separate a filament from its corresponding arm tip may be less than 10N, less than 9N, less than 8N, less than 7N, less than 6N, less than 5N, less than 4N, less than 3N, less than 2N, or less than 1N. In some embodiments, the force required to separate a filament from its corresponding arm tip may be more than 0.5N, more than 1N, more than 2N, more than 3N, more than 4N, more than 5N, more than 6N, more than 7N, more than 8N, or more than 9N. In some embodiments, the force required to separate a filament from its corresponding arm tip may decrease the longer the gastric residence system stays in a gastric environment.

In some embodiments, the force required to separate a filament from its corresponding arm tip may depend on the method used to secure the ends of the filament (i.e., knotted, heated, or no secured end). In some embodiments, the force required to separate a filament having knotted ends from its corresponding arm tip may be greater than the force required to separate a filament having heated ends from its corresponding arm tip. In some embodiments, the force required to separate a filament having knotted ends and the force required to separate a filament having heated ends from its corresponding arm tip may be greater than the force required to separate an unmodified filament (i.e., unsecured) from its corresponding arm tip.

### System Polymeric Composition

The choice of the individual polymers for the carrier polymer, coupling polymer, and elastomer influence many properties of the system, such as drug elution rate (dependent on the carrier polymer, as well as other factors), the residence time of the system (dependent on the degradation of any of the polymers, principally the coupling polymers), the uncoupling time of the system if it passes into the intestine (dependent primarily on the enteric degradation rate of the coupling polymer, as discussed herein), and the shelf life of the system in its compressed form (dependent primarily on properties of the elastomer). As the systems will be administered to the gastrointestinal tract, all of the system components should be biocompatible with the gastrointestinal environment.

The rate of elution of drug from the carrier polymer-drug component is affected by numerous factors, including the composition and properties of the carrier polymer, which may itself be a mixture of several polymeric and non-polymeric components; the properties of the drug such as hydrophilicity/hydrophobicity, charge state, pKa, and hydrogen bonding capacity; and the properties of the gastric environment. In the aqueous environment of the stomach, avoiding burst release of a drug (where burst release refers to a high initial delivery of active pharmaceutical ingredient upon initial deployment of the system in the stomach), particularly a hydrophilic drug, and maintaining sustained release of the drug over a period of time of days to one or two weeks is challenging.

The residence time of the systems in the stomach is adjusted by the choice of coupling polymers used in the linker regions. The systems will eventually break down in the stomach, despite the use of enteric coupling polymers, as the mechanical action of the stomach and fluctuating pH will eventually weaken the enteric coupling polymers. Coupling polymers which degrade in a time-dependent manner in the stomach can also be used to adjust the time until the system breaks apart, and hence adjust the residence time. Once the system breaks apart, it passes into the intestines and is then eliminated.

The elastomer used in the systems is central to the shelf life of the systems. When the systems are compressed, the elastomer is subjected to mechanical stress. The stress in turn can cause polymer creep, which, if extensive enough, can prevent the systems from returning to their uncompacted configurations when released from the capsules or other container; this in turn would lead to premature passage of the system from the stomach. Polymer creep can also be temperature dependent, and therefore the expected storage conditions of the systems also need to be considered when choosing the elastomer and other polymer components.

The system components and polymers should not swell, or should have minimal swelling, in the gastric environment. The components should swell no more than about 20%, no more than about 10%, or preferably no more than about 5% when in the gastric environment over the period of residence.

The systems are optionally radiopaque, so that they can be located via abdominal X-ray if necessary. In some embodiments, one or more of the materials used for construction of the system is sufficiently radiopaque for X-ray visualization. In other embodiments, a radiopaque substance is added to one or more materials of the system, or coated onto one or more materials of the system, or are added to a small portion of the system. Examples of suitable radiopaque substances are barium sulfate, bismuth subcarbonate, bismuth oxychloride, and bismuth trioxide. It is preferable that these materials should not be blended into the polymers used to construct the gastric residence system, so as not to alter drug release from the carrier polymer, or desired properties of other system polymers. Metal striping or tips on a small portion of the system components can also be used, such as tungsten.

### Methods of Manufacture and Methods of Treatment

Following is a description of various methods of manufacturing and methods of treatment. In particular, included is a detailed description of: Manufacture/assembly of system: three-dimensional printing; Manufacture/assembly of system: co-extrusion; Agent particle size and milling; Methods of Manufacture of Carrier Polymer-Agent (or Agent Salt) Components; Manufacture/assembly of system: affixing arms to central elastomer; Manufacture/assembly of system; Methods of Preparing Gastric Residence Systems Having Flexible Arms; Methods of treatment using the gastric residence systems; and Kits and Articles of Manufacture.

### Manufacture/Assembly of System: Three-Dimensional Printing

Three-dimensional printing of components of the gastric residence system, such as arm or arm segments, is performed using commercially-available equipment. Three-dimensional printing has been used for pharmaceutical preparation; see Khaled et al., “Desktop 3D printing of controlled release pharmaceutical bilayer tablets,” International Journal of Pharmaceutics 461:105-111 (2014); U.S. Pat. No. 7,276,252; Alhnan et al., “Emergence of 3D Printed Dosage Forms: Opportunities and Challenges,” Pharm. Res., May 18, 2016, PubMed PMID: 27194002); Yu et al., “Three-dimensional printing in pharmaceutics: promises and problems,” J. Pharm. Sci. 97(9):3666-3690 (2008); and Ursan et al., “Three-dimensional drug printing: A structured review,” J. Am. Pharm. Assoc. 53(2):136-44 (2013).

The initial feedstocks for three-dimensional printing are polymers or polymer blends (e.g. enteric polymers, time-dependent polymers, or blends of one or more of an agent, an agent salt, a drug, an excipient, etc., with a carrier polymer, enteric polymers, or time-dependent polymers). The polymer or ingredients which are to be used for one region of the segment or arm to be manufactured are mixed and pelletized using hot melt extrusion. The polymer or blended polymer material is extruded through a circular die, creating a cylindrical fiber which is wound around a spool.

Multiple spools are fed into the 3D printer (such as a Hyrel Printer, available from Hyrel 3D, Norcross, Ga., United States), to be fed into their representative print heads. The print heads heat up and melt the material at the nozzle, and lay down a thin layer of material (polymer or polymer blend) in a specific position on the piece being manufactured. The material cools and hardens within seconds, and the next layer is added until the complete structure is formed. The quality of the dosage form is dependent on the feed rate, nozzle temperature, and printer resolution; feed rate and nozzle temperature can be adjusted to obtain the desired quality.

Three-dimensional printing can be used to manufacture individual arms, or segments of arms. Three-dimensional printing can also be used to prepare a bulk configuration, such as a consolidated “slab,” similar to that prepared by co-extrusion methods described herein. The bulk configuration can be cut into individual pieces (that is, individual arms or individual segments) as needed.

In some embodiments of the invention, producing an entire arm of the gastric residence system by three-dimensional printing of the arm is contemplated. In some embodiments of the invention, producing a segment of an arm of the gastric residence system by three-dimensional printing of the segment of an arm is contemplated. In some embodiments, an arm or a segment thereof is produced by three-dimensional printing of adjacent portions of carrier polymer-agent or polymer-agent salt blend and linker material in a bulk configuration, such as a slab configuration. The three-dimensional printing can be followed by cutting the bulk configuration into pieces which have the desired shape of the arm or segment thereof. The three-dimensional printing can be followed by compression molding of portions of the bulk configuration into pieces which have the desired shape of the arm or segment thereof.

Three-dimensional printing is often accomplished by feeding a rod or fiber of a solid material to a print head, where it is melted and deposited with subsequent solidification, in a technique known as fused deposition modeling (sometimes also called extrusion deposition); see U.S. Pat. Nos. 5,121,329 and 5,340,433. The methods described herein for the manufacture of carrier polymer-drug components can also be used to manufacture feed material, which can be used in the manufacture via three-dimensional printing of components of the gastric residence systems.

### Manufacture/Assembly of System: Co-Extrusion

Components of the gastric residence systems can be manufactured by co-extrusion. Most of the various configurations for the segments discussed herein, such as the “islands-in-the-sea” configurations, can be made by either three-dimensional printing or co-extrusion. However, co-extrusion is less expensive, and can be run as a continuous process, as opposed to three-dimensional printing, which is generally run as a batch process.

Co-extrusion of the “islands-in-the-sea” configuration is used in the textile industry and for production of fiber optics, but has rarely been applied in biomedical systems. See U.S. Pat. Nos. 3,531,368; 3,716,614; 4,812,012; and Haslauer et al., J. Biomed. Mater. Res. B Appl. Biomater. 103(5):1050-8 (2015)).

Co-extrusion of components of the gastric residence system, such as an arm, or a segment of an arm, can be performed using commercially-available equipment, combined with customized co-extruder plumbing and customized dies for the desired configuration. The initial feedstocks for co-extrusion are polymers or polymer blends (e.g. enteric polymers, time-dependent polymers, or blends of one or more of an agent, an agent salt, a drug, an excipient, etc., with a carrier polymer, enteric polymers, or time-dependent polymers). The polymer or ingredients which are to be used for one region of the segment or arm to be manufactured are mixed and pelletized using hot melt extrusion. The polymer pellets thus formed are placed into hoppers above single screw extruders and dried to remove surface moisture. Pellets are gravimetrically fed into individual single-screw extruders, where they are melted and pressurized for co-extrusion.

The appropriate molten polymers are then pumped through custom designed dies with multiple channels where they form the required geometry. The composite polymer block is cooled (water-cooled, air-cooled, or both) and cut or stamped into the desired shape, including, but not limited to, such shapes as triangular prisms, rectangular prisms, or cylinder sections (pie-shaped wedges).

In some embodiments of the invention, producing an entire arm of the gastric residence system by co-extruding the arm is contemplated. In some embodiments of the invention, producing a segment of an arm of the gastric residence system by co-extruding the segment of an arm is contemplated. In some embodiments, an arm or a segment thereof is produced by co-extruding adjacent portions of carrier polymer-agent or carrier polymer-agent salt blend and linker material in a bulk configuration, such as a slab configuration. The co-extruding can be followed by cutting the bulk configuration into pieces which have the desired shape of the arm or segment thereof. The co-extruding can be followed by compression molding of portions of the bulk configuration into pieces which have the desired shape of the arm or segment thereof.

In some embodiments, an arm or a segment thereof is produced by co-extruding adjacent portions of carrier polymer-agent or carrier polymer-agent salt blend and linker material in a bulk configuration, such as a slab configuration, while also co-extruding an additional polymer or polymers within the carrier polymer-agent or carrier polymer-agent salt blend, the linker material, or both the carrier polymer-agent (or agent salt) blend and the linker material. The co-extruding the additional polymer or polymers within the carrier polymer-agent or carrier polymer-agent salt blend, the linker material, or both the carrier polymer-agent (or agent salt) blend and the linker material can be performed in an islands-in-the-sea configuration. The co-extruding can be followed by cutting the bulk configuration into pieces which have the desired shape of the arm or segment thereof. The co-extruding can be followed by compression molding of portions of the bulk configuration into pieces which have the desired shape of the arm or segment thereof.

### Agent Particle Size and Milling

Control of particle size used in the gastric residence systems is important for both optimal release of agent and mechanical stability of the systems. The particle size of the agents affects the surface area of the agents available for dissolution when gastric fluid permeates the carrier polymer-agent segments of the system. Also, as the arms of the systems are relatively thin in diameter (for example, 1 millimeter to 5 millimeters), the presence of a particle of agent of a size in excess of a few percent of the diameter of the arms will result in a weaker arm, both before the agent elutes from the device, and after elution when a void is left in the space formerly occupied by the agent particle. Such weakening of the arms is disadvantageous, as it may lead to premature breakage and passage of the system before the end of the desired residence period.

In one embodiment, the agent particles used for blending into the carrier polymer-agent components are smaller than about 100 microns in diameter. In another embodiment, the agent particles are smaller than about 75 microns in diameter. In another embodiment, the agent particles are smaller than about 50 microns in diameter. In another embodiment, the agent particles are smaller than about 40 microns in diameter. In another embodiment, the agent particles are smaller than about 30 microns in diameter. In another embodiment, the agent particles are smaller than about 25 microns in diameter. In another embodiment, the agent particles are smaller than about 20 microns in diameter. In another embodiment, the agent particles are smaller than about 10 microns in diameter. In another embodiment, the agent particles are smaller than about 5 microns in diameter.

In one embodiment, at least about 80% of the agent particles used for blending into the carrier polymer-agent components are smaller than about 100 microns in diameter. In another embodiment, at least about 80% of the agent particles are smaller than about 75 microns in diameter. In another embodiment, at least about 80% of the agent particles are smaller than about 50 microns in diameter. In another embodiment, at least about 80% of the agent particles are smaller than about 40 microns in diameter. In another embodiment, at least about 80% of the agent particles are smaller than about 30 microns in diameter. In another embodiment, at least about 80% of the agent particles are smaller than about 25 microns in diameter. In another embodiment, at least about 80% of the agent particles are smaller than about 20 microns in diameter. In another embodiment, at least about 80% of the agent particles are smaller than about 10 microns in diameter. In another embodiment, at least about 80% of the agent particles are smaller than about 5 microns in diameter.

In one embodiment, at least about 80% of the mass of the agent particles used for blending into the carrier polymer-agent components have sizes between about 1 micron and about 100 microns in diameter. In another embodiment, at least about 80% of the mass of the agent particles have sizes between about 1 micron and about 75 microns in diameter. In another embodiment, at least about 80% of the mass of the agent particles have sizes between about 1 micron and about 50 microns in diameter. In another embodiment, at least about 80% of the mass of the agent particles have sizes between about 1 micron and about 40 microns in diameter. In another embodiment, at least about 80% of the mass of the agent particles have sizes between about 1 micron and about 30 microns in diameter. In another embodiment, at least about 80% of the mass of the agent particles have sizes between about 1 micron and about 25 microns in diameter. In another embodiment, at least about 80% of the mass of the agent particles have sizes between about 1 micron and about 20 microns in diameter. In another embodiment, at least about 80% of the mass of the agent particles have sizes between about 1 micron and about 10 microns in diameter. In another embodiment, at least about 80% of the mass of the agent particles have sizes between about 1 micron and about 5 microns in diameter.

In one embodiment, at least about 80% of the mass of the agent particles used for blending into the carrier polymer-agent components have sizes between about 2 microns and about 100 microns in diameter. In another embodiment, at least about 80% of the mass of the agent particles have sizes between about 2 microns and about 75 microns in diameter. In another embodiment, at least about 80% of the mass of the agent particles have sizes between about 2 microns and about 50 microns in diameter. In another embodiment, at least about 80% of the mass of the agent particles have sizes between about 2 microns and about 40 microns in diameter. In another embodiment, at least about 80% of the mass of the agent particles have sizes between about 2 microns and about 30 microns in diameter. In another embodiment, at least about 80% of the mass of the agent particles have sizes between about 2 microns and about 25 microns in diameter. In another embodiment, at least about 80% of the mass of the agent particles have sizes between about 2 microns and about 20 microns in diameter. In another embodiment, at least about 80% of the mass of the agent particles have sizes between about 2 microns and about 10 microns in diameter. In another embodiment, at least about 80% of the mass of the agent particles have sizes between about 2 microns and about 5 microns in diameter.

In one embodiment, at least about 80% of the mass of the agent particles used for blending into the carrier polymer-agent components have sizes between about 5 microns and about 100 microns in diameter. In another embodiment, at least about 80% of the mass of the agent particles have sizes between about 5 microns and about 75 microns in diameter. In another embodiment, at least about 80% of the mass of the agent particles have sizes between about 5 microns and about 50 microns in diameter. In another embodiment, at least about 80% of the mass of the agent particles have sizes between about 5 microns and about 40 microns in diameter. In another embodiment, at least about 80% of the mass of the agent particles have sizes between about 5 microns and about 30 microns in diameter. In another embodiment, at least about 80% of the mass of the agent particles have sizes between about 5 microns and about 25 microns in diameter. In another embodiment, at least about 80% of the mass of the agent particles have sizes between about 5 microns and about 20 microns in diameter. In another embodiment, at least about 80% of the mass of the agent particles have sizes between about 5 microns and about 10 microns in diameter.

The particle size of the agents can be readily adjusted by milling. Several milling techniques are available to reduce larger particles to smaller particles of desired size. Fluid energy milling is a dry milling technique which uses inter-particle collisions to reduce the size of particles. A type of fluid energy mill called an air jet mill shoots air into a cylindrical chamber in a manner so as to maximize collision between agent particles. Ball milling utilizes a rolling cylindrical chamber which rotates around its principal axis. The agent and grinding material (such as steel balls, made from chrome steel or CR-NI steel; ceramic balls, such as zirconia; or plastic polyamides) collide, causing reduction in particle size of the agent. Ball milling can be performed in either the dry state, or with liquid added to the cylinder where the agent and the grinding material are insoluble in the liquid. Further information regarding milling is described in the chapter by R. W. Lee et al. entitled “Particle Size Reduction” in Water-Insoluble Drug Formulation, Second Edition (Ron Liu, editor), Boca Raton, Fla.: CRC Press, 2008; and in the chapter by A. W. Brzeczko et al. entitled “Granulation of Poorly Water-Soluble Drugs” in Handbook of Pharmaceutical Granulation Technology, Third Edition (Dilip M. Parikh, editor), Boca Raton, Fla.: CRC Press/Taylor & Francis Group, 2010 (and other sections of that handbook). Fluid energy milling (i.e., air jet milling) is a preferred method of milling, as it is more amenable to scale-up compared to other dry milling techniques such as ball milling.

Substances can be added to the agent material during milling to assist in obtaining particles of the desired size, and minimize aggregation during handling. Silica (silicon dioxide, SiO2) is a preferred milling additive, as it is inexpensive, widely available, and non-toxic. Other additives which can be used include silica, calcium phosphate, powdered cellulose, colloidal silicon dioxide, hydrophobic colloidal silica, magnesium oxide, magnesium silicate, magnesium trisilicate, talc, polyvinylpyrrolidone, cellulose ethers, polyethylene glycol, polyvinyl alcohol, and surfactants. In particular, hydrophobic particles less than 5 microns in diameter are particularly prone to agglomeration, and hydrophilic additives are used when milling such particles. A weight/weight ratio of about 0.1% to about 5% of milling additive, such as silica, can be used for fluid milling or ball milling, or about 0.1% to about 4%, about 0.1% to about 3%, about 0.1% to about 2%, about 0.1% to about 1%, about 1% to about 5%, about 1% to about 4%, about 1% to about 3%, about 1% to about 2%, or about 0.1%, about 0.5%, about 1%, about 2%, about 3%, about 4% or about 5%.

After milling, particles can be passed through meshes of appropriate size to obtain particles of the desired size. To obtain particles of a desired maximum size, particles are passed through a mesh with holes of the maximum size desired; particles which are too large will be retained on the mesh, and particles which pass through the mesh will have the desired maximum size. To obtain particles of a desired minimum size, particles are passed through a mesh with holes of the minimum size desired; particles which pass through the mesh are too small, and the desired particles will be retained on the mesh.

### Methods of Manufacture of Carrier Polymer-Agent (or Agent Salt) Components

Blending temperatures for incorporation of the agent (or a pharmaceutically acceptable salt thereof) into polymeric matrices typically range from about 80° C. to about 120° C., although higher or lower temperatures can be used for polymers which are best blended at temperatures outside that range. When agent (or salt thereof) particles of a particular size are used, and it is desired that the size of the particles be maintained during and after blending, blending can be done at temperatures below the melting point of the agent (or salt thereof), so as to maintain the desired size of the particles. Otherwise, temperatures can be used which melt both the polymer and the agent (or salt thereof). Blending temperatures should be below the degradation temperature of the agent (or salt thereof). In one embodiment, less than about 2% of the agent (or salt thereof) is degraded during manufacture. In one embodiment, less than about 1.5% of the agent (or salt thereof) is degraded during manufacture. In one embodiment, less than about 1% of the agent (or salt thereof) is degraded during manufacture. In one embodiment, less than about 0.75% of the agent (or salt thereof) is degraded during manufacture. In one embodiment, less than about 0.5% of the agent (or salt thereof) is degraded during manufacture. In one embodiment, less than about 0.4% of the agent (or salt thereof) is degraded during manufacture. In one embodiment, less than about 0.3% of the agent (or salt thereof) is degraded during manufacture. In one embodiment, less than about 0.2% of the agent (or salt thereof) is degraded during manufacture. In one embodiment, less than about 0.15% of the agent (or salt thereof) is degraded during manufacture. In one embodiment, less than about 0.1% of the agent (or salt thereof) is degraded during manufacture. In one embodiment, less than about 0.05% of the agent (or salt thereof) is degraded during manufacture. In one embodiment, less than about 0.04% of the agent (or salt thereof) is degraded during manufacture. In one embodiment, less than about 0.03% of the agent (or salt thereof) is degraded during manufacture. In one embodiment, less than about 0.02% of the agent (or salt thereof) is degraded during manufacture. In one embodiment, less than about 0.01% of the agent (or salt thereof) is degraded during manufacture.

Hot melt extrusion can be used to prepare the carrier polymer-agent (or agent salt) components. Single-screw or, preferably, twin-screw systems can be used. As noted, if it is desired that the size of the particles be maintained during and after blending, carrier polymers should be used which can be melted at temperatures which do not degrade the agent or salt thereof. Otherwise, temperatures can be used which melt both the polymer and the agent or salt thereof.

Melting and casting can also be used to prepare the carrier polymer-agent (or salt thereof) components. The carrier polymer and agent (or salt thereof), and any other desired components, are mixed together. The carrier polymer is melted and the melt is mixed so that the agent (or salt thereof) particles are evenly distributed in the melt, poured into a mold, and allowed to cool.

Solvent casting can also be used to prepare the carrier polymer-agent (or salt thereof) components. The polymer is dissolved in a solvent, and particles of agent (or salt thereof) are added. If the size of the agent (or salt thereof) particles are to be maintained, a solvent should be used which does not dissolve the agent (or salt thereof) particles, so as to avoid altering the size characteristics of the particles; otherwise, a solvent which dissolves both the polymer and agent (or salt thereof) particles can be used. The solvent-carrier polymer-agent (or salt thereof) particle mixture (or solvent-carrier particle-agent/agent salt solution), is then mixed to evenly distribute the particles (or thoroughly mix the solution), poured into a mold, and the solvent is evaporated.

### Manufacture/Assembly of System: Affixing Arms to Central Elastomer

For a stellate gastric residence system, such as that shown in FIG. 1A, the arms of the gastric residence system can be affixed to the central elastomer in a number of ways. The central polymer can be cast or molded with short “asterisk” arms, and a linker polymer can be used to affix the arms to the asterisk arms of the central elastomer. Alternatively, the central elastomer can be formed in a mold into which the proximal ends of the arms protrude. The elastomer sets, cures, or otherwise hardens into its desired form with a portion of the arms extending into the body of the central elastomer. Alternatively, the central elastomer can be prepared with cavities into which the arms can be firmly inserted.

The invention thus includes a method of making a gastric residence system, comprising preparing at least three arms formed from a material comprising any drug-carrier polymer-excipient formulation as disclosed herein; and attaching the arms to a central elastomer to form a gastric residence system. The arms can comprise at least one segment with a release rate-controlling polymer film. The arms of the gastric residence system project radially from the central elastomer, such as in a “hub and spoke” arrangement. A preferred number of arms is six. However, stellate systems with three, four, five, seven, or eight arms can also be used.

In some embodiments, arms comprising any carrier polymer-agent formulation can be heat-welded, solvent-welded, or otherwise affixed to other elements, including disintegrating matrices, coupling polymers, or interfacing polymers, which are then affixed to a central elastomer. In some embodiments, the arms are directly affixed to a central elastomer. Disintegrating matrices, coupling polymers, or interfacing polymer segments can be welded or otherwise affixed to the central elastomer prior to affixing the arms.

In some embodiments, arms comprising any drug-carrier polymer-excipient formulation as disclosed herein can be heat-welded to polycaprolactone segments, such as short polycaprolactone “asterisk” arms affixed to a central elastomer. Linker segments can be welded to the short “asterisk” arms prior to affixing the drug-carrier polymer-excipient formulation arms. Heat welding of drug-carrier polymer-excipient formulation arms to MW 80,000 PCL segments at temperatures between 140° C. to 170° C., followed by cooling for 24 hours at 8° C., resulted in stronger welds. Thus, in one embodiment, attaching the arms comprising any drug-carrier polymer-excipient formulation as disclosed herein to a central elastomer to form a gastric residence system, can comprise heat-welding the arms to other system components, such as asterisk arms or other segments comprising at least about 90%, at least about 95%, or at least about 99% polycaprolactone (such as MW 80,000 PCL), at a temperature between about 140° C. to about 170° C., followed by cooling of the welded members attached to other system components for about 12 to about 48 hours at a temperature of about 2° C. to about 14° C., such as about 5° C. to about 10° C., or about 8° C. The other system components can alternatively be linker elements.

### Manufacture/Assembly of System

Once the arms of the gastric residence system have been affixed to the central elastomer, the system is ready to be folded into its compacted configuration and placed into a capsule for storage, transport, and eventual administration. The system can be folded in an automated mechanical process, or by hand, and placed into a capsule of the appropriate size and material. More detail regarding manufacture and assembly of gastric residence systems, and of packaging the gastric residence system into capsules, can be found in International Patent Application Nos. WO 2015/191920, WO 2015/191925, WO 2017/070612, WO 2017/100367, and PCT/US2017/034856.

### Methods of Preparing Gastric Residence Systems Having Flexible Arms

As described in detail herein, flexible arms for gastric residence systems having flexible arms may comprise two portions—a first segment comprising a first polymer composition and a second segment comprising a second polymer composition.

In some embodiments, the first segment may be welded to the second segment. In some embodiments, the arm may be extruded or prepared using injection molding.

### Methods of Treatment Using the Gastric Residence Systems

The gastric residence systems can be used to treat conditions requiring administration of a drug or agent over an extended period of time. In a preferred embodiment, a gastric residence system is administered to a human. For long-term administration of agents or drugs which are taken for months, years, or indefinitely, administration of a gastric residence system periodically, such as once weekly or once every two weeks can provide substantial advantages in patient compliance and convenience. Accordingly, the gastric residence systems of the invention can be administered once every three days, once every five days, once weekly, once every ten days, or once every two weeks. The administration frequency is timed to coincide with the designed gastric residence period of the gastric residence system which is administered, so that at about the same time that a gastric residence system passes out of the stomach after its residence period, a new gastric residence system is administered.

Once a gastric residence system has been administered to a patient, the system provides sustained release of agent or drug over the period of gastric retention. After the period of gastric retention, the system degrades and passes out of the stomach. Thus, for a system with a gastric retention period of one week, the patient will swallow (or have administered to the stomach via other methods) a new system every week. Accordingly, in one embodiment, a method of treatment of a patient with a gastric retention system of the invention having a gastric residence period of a number of days D (where D-days is the gastric residence period in days), over a total desired treatment period T-total (where T-total is the desired length of treatment in days) with the agent or drug in the system, comprises introducing a new gastric residence system every D-days into the stomach of the patient, by oral administration or other methods, over the total desired treatment period. The number of gastric residence systems administered to the patient will be (T-total) divided by (D-days). For example, if treatment of a patient for a year (T-total=365 days) is desired, and the gastric residence period of the system is 7 days (D-days=7 days), approximately 52 gastric residence systems will be administered to the patient over the 365 days, as a new system will be administered once every seven days.

Alternatively, the patient can swallow (or have administered to the stomach via other methods) a new gastric residence system at the end of the effective release period of the gastric residence system. The “effective release period” or “effective release time” is the time over which the gastric residence system releases an effective amount of the agent contained in the system. Accordingly, in one embodiment, a method of treatment of a patient with a gastric residence system of the invention having an effective release period of a number of days E (where E-days is the effective release period in days), over a total desired treatment period T-total (where T-total is the desired length of treatment in days) with the agent in the system, comprises introducing a new gastric residence system every E-days into the stomach of the patient, by oral administration or other means, over the total desired treatment period. The number of gastric residence systems administered to the patient will be (T-total) divided by (E-days). For example, if treatment of a patient for a year (T-total=365 days) is desired, and the effective release period of the system is 7 days (E-days=7 days), approximately 52 gastric residence systems will be administered to the patient over the 365 days, as a new system will be administered once every seven days.

### Kits and Articles of Manufacture

Also provided herein are kits for treatment of patients with the gastric residence systems of the invention. The kit may contain, for example, a sufficient number of gastric residence systems for periodic administration to a patient over a desired total treatment time period. If the total treatment time in days is (T-total), and the gastric residence systems have a residence time of (D-days), then the kit will contain a number of gastric residence systems equal to ((T-total) divided by (D-days)) (rounded to an integral number), for administration every D-days. Alternatively, if the total treatment time in days is (T-total), and the gastric residence systems have an effective release period of (E-days), then the kit will contain a number of gastric residence systems equal to ((T-total) divided by (E-days)) (rounded to an integral number), for administration every E-days. The kit may contain, for example, several gastric residence systems in containers (where the containers may be capsules) and may optionally also contain printed or computer readable instructions for dosing regimens, duration of treatment, or other information pertinent to the use of the gastric residence systems and/or the agent or drug contained in the gastric residence systems. For example, if the total treatment period prescribed for the patient is one year, and the gastric residence system has a residence time of one week or an effective release period of one week, the kit may contain 52 capsules, each capsule containing one gastric residence system, with instructions to swallow one capsule once a week on the same day (e.g., every Saturday).

Articles of manufacture, comprising a sufficient number of gastric residence systems for periodic administration to a patient over a desired total treatment time period, and optionally comprising instructions for dosing regimens, duration of treatment, or other information pertinent to the use of the gastric residence systems and/or the agent or drug contained in the gastric residence systems, are also included in the invention. The articles of manufacture may be supplied in appropriate packaging, such as dispensers, trays, or other packaging that assists the patient in administration of the gastric residence systems at the prescribed interval.

### Testing Methods

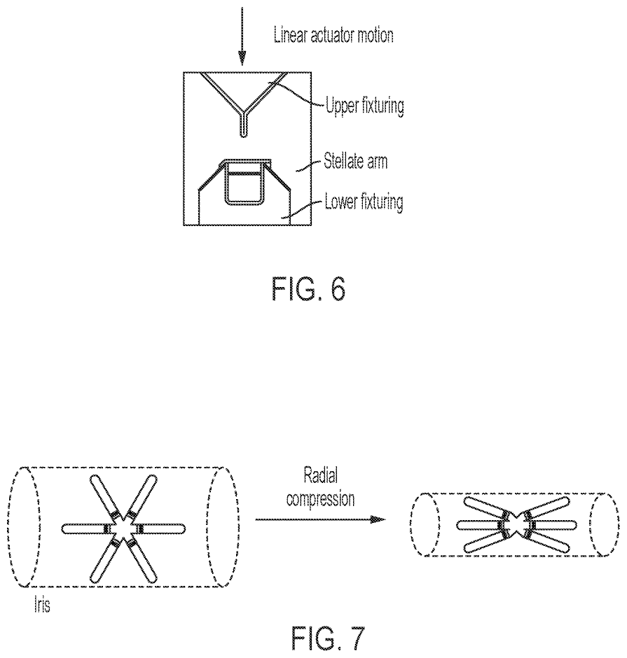

3-Point Bending Test: FIG. 6 shows how the stiffness of a material may be tested. Specifically, FIG. 6 shows how the stiffness of a material may be measured using a 3-point bending test. The 3-point bending test shown in FIG. 6 is based on the ASTM standard 3-point bending test (ASTM D790) but is modified to accommodate the size and shape of gastric residence system arms. An Instron machine 3342 Series and a custom-made base for triangular shapes were used to evaluate the 3-Point Bend Young's Modulus and/or flexural strength in a variety of incubation media, after several incubation cycles, and at room temperature or body temperature (37-40 degrees C.).

As shown, the 3-point bending test includes linear actuator motor 610, upper fixture 612, and lower fixture 614 comprising two raised supports. Samples (e.g., arm 616) were prepared by holt-melt extruding a uniform rod of triangular cross-section (equilateral triangles having sides of 3.3 mm in length) and 12-20 mm in length.

To test the stiffness of arm 616, arm 616 is placed on lower fixture 614 such the distal end of arm 616 is resting atop one raised support and the proximal end of arm 616 is resting atop the other of the two raised supports. The two raised supports of lower fixture 614 were 9 mm apart. Linear actuator motor 610 was an Instron mechanical testing machine, to which upper fixture 612 was attached.

To test the stiffness of arm 616, upper fixture 612 was lowered onto a flat top of arm 616 and the force (N/mm) was measured using a load cell. Upper fixture 612 comprised a 1 mm wide rounded element that came in contact with arm 616 during testing. Thus, a flat side of arm 616 was compressed with a semi-circular element having a 1 mm radius of curvature. Stiffness may be reported as the slope of the linear region of the force-displacement curve generated by the load cell in units of N/mm.

Radial Force Compression Test: FIG. 7 shows a radial force compression test using an iris mechanism. Specifically, gastric residence system 702 depicted in FIG. 7 comprises relatively stiff arms; however, the radial force compression test shown in the figure was used to test gastric residence systems having relatively flexible arms as described herein. The instrument (i.e., iris tester) used to measure radial force compression was a Blockwise Model TTR2 Tensile Testing Machine with Model RLU124 Twin-Cam™ Radial Compression Station, 60 mm D×124 mm L.

The gastric residence system that was measured was placed in the iris tester such that the plane of the gastric residence system was parallel to the axis of the iris cylinder. Four arm tips were placed in contact with the interior wall of the iris tester (in the case of gastric residence systems comprising six arms), where two arms are angled upwards and two arms are angled downwards. Two additional arms were oriented parallel to the axis of the iris cylinder.

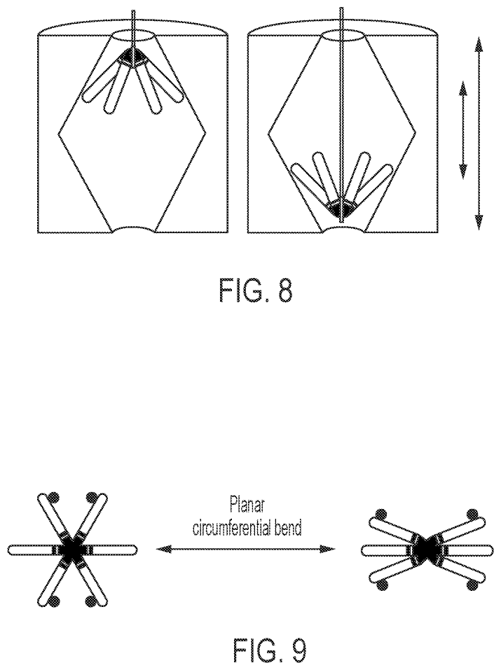

Double Funnel Durability Test: FIG. 8 depicts a double funnel test used to quantify the durability of a gastric residence system. The durability of a gastric residence system can help prevent the premature breaking or weakening due to repeated gastric wave/forces (and early passage through the pylorus) of a gastric residence system. As shown, gastric residence system 802 is gripped at its center (i.e., core) by ring 820 attached to a linear actuator. Gastric residence system 802 is repeated moved upwards and downwards into cone-shaped cavities 822, causing the arms of gastric residence system 802 to bend back and forth with reference to the core. This upwards and downwards motion is repeated for hundreds of cycles or until gastric residence system 802 breaks. Different specific failure modes may include a breakage at a connection point (e.g., arm-to-core or first segment-to-second segment) or tearing of the silicone core. The number of cycles to failure and the force required to bend gastric residence system 802 may be quantified. The test may be performed with gastric residence system 802 submerged in aqueous media (e.g., simulated gastric fluid) and at body temperature.

Planar Circumferential Bend Durability Test: FIG. 9 shows another test that may be used to quantify the durability and failure mode of a gastric residence system. In particular, the planar circumferential bend durability test can test gastric residence system 902 by positioning it onto a puck having four grips 930 in contact with arms of gastric residence system 902. Grips 930 are connected to a rotational actuator that applies force to the arms in a circumferential motion. This motion causes the arms to spread within the plane of gastric residence system 902. The motion is repeated for hundreds of cycles or until gastric residence system 902 breaks. Different specific failure modes may include a breakage at a connection point (e.g., arm-to-core or first segment-to-second segment) or tearing of the silicone core. The number of cycles to failure and the force required to bend gastric residence system 902 may be quantified. The test may be performed with gastric residence system 902 submerged in aqueous media (e.g., simulated gastric fluid) and at body temperature.

Pullout Force Test: The adhesion strength of a filament for a gastric residence system can be tested using a pullout force test. As described previously, the filament may be attached to a distal end of an arm. In cases where a single filament connects more than two arms, the filament may be connected to the distal end of each arm to prevent translation of the arm along the filament when the gastric residence system is bent by gastric forces. Thus, the pullout force test described herein can quantify the amount of force required to separate the filament from the distal end of an arm.

Gastric residence systems having six arms and a filament were prepared and the arms were isolated by cutting the elastomeric core into six parts. The filament was cut between each arm. The tensile force required to pull the filament out of each arm tip was measured using an Instron 3340 Series Universal Testing System by gripping the base of the arm and one end of the filament.

Melt Flow Index (MFI): The melt flow index (MFI) is a measurement of viscosity at low shear, measured in grams of material that flow through a die in 10 minutes at a set temperature and applied weight. These measurements are performed using a Ray-Ran 6MPCA Advanced Melt Flow System, with a weight of 2.16 kg (but can be with a range of standardized weights) and following Procedure A of ASTM D1238 “Standard Test Method for Melt Flow Rates of Thermoplastics by Extrusion Plastometer.”

Tensile Test: An Instron machine having custom-made grips (Figure C) can be used to evaluate the ultimate tensile strength (UTS) of the bond between any combination of stellate components: (1) in a variety of incubation media; (2) at several times of incubation; and (3) at room temperature or body temperature (37-40° C.). A low ultimate tensile strength indicates a potential failure point in the stellate. Using formulation and process optimization, tensile strength can be maximized for ideal stellate performance.

For testing stellate arms with a triangular cross-section, custom-made grips can be used having one flat plate and one notched plate. The apex of the triangular arm sits in the notch, in order to distribute the pressure from the plates more evenly across the three lengthwise faces of the triangular arm.

Tensile testing was performed using an Instron 3342 Series. A series of hot-melt extruded, thermally bonded equilateral triangular prisms with a 3.33 mm triangular base is gripped using pneumatic actuation. The crosshead moves upward at 5-500 mm/minute depending on the elasticity of the materials tested. The instrument records Force (N) v. Displacement (mm), and the maximum force is divided by the cross-sectional area at the interface to calculate ultimate tensile strength (stress).

Drug Release Rate Test: Release rate of drug is tested in fasted-state simulated gastric fluid (FaSSGF). FaSSGF was prepared as follows, according to the manufacturer's instructions (biorelevant.com). 975 mL deionized water and 25 mL of 1N hydrochloric acid were mixed in a 1 L glass media bottle. The pH was adjusted to 1.6 using 1N HCl or NaOH as needed. 2.0 grams of NaCl was added and mixed in. Just before use, 60 mg of Biorelevant powder was mixed into the solution. The composition of FaSSGF was taurocholate (0.08 mM), phospholipids (0.02 mM), sodium (34 mM), chloride (59 mM). Carrier polymer-agent compositions were formed into drug-loaded polymer arms by blending polymer powder and active pharmaceutical ingredient, and extruding. Arms were coated with release rate-modulating polymer films by dissolving the film polymer in an appropriate solvent, typically ethyl acetate or acetone, and pan-coating or dip-coating the arm in the solution of film polymer. Coated arms are then placed in a vessel containing FaSSGF, incubated at 37° C., and typically sampled at least four times over a seven-day period. Drug content was measured by HPLC. Samples were stored for no more than 3 days at 4° C. prior to analysis. At each measurement time point, in order to maintain sink conditions, the entire volume of release media was replaced with fresh solution pre-equilibrated to 37° C.

### EXAMPLES

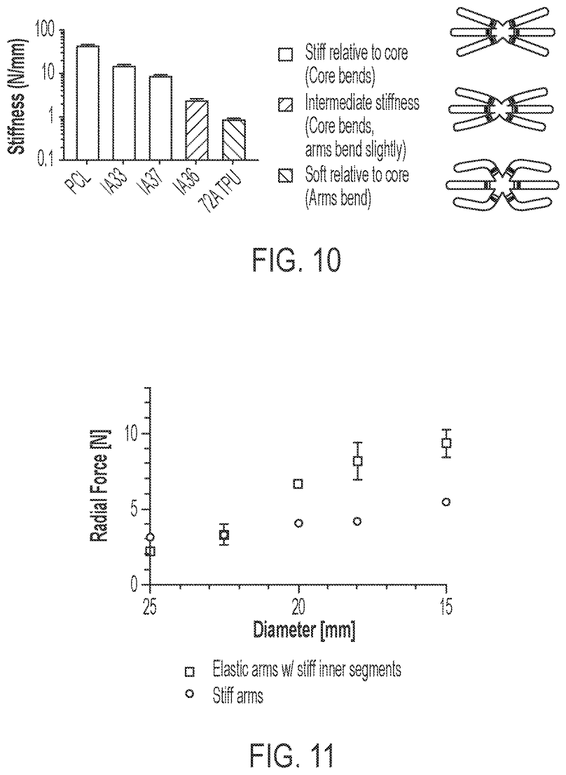

Example 1: FIG. 10 shows stiffness data of five different arms as measured using the 3-point bending test described in detail above and depicted in FIG. 6. The five different arms tested include arms comprising polycaprolactone, (PCL), polycaprolactone combined with soluble materials (IA33, IA27, and IA36), and thermoplastic polyurethane having a durometer of 72A (72A TPU). The formulations of the different arms are provided in Table 1, below:

| | | Hydrated |
|---|---|---|
| Formulation or | | Stiffness |
| Material Name | Composition | (N/mm) |
| SS09 | 30% Mannitol, 70% PCL | 21.8 |
| IA30 | 35% VA64, 1.5% P407, 63.5% PCL | 7.0 |
| IA33 | 20% VA64, 1.5% P407, 78.5% PCL | 14.4 |
| IA36 | 42% VA64, 15% PEO, 3% P407, 40% PCL | 3.0 |
| IA37 | 32% VA64, 1.5% P407, 66.5% PCL | 10.3 |

Table 1, above, includes the following materials: mannitol, polycaprolactone (PCL), copovidone (VA64, Kollidon VA64), poloxamer P407 (P407), and polyethylene oxide 100 kDa (PEO).

As shown in the figure, the pure polycaprolactone arms exhibited the greatest stiffness and the thermoplastic polyurethane arms exhibited the least stiffness values. The three arms comprising polycaprolactone and soluble materials showed a moderate level of stiffness in comparison. As discussed above, these materials (polycaprolactone mixed with soluble materials) lose stiffness when exposed to an aqueous environment and the soluble materials are hydrated.

Additionally, the relative stiffness of each material tested was reported. This was determined using an eyeball test (i.e., how easily the arms bend when the gastric residence system is compressed). As shown, the white bars represent relatively stiff arms (when the gastric residence system is compressed, the core bends and the arms remain straight), the hatched bar represents an arm of intermediate stiffness (core bends and arms bend slightly), and the shaded bar represents relatively soft or flexible arms (arms bend before the core bends).

Example 2: FIG. 11 shows radial force data for two different types of gastric residence systems. Both gastric residence systems having flexible arms (comprising a first segment and a second segment) and gastric residence systems having stiff arms were tested using the radial force compression test depicted in FIG. 4 and described in detail above. In particular, the gastric residence systems that were tested comprised 50A silicone cores. The gastric residence system having stiff arms comprised arms made of polycaprolactone. The gastric residence system comprising flexible arms comprised arms made of a stiff blend of PLGA, PLA, HPMCAS, and TPU (first segment) and TPU (Pathways 72A) (second segment).

As shown, for both types of gastric residence systems that were tested, the force increased as the diameter of the iris tester decreased. At relatively large iris tester diameters (i.e., compression diameters of 20 mm and greater), the gastric residence system having flexible arms compressed with less force.

However, the results also reveal that the force required to compress the gastric residence system having the flexible arms to an iris tester diameter of 20 mm and less was markedly greater than the force required to compress the gastric residence system having stiff arms to an iris tester diameter of 20 mm or less. Accordingly, this indicates that the force required to compress a gastric residence system having flexible arms to a bended configuration small enough to pass through a pylorus (i.e., an opening having a diameter of 20 mm) is greater than the force required to compress a gastric residence system having stiff arms to a bending configuration small enough to pass through a pylorus (i.e., an opening having a diameter of 20 mm). Thus, the radial force test results suggest that a gastric residence system having flexible arms is more able to resist premature passage through a patient's pylorus.

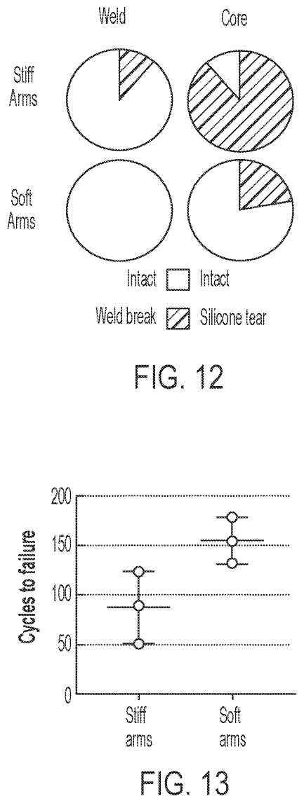

Example 3: FIG. 12 shows results of a double funnel durability test of two different gastric residence systems. In particular, FIG. 12 shows gastric residence system condition after 200 cycles in the double funnel test (described in detail above). This test was not performed in an aqueous environment.

As shown in the figure, a gastric residence system with stiff arms and a gastric residence system with flexible arms were tested. The gastric residence system with flexible arms comprised formula IA36 (see Table 1, above). The gastric residence system with stiff arms comprised formula IA37 (see Table 1, above). As shown, gastric residence systems with flexible arms resisted a connection failure (i.e., weld break) entirely, and greater than 75% resisted a silicone core tear failure. However, gastric residence systems with stiff arms showed some connection failures (i.e., weld break) (less than 12%) and greater than 85% silicone core tear failure. Accordingly, this test demonstrates that gastric residence systems comprising flexible arms may more effectively resist breaking and/or weakening (and thus, early pylorus passage) due to repeated gastric waves/forces in the stomach.

FIG. 13 shows the results of a double funnel test quantifying the number of cycles to failure. The gastric residence systems tested comprised of stiff arm materials (90 wt. % PCL, 10 wt. % sucrose) or flexible arm materials (29 wt. % PCL, 71 wt. % soluble materials, with soluble materials removed by solvent extraction prior to testing) joined to a silicone core with a disintegrating matrix. (Specifically, the 71 wt. % soluble materials comprised IVM119 (40% IVM, 20% Soluplus, 5% P407, 5% SSG, 0.5% Silica, 0.5% a-tocopherol, Balance PCL), incubated in ethanol prior to stellate assembly. Ethanol removes the ivermectin and excipients quickly, leaving a soft porous PCL arm.)

The disintegrating matrix comprised 15 wt. % polycaprolactone and 85 wt. % HPMCAS (hydroxypropyl methylcellulose acetate succinate). Three gastric residence systems of each formulation were subjected to repeated upwards and downwards motion in a double funnel test until two arms broke. The number of cycles was recorded. In each case of failure, breakage occurred at the joint between the disintegrating matrix and a neighboring material (i.e., core or arm). As shown in the Figure, gastric residence systems comprising flexible arms withstood more cycles prior to failure than gastric residence systems with stiff arms. This demonstrates that gastric residence systems having flexible arms may withstand more gastric compression waves before failure than gastric residence systems with stiff arms. Thus, gastric residence systems having flexible arms may be more effective at resisting premature failure and passage through a patient's pylorus.

Example 4: FIG. 14 shows the release of the water-soluble API dapagliflozin from elastic TPU-based matrices (i.e., materials that may be used for flexible arms). Drug release rate can be modulated by varying the content of soluble excipient (Kollidon VA64) in the formulation. Higher excipient content facilitates greater water entry into the matrix and faster drug release. Similarly, varying drug load within the matrix is expected to impact release rate, with higher drug loading creating greater porosity for accelerated release.

Dapagliflozin, TPU, and soluble excipients were combined in a hot melt extrusion process. Extrudates were shaped into triangular rods (representing gastric residence system arms, comprising an equilateral triangular cross-section having sides measuring 3.3 mm and a rod length measuring 15-20 mm) by compression molding or profile extrusion. Drug release from the matrix was measured by incubating the formulation in fasted state simulated gastric fluid (FaSSGF powder from BioRelevant) and measuring drug concentration in the release media over time. Release media was replaced at each sampling time point in order to maintain sink conditions based on drug solubility. Drug concentration in solution was measured by high performance liquid chromatography. Drug release is plotted as percent of loaded drug in the formulation. The specific formulation composition tested is as follows: 20% Amorphous dapagliflozin, 20% Bismuth subcarbonate, 0.5% silica, 0.5% vitamin E succinate, 0.1% iron oxide, Kollidon VA64, balance Pathways 72AE TPU.

Example 5: FIG. 15 shows dapagliflozin release from TPU-based matrices with and without a release-rate modulating polymer film. Drug polymer matrices were prepared by hot melt extrusion as described above with reference to FIG. 13. Extruded rods were cut into segments and coated with a polycaprolactone-based film using a pan coating process. In particular, coating components were dissolved in ethyl acetate and pan coating was performed using a Freund-Vector LDCS Hi-Coater Lab Coater.

As shown in the Figure, addition of the coating reduced initial burst release from the formulation and improved linearity of release overall. In particular, a greater coating weight provides a thicker diffusion barrier for slower release (see the 6% coating results as compared to the 3% coating results). It is expected that release rate may be further tuned by varying coating porosity, which may be achieved by changing the content of soluble excipient (Kollidon VA64) in the coating, with higher porosity leading to faster release.

As the underlying TPU matrices are flexible, it is feasible that bending of the matrix could cause coating disruption and loss of control over drug release. To evaluate this, a set of coated matrices were bent to a greater than 90-degree bend prior to the drug release assay (3% coat, bent prior to release and 6% coat, bent prior to release data points). Release data show that this bending had little to no impact on subsequent drug release, suggesting that the coating remained an intact diffusion barrier.

The formulation compositions used in this example are as follows:

Matrix (DaEX18): 20% Dapagliflozin Amorphous, 20% Bismuth subcarbonate, 0.5% silica, 0.5% vitamin E succinate, 0.1% iron oxide, 20% Kollidon VA64, balance Pathways 72AE TPU.

Coating: 73.5% PCL, 25% Kollidon VA64, 1.5% magnesium stearate

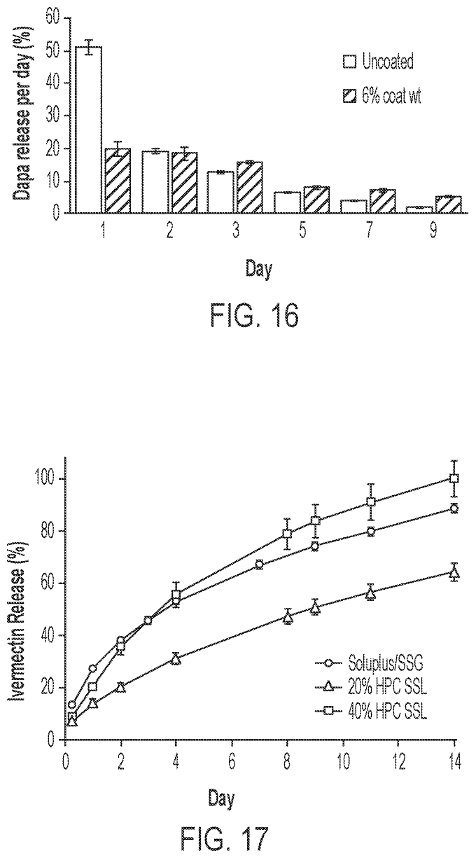

Example 6: FIG. 16 shows an alternate analysis of drug release from two of the formulations described above with respect to FIG. 14 (i.e., uncoated gastric residence system arms and 6% coat weight gastric residence arms). Specifically, the daily release of drug for the uncoated matrix and the matrix with a 6% coat weight of the PCL/copovidone blend was tested. For the uncoated matrix, the amount of drug delivered on Day 7 was less than 10% of the amount of drug delivered on Day 1. However, the amount of drug release for the coated arms was approximately 25% on Day 7 as compared to the amount of drug release on Day 1. Thus, the amount of drug release each day for the coated arms is more consistent and stable than that of the uncoated arms. This demonstrates that the addition of the coating on the loaded arms of a gastric residence system limits burst release on Day 1 and also helps to maintain sustained release at later time points.

Example 7: FIG. 17 shows release of the hydrophobic drug ivermectin from elastic TPU-based matrices. These matrices were prepared using the same methods as described above with reference to FIG. 14. The drug release rate can be modulated by varying the content and type of soluble excipients (in this case, Soluplus, sodium starch glycolate (SSG), and hydroxypropyl cellulose (HPC)) in the formulation. Higher excipient content can facilitate greater water entry into the matrix and faster drug release. Similarly, the amount of drug loading within the matrix is expected to impact release rate. Specifically, a higher drug loading can create greater porosity and an accelerated release.

The formulation compositions used to obtain the data provided in FIG. 16 include:

Soluplus/sodium starch glycolate (SSG): 20% ivermectin 40% Soluplus, 5% SSG, 5% P407, 0.5% Silica, 0.5% a-tocopherol succinate, Balance 72ATPU

20% hydroxypropylcellulose (HPC) SSL: 20% ivermectin, 20% HPC SSL, 5% P407, 0.5% Silica, 0.5% a-tocopherol succinate, Balance 72ATPU

40% HPC SSL: 20% ivermectin, 40% HPC SSL, 5% P407, 0.5% Silica, 0.5% a-tocopherol succinate, Balance 72ATPU

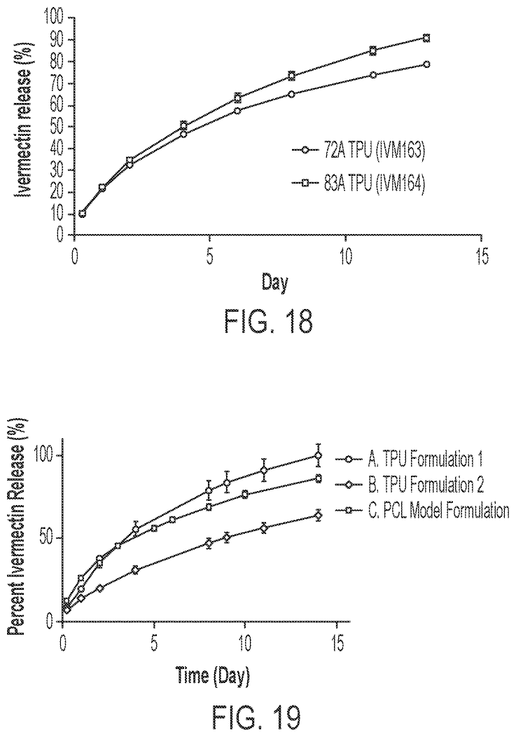

Example 8: FIG. 18 shows release of ivermectin from similar formulations (of those describe immediately above with reference to FIG. 16) prepared using different durometers of Pathways TPU. As shown in the figure, the release rate is similar for the two formulations. This suggests that TPU in varying durometers has a similar impact on controlling water entry and drug release. By varying TPU durometer, the overall stiffness of the gastric residence system arm may be modulated to meet targets for gastric retention. The data also suggest that durometer changes may be made with minimal impact to drug release profiles.

The formulation compositions used to obtain the results of depicted in FIG. 17 include 40% ivermectin, 20% Soluplus, 5% P407, 5% SSG, 0.5% Silica, 0.5% a-tocopherol, Balance TPU (83A or 72A durometer, Pathway PY-PT72AE or PY-PT83AL).

Example 9: FIG. 19 shows the release of the hydrophobic drug ivermectin from TPU and PCL matrices. To prepare the samples, a mixture of IVM, hydrophilic excipients, and base polymer TPU PYPT72AE were melt extruded via a lab scale Thermofisher Haake MiniCTW extruder. The ivermectin content was held at 20%, excipient content ranged from 25% to 45%, and TPU base polymer was remainder percent component. Extruded formulations were analyzed for drug release over 14 days by incubation in 0.01 M phosphate buffer (pH=7.0 with 0.5% of sodium dodecyl sulfate).

As shown in the Figure, PYPT72AE TPU with 45% excipient had higher extent of release then model PCL formulation. Moreover, ivermectin extent of release and excipient percentage have positive correlation.

Example 10: To evaluate shape retention of PCL and TPU at elevated temperatures, extruded rods (triangular cross section, 3.3 mm/side) were placed two supports across an unsupported 10-cm span in a controlled temperature oven. The center point of the rod was marked and its height was measured as the rods were exposed to increasing temperature (Table 1). PCL rods melted completely upon exposure to 60° C. and TPU rods maintained their shape at 75° C. At temperatures of 85-105° C., TPU appeared to soften slightly as the center point had lowered by ˜0.3 cm. TPU softened more rapidly at temperatures >105° C. The data suggest that TPU based gastric residence systems may have superior temperature stability when compared to PCL-based systems. The table below shows the results of this test.

| Temperature, | | |
|---|---|---|
| Exposure Time | PCL | TPU |
| RT- 50° C., days | Solid | Solid |
| 60° C., 30 min | Melted | No Change |
| 65° C., 1 h | Melted | No Change |
| 75° C., 1 h | Melted | No Change |
| 85° C., 1 h | Melted | 0.3 cm Sagging |
| 95° C., 1 h | Melted | 0.3 cm Sagging |
| 105° C., 1 h | Melted | 0.3 cm Sagging |
| 115° C., 1 h | Melted | 1.2 cm Sagging |
| 125° C., 1 h | Melted | 2.2 cm Sagging |
| 125° C., overnight | Melted | Melted |

Physical stability of polycaprolactone and Pathway PY-PT72AE TPU at elevated temperatures

Shape retention of encapsulated PCL- and TPU-based gastric residence systems was evaluated at excursion temperatures. Placebo arms made of PCL or TPU were assembled with silicone-based elastomeric cores to create stellate gastric residence systems. The stellates were folded and stored in 00EL HPMC capsules in an oven at 65° C. for 8h, then cooled and removed from capsules. The PCL arms melted and adhered to one another and the stellate was not able to open. The TPU (Pathway PY-PT72AE) arms remained separated triangular rods and the stellate unfolded intact.

### Exemplary Embodiments

Embodiment 1. A gastric residence system comprising: one or more arms extending radially, wherein the one or more arms comprises a first segment comprising a first polymer composition and a second segment comprising a second polymer composition, wherein the first segment has a stiffness that is greater than a stiffness of the second segment, as measured using a 3-point bending test per ASTM D790.

Embodiment 2. The gastric residence system of embodiment 1, comprising a core.

Embodiment 3. The gastric residence system of embodiment 2, comprising a plurality of arms connected to the core and extending radially from the core.

Embodiment 4. The gastric residence system of embodiment 3, wherein each arm of the plurality of arms comprises a first segment comprising a first polymer composition and a second segment comprising a second polymer composition.

Embodiment 5. The gastric residence system of any of embodiments 1-4, wherein the force required to compress the gastric residence system into a configuration small enough to pass through an opening having a diameter of 20 mm is at least 1.2 times greater than the force required to compress a gastric residence system having only a first polymer composition into a configuration small enough to pass through the opening, as measured using an iris testing mechanism.

Embodiment 6. The gastric residence system of any of embodiments 1-5, wherein the force required to compress the gastric residence system into a configuration small enough to pass through an opening having a diameter of 20 mm is 5-10 N, as measured using an iris testing mechanism.

Embodiment 7. The gastric residence system of any of embodiments 1-6, wherein the first polymer composition comprises one or more of PCL, PLA, PLGA, HPMCAS, and TPU.

Embodiment 8. The gastric residence system of any of embodiments 1-7, wherein the second polymer composition comprises one or more of a polyurethane, a polyether-polyamide copolymer, a thermoplastic elastomer, a thermoplastic polyurethane, polycaprolactone polylactic acid copolymer, a poly(trimethylene carbonate), a polyglycerol sebacate, and a silicone.

Embodiment 9. The gastric residence system of any of embodiments 1-8, wherein the second polymer composition comprises at least a polycaprolactone and a soluble material to form a material that softens upon exposure to an aqueous environment.

Embodiment 10. The gastric residence system of any of embodiments 1-9, wherein the first segment is directly connected to the second segment of the at least one arm.

Embodiment 11. The gastric residence system of any of embodiments 1-9, wherein the first segment is connected to the second segment via a linker.

Embodiment 12. The gastric residence system of any of embodiments 1-11, wherein the first segment comprises 20-50% of a length of the at least one arm, the length being measured from a proximal end of the at least one arm, the proximal end being proximate to the core or a linker connecting the at least one arm to the core, to a distal end of the at least one arm.

Embodiment 13. The gastric residence system of any embodiments 1-12, wherein the second segment comprises 50-80% of a length of the at least one arm, the length being measured from a proximal end of the at least one arm, the proximal end being proximate to the core or a linker connecting the at least one arm to the core, to a distal end of the at least one arm.

Embodiment 14. The gastric residence system of any of embodiments 1-13, wherein a number of fatigue cycles required to break the gastric residence system is at least 25% greater than a number of fatigue cycles required to break a gastric residence system with stiff arms, as measured using a double funnel test.

Embodiment 15. The gastric residence system of any of embodiments 1-14, comprising a filament circumferentially connecting the distal end of each arm of the plurality of arms.

Embodiment 16. The gastric residence system of embodiment 15, wherein the force required to compress the gastric residence system into a configuration small enough to pass through an opening having a diameter of 20 mm is at least one and a half times greater than the force required to compress a gastric residence system without a filament into a configuration small enough to pass through the opening, as measured using an iris testing mechanism.

Embodiment 17. The gastric residence system of embodiment 15 or 16, wherein the distal end of each arm of the plurality of arms comprises an enteric polymer composition.

Embodiment 18. The gastric residence system of any of embodiments 15-17, wherein the filament comprises one or more of an elastic polymer, a biosorbable polymer, and a plasticizer.

Embodiment 19. The gastric residence system of embodiment 17 or 18, wherein the enteric polymer composition comprises a biodegradable polymer, an enteric polymer, a plasticizer, and an acid.

Embodiment 20. The gastric residence system of embodiment 19, wherein the biodegradable polymer comprises polycaprolactone.

Embodiment 21. The gastric residence system of embodiment 19 or 20, wherein the enteric polymer comprises hydroxypropylmethylcellulose acetate succinate.

Embodiment 22. The gastric residence system of any of embodiments 19-21, wherein the plasticizer comprises propylene glycol.

Embodiment 23. The gastric residence system of any of embodiments 19-22, wherein the acid comprises stearic acid.

Embodiment 24. The gastric residence system of any of embodiments 15-23, wherein the pullout force required to separate the filament from the distal end of a first arm of the plurality of arms is greater than 1N when measured after incubating the gastric residence system in an environment of pH 1.6 for 3 days.

Embodiment 25. The gastric residence system of any of embodiments 15-24, wherein the pullout force required to separate the filament from the distal end of the first arm of the plurality of arms is less than 2N when measured after incubating the gastric residence system in an environment of pH 6.5 for 3 days.

Embodiment 26. The gastric residence system of any of embodiments 15-25, wherein the distal end of each arm comprises a notch and the filament is positioned within the notch of each distal end.

Embodiment 27. The gastric residence system of embodiment 26, wherein the filament is secured by overlapping a first end of the filament and a second end of the filament within a first notch, and the first end and the second end are secured by one of knotting or heat flaring.

Embodiment 28. The gastric residence system of any of embodiments 1-27, wherein the gastric residence system is used to treat a patient.

Embodiment 29. The gastric residence system of embodiment 28, wherein the patient is a human or a dog.

Embodiment 30. A method of manufacturing a gastric residence system comprising:

connecting a first material comprising a first polymer composition to a second material comprising a second polymer composition to form a first arm comprising a first segment at a first end of the arm and a second segment at a second end of the first arm;

connecting a third material comprising a third polymer composition to a fourth material comprising a fourth polymer composition to form a second arm comprising a third segment at a third end of the arm and a fourth portion at a fourth end of the second arm;

connecting the first end of the first arm and the third end of the second arm to a core to form a gastric residence system comprising a core, a first arm, and a second arm, the first arm and the second arm extending radially from the core, wherein the first segment of the first arm comprises a first proximal end and the third segment of the second arm comprises a second proximal end.

Embodiment 31. The method of embodiment 30, wherein the first material and the third material are the same and the first polymer composition and the third polymer composition are the same.

Embodiment 32. The method of embodiment 30 or 31, wherein the second material and the fourth material are the same and the second polymer composition and the fourth polymer composition are the same.

Embodiment 33. The method of any of embodiments 30-32, wherein the first segment and the third segment have a stiffness of greater a stiffness of the second segment and the fourth portion, as measured using a 3-point bending test per ASTM D790.

Embodiment 34. The method of any of embodiments 30-33, comprising more than two arms connected to the core and extending radially from the core.

Embodiment 35. The method of embodiment 34, wherein each arm of more than two arms comprises a first segment comprising a first polymer composition and a second segment comprising a second polymer composition or a third segment comprising a third polymer composition and a fourth portion comprising a fourth polymer composition.

Embodiment 36. The method of any of embodiments 30-35, wherein the force required to compress the gastric residence system into a configuration small enough to pass through an opening having a diameter of 20 mm is at least 1.2 times greater than the force required to compress a gastric residence system having arms comprising only a first polymer composition or a third polymer composition into a configuration small enough to pass through the opening, as measured using an iris testing mechanism.

Embodiment 37. The gastric residence system of any of embodiments 30-36, wherein the force required to compress the gastric residence system into a configuration small enough to pass through an opening having a diameter of 20 mm is 5-10 N, as measured using an iris testing mechanism.

Embodiment 38. The method of any of embodiments 30-37, wherein the first polymer composition and the third polymer composition comprise one or more of PCL, PLA, PLGA, HPMCAS, and TPU.

Embodiment 39. The method of any of embodiments 30-38, wherein the second polymer composition and the fourth polymer composition comprise one or more of a polyurethane, a polyether-polyamide copolymer, a thermoplastic elastomer, a thermoplastic polyurethane, polycaprolactone polylactic acid copolymer, a poly(trimethylene carbonate), a polyglycerol sebacate, and a silicone.

Embodiment 40. The method of any of embodiments 30-39, wherein the second polymer composition and the fourth polymer composition comprise at least a polycaprolactone and a soluble material to form a material that softens upon exposure to an aqueous environment.

Embodiment 41. The method of any of embodiments 30-40, wherein the first segment is directly connected to the second segment of the first arm and the third segment is directly connected to the fourth portion of the second arm.

Embodiment 42. The method of any of embodiments 30-41, wherein the first segment is connected to the second segment via a linker and the third segment is connected to the fourth portion via a linker.

Embodiment 43. The method of any of embodiments 30-42, wherein the first segment comprises 20-50% of a length of the first arm, the length being measured from a proximal end of the first arm, the proximal end being proximate to the core, to a distal end of the first arm.

Embodiment 44. The method of any of embodiments 30-43, wherein the third segment comprises 20-50% of a length of the second arm, the length being measured from a proximal end of the second arm, the proximal end being proximate to the core, to a distal end of the second arm.

Embodiment 45. The method of any of embodiments 30-44, wherein the second segment comprises 50-80% of a length of the first arm, the length being measured from a proximal end of the first arm, the proximal end being proximate to the core, to a distal end of the first arm.

Embodiment 46. The method of any of embodiments 30-45, wherein the fourth portion comprises 50-80% of a length of the second arm, the length being measured from a proximal end of the second arm, the proximal end being proximate to the core, to a distal end of the second arm.

Embodiment 47. The method of any of embodiments 30-46, wherein a number of fatigue cycles required to break the gastric residence system is at least 25% greater than a number of fatigue cycles required to break a gastric residence system with arms comprising only a first segment or a third segment, as measured using a double funnel test.

Embodiment 48. The method of any of embodiments 30-47, comprising wrapping a filament circumferentially connecting the distal end of the first arm and the second arm.

Embodiment 49. The method of embodiment 48, comprising wrapping a filament circumferentially connecting the distal end of each of the more than two arms.

Embodiment 50. The method of embodiment 48 or 49, wherein the force required to compress the gastric residence system into a configuration small enough to pass through an opening having a diameter of 20 mm is at least 1.2 times greater than the force required to compress a gastric residence system comprising arms having only a first polymer composition or a third polymer composition and without a filament into a configuration small enough to pass through the opening, as measured using an iris testing mechanism.

Embodiment 51. The method of embodiment 48, wherein the distal end of each arm of the first arm and the second arm comprises an enteric polymer composition.

Embodiment 52. The method of embodiment 49, wherein the distal end of each arm of the two or more arms comprises an enteric polymer composition.

Embodiment 53. The method of any of embodiments 48-52, wherein the filament comprises one or more of an elastic polymer, a biosorbable polymer, and a plasticizer.

Embodiment 54. The method of any of embodiments 51-53, wherein the enteric polymer composition comprises a biodegradable polymer, an enteric polymer, a plasticizer, and an acid.

Embodiment 55. The method of embodiment 54, wherein the biodegradable polymer comprises polycaprolactone.

Embodiment 56. The method of embodiment 54 or 55, wherein the enteric polymer comprises hydroxypropylmethylcellulose acetate succinate.

Embodiment 57. The method of any of embodiments 54-56, wherein the plasticizer comprises propylene glycol.

Embodiment 58. The method of any of embodiments 54-57, wherein the acid comprises stearic acid.

Embodiment 59. The method of any of embodiments 48-58, wherein the pullout force required to separate the filament from the distal end of the first arm or the second arm is greater than 1N when measured after incubating the gastric residence system in an environment of pH 1.6 for 3 days.

Embodiment 60. The method of any of embodiments 48-59, wherein the pullout force required to separate the filament from the distal end of the first arm or the second arm is less than 2N when measured after incubating the gastric residence system in an environment of pH 6.5 for 3 days.

Embodiment 61. The method of any of embodiments 48-60, wherein the distal end of the first arm and the distal end of the second arm comprises a notch, and the filament is positioned within the notch of each distal end.

Embodiment 62. The method of embodiment 61, wherein the filament is secured by overlapping a first end of the filament and a second end of the filament within a first notch, and the first end and the second end are secured by one of knotting or heat flaring.

Embodiment 63. A gastric residence system made using the method of any of embodiments 30-62, wherein the gastric residence system is used to treat a patient.

Embodiment 64. The gastric residence system of embodiment 63, wherein the patient is a human or a dog.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference to “about” a value or parameter herein includes (and describes) variations that are directed to that value or parameter per se. For example, description referring to “about X” includes description of “X”.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

1. A gastric residence system comprising:
one or more arms extending radially, wherein the one or more arms comprises a first segment comprising a first polymer composition and a second segment comprising a second polymer composition, wherein the first segment has a stiffness that is greater than a stiffness of the second segment, as measured using a 3-point bending test per ASTM D790.

2. The gastric residence system of claim 1, comprising a core.

3. The gastric residence system of claim 2, comprising a plurality of arms connected to the core and extending radially from the core.

4. The gastric residence system of claim 3, wherein each arm of the plurality of arms comprises a first segment comprising a first polymer composition and a second segment comprising a second polymer composition.

5. The gastric residence system of any of claims 1-4, wherein the force required to compress the gastric residence system into a configuration small enough to pass through an opening having a diameter of 20 mm is at least 1.2 times greater than the force required to compress a gastric residence system having only a first polymer composition into a configuration small enough to pass through the opening, as measured using an iris testing mechanism.

6. The gastric residence system of any of claims 1-5, wherein the force required to compress the gastric residence system into a configuration small enough to pass through an opening having a diameter of 20 mm is 5-10 N, as measured using an iris testing mechanism.

7. The gastric residence system of any of claims 1-6, wherein the first polymer composition comprises one or more of PCL, PLA, PLGA, HPMCAS, and TPU.

8. The gastric residence system of any of claims 1-7, wherein the second polymer composition comprises one or more of a polyurethane, a polyether-polyamide copolymer, a thermoplastic elastomer, a thermoplastic polyurethane, polycaprolactone polylactic acid copolymer, a poly(trimethylene carbonate), a polyglycerol sebacate, and a silicone.

9. The gastric residence system of any of claims 1-8, wherein the second polymer composition comprises at least a polycaprolactone and a soluble material to form a material that softens upon exposure to an aqueous environment.

10. The gastric residence system of any of claims 1-9, wherein the first segment is directly connected to the second segment of the at least one arm.

11. The gastric residence system of any of claims 1-9, wherein the first segment is connected to the second segment via a linker.

12. The gastric residence system of any of claims 1-11, wherein the first segment comprises 20-50% of a length of the at least one arm, the length being measured from a proximal end of the at least one arm, the proximal end being proximate to the core or a linker connecting the at least one arm to the core, to a distal end of the at least one arm.

13. The gastric residence system of any claims 1-12, wherein the second segment comprises 50-80% of a length of the at least one arm, the length being measured from a proximal end of the at least one arm, the proximal end being proximate to the core or a linker connecting the at least one arm to the core, to a distal end of the at least one arm.

14. The gastric residence system of any of claims 1-13, wherein a number of fatigue cycles required to break the gastric residence system is at least 25% greater than a number of fatigue cycles required to break a gastric residence system with stiff arms, as measured using a double funnel test.

15. The gastric residence system of any of claims 1-14, comprising a filament circumferentially connecting the distal end of each arm of the plurality of arms.

16. The gastric residence system of claim 15, wherein the force required to compress the gastric residence system into a configuration small enough to pass through an opening having a diameter of 20 mm is at least one and a half times greater than the force required to compress a gastric residence system without a filament into a configuration small enough to pass through the opening, as measured using an iris testing mechanism.

17. The gastric residence system of claim 15 or 16, wherein the distal end of each arm of the plurality of arms comprises an enteric polymer composition.

18. The gastric residence system of any of claims 15-17, wherein the filament comprises one or more of an elastic polymer, a biosorbable polymer, and a plasticizer.

19. The gastric residence system of claim 17 or 18, wherein the enteric polymer composition comprises a biodegradable polymer, an enteric polymer, a plasticizer, and an acid.

20. The gastric residence system of claim 19, wherein the biodegradable polymer comprises polycaprolactone.

21. The gastric residence system of claim 19 or 20, wherein the enteric polymer comprises hydroxypropylmethylcellulose acetate succinate.

22. The gastric residence system of any of claims 19-21, wherein the plasticizer comprises propylene glycol.

23. The gastric residence system of any of claims 19-22, wherein the acid comprises stearic acid.

24. The gastric residence system of any of claims 15-23, wherein the pullout force required to separate the filament from the distal end of a first arm of the plurality of arms is greater than 1N when measured after incubating the gastric residence system in an environment of pH 1.6 for 3 days.

25. The gastric residence system of any of claims 15-24, wherein the pullout force required to separate the filament from the distal end of the first arm of the plurality of arms is less than 2N when measured after incubating the gastric residence system in an environment of pH 6.5 for 3 days.

26. The gastric residence system of any of claims 15-25, wherein the distal end of each arm comprises a notch and the filament is positioned within the notch of each distal end.

27. The gastric residence system of claim 26, wherein the filament is secured by overlapping a first end of the filament and a second end of the filament within a first notch, and the first end and the second end are secured by one of knotting or heat flaring.

28. The gastric residence system of any of claims 1-27, wherein the gastric residence system is used to treat a patient.

29. The gastric residence system of claim 28, wherein the patient is a human or a dog.

30. A method of manufacturing a gastric residence system comprising:
connecting a first material comprising a first polymer composition to a second material comprising a second polymer composition to form a first arm comprising a first segment at a first end of the arm and a second segment at a second end of the first arm;
connecting a third material comprising a third polymer composition to a fourth material comprising a fourth polymer composition to form a second arm comprising a third segment at a third end of the arm and a fourth portion at a fourth end of the second arm;
connecting the first end of the first arm and the third end of the second arm to a core to form a gastric residence system comprising a core, a first arm, and a second arm, the first arm and the second arm extending radially from the core, wherein the first segment of the first arm comprises a first proximal end and the third segment of the second arm comprises a second proximal end.

31. The method of claim 30, wherein the first material and the third material are the same and the first polymer composition and the third polymer composition are the same.

32. The method of claim 30 or 31, wherein the second material and the fourth material are the same and the second polymer composition and the fourth polymer composition are the same.

33. The method of any of claims 30-32, wherein the first segment and the third segment have a stiffness of greater a stiffness of the second segment and the fourth portion, as measured using a 3-point bending test per ASTM D790.

34. The method of any of claims 30-33, comprising more than two arms connected to the core and extending radially from the core.

35. The method of claim 34, wherein each arm of more than two arms comprises a first segment comprising a first polymer composition and a second segment comprising a second polymer composition or a third segment comprising a third polymer composition and a fourth portion comprising a fourth polymer composition.

36. The method of any of claims 30-35, wherein the force required to compress the gastric residence system into a configuration small enough to pass through an opening having a diameter of 20 mm is at least 1.2 times greater than the force required to compress a gastric residence system having arms comprising only a first polymer composition or a third polymer composition into a configuration small enough to pass through the opening, as measured using an iris testing mechanism.

37. The gastric residence system of any of claims 30-36, wherein the force required to compress the gastric residence system into a configuration small enough to pass through an opening having a diameter of 20 mm is 5-10 N, as measured using an iris testing mechanism.

38. The method of any of claims 30-37, wherein the first polymer composition and the third polymer composition comprise one or more of PCL, PLA, PLGA, HPMCAS, and TPU.

39. The method of any of claims 30-38, wherein the second polymer composition and the fourth polymer composition comprise one or more of a polyurethane, a polyether-polyamide copolymer, a thermoplastic elastomer, a thermoplastic polyurethane, polycaprolactone polylactic acid copolymer, a poly(trimethylene carbonate), a polyglycerol sebacate, and a silicone.

40. The method of any of claims 30-39, wherein the second polymer composition and the fourth polymer composition comprise at least a polycaprolactone and a soluble material to form a material that softens upon exposure to an aqueous environment.

41. The method of any of claims 30-40, wherein the first segment is directly connected to the second segment of the first arm and the third segment is directly connected to the fourth portion of the second arm.

42. The method of any of claims 30-41, wherein the first segment is connected to the second segment via a linker and the third segment is connected to the fourth portion via a linker.

43. The method of any of claims 30-42, wherein the first segment comprises 20-50% of a length of the first arm, the length being measured from a proximal end of the first arm, the proximal end being proximate to the core, to a distal end of the first arm.

44. The method of any of claims 30-43, wherein the third segment comprises 20-50% of a length of the second arm, the length being measured from a proximal end of the second arm, the proximal end being proximate to the core, to a distal end of the second arm.

45. The method of any of claims 30-44, wherein the second segment comprises 50-80% of a length of the first arm, the length being measured from a proximal end of the first arm, the proximal end being proximate to the core, to a distal end of the first arm.

46. The method of any of claims 30-45, wherein the fourth portion comprises 50-80% of a length of the second arm, the length being measured from a proximal end of the second arm, the proximal end being proximate to the core, to a distal end of the second arm.

47. The method of any of claims 30-46, wherein a number of fatigue cycles required to break the gastric residence system is at least 25% greater than a number of fatigue cycles required to break a gastric residence system with arms comprising only a first segment or a third segment, as measured using a double funnel test.

48. The method of any of claims 30-47, comprising wrapping a filament circumferentially connecting the distal end of the first arm and the second arm.

49. The method of claim 48, comprising wrapping a filament circumferentially connecting the distal end of each of the more than two arms.

50. The method of claim 48 or 49, wherein the force required to compress the gastric residence system into a configuration small enough to pass through an opening having a diameter of 20 mm is at least 1.2 times greater than the force required to compress a gastric residence system comprising arms having only a first polymer composition or a third polymer composition and without a filament into a configuration small enough to pass through the opening, as measured using an iris testing mechanism.

51. The method of claim 48, wherein the distal end of each arm of the first arm and the second arm comprises an enteric polymer composition.

52. The method of claim 49, wherein the distal end of each arm of the two or more arms comprises an enteric polymer composition.

53. The method of any of claims 48-52, wherein the filament comprises one or more of an elastic polymer, a biosorbable polymer, and a plasticizer.

54. The method of any of claims 51-53, wherein the enteric polymer composition comprises a biodegradable polymer, an enteric polymer, a plasticizer, and an acid.

55. The method of claim 54, wherein the biodegradable polymer comprises polycaprolactone.

56. The method of claim 54 or 55, wherein the enteric polymer comprises hydroxypropylmethylcellulose acetate succinate.

57. The method of any of claims 54-56, wherein the plasticizer comprises propylene glycol.

58. The method of any of claims 54-57, wherein the acid comprises stearic acid.

59. The method of any of claims 48-58, wherein the pullout force required to separate the filament from the distal end of the first arm or the second arm is greater than 1N when measured after incubating the gastric residence system in an environment of pH 1.6 for 3 days.

60. The method of any of claims 48-59, wherein the pullout force required to separate the filament from the distal end of the first arm or the second arm is less than 2N when measured after incubating the gastric residence system in an environment of pH 6.5 for 3 days.

61. The method of any of claims 48-60, wherein the distal end of the first arm and the distal end of the second arm comprises a notch, and the filament is positioned within the notch of each distal end.

62. The method of claim 61, wherein the filament is secured by overlapping a first end of the filament and a second end of the filament within a first notch, and the first end and the second end are secured by one of knotting or heat flaring.

63. A gastric residence system made using the method of any of claims 30-62, wherein the gastric residence system is used to treat a patient.

64. The gastric residence system of claim 63, wherein the patient is a human or a dog.
